SEQUENCE LISTING (39 sequences)

SEQ ID NO: 1 - protein, 141 aa, Vicugna pacos
QVQLQESGGGLVQAGGSLRLSCAASGRFFSSYAMGWFRQAPGKEREFVAAISWSGGSTYYADSVKGRFTTSRDNAKNTVYLLMNSLKPEDTAVYYCAAAREGAYYPDSYYRTVRYDYWGQGTQVTVSSAAAYPYDVPDYGS

SEQ ID NO: 2 - protein, 130 aa, Vicugna pacos
QVQLQESGGGLVQAGGSLRLSCAASGRTFSSYRMGWYRQAPGKEREFVAAITGSGDSTNYADSVKGRFTVSGNSARNLVYLQMNSLKPEDTAVYLCVAYRTGGPPQWGQGTQVTVSSAAAYPYDVPDYGS

SEQ ID NO: 3 - protein, 137 aa, Vicugna pacos
QVQLQESGGGLVQAGGSLRLSCTVSGRTDSESTMGWFRQAAGKGREFVAAMNWRYATTYHTDSVKGRFTISKDSAKNTMYLQMNSLKPEDTAVYYCAHRYIYGSLSDSGSYDNWGQGTQVTVSSAAAYPYDVPDYGS

SEQ ID NO: 4 - protein, 119 aa, Vicugna pacos
QVQLQESGGGLVQAGGSLRLSCAASGTIFSIDAFGWYRQAPGKQREWVAGITSGSSTIYADFVKGRFTISRDNAKNTVFLQMNSLKPEDTAVYYCNRAKPPTYYSLEPWGKGTQVTVSS

SEQ ID NO: 5 - protein, 10 aa, Vicugna pacos
ASGRFFSSYA

SEQ ID NO: 6 - protein, 8 aa, Vicugna pacos
ISWSGGST

SEQ ID NO: 7 - protein, 18 aa, Vicugna pacos
AREGAYYPDSYYRTVRYD

SEQ ID NO: 8 - protein, 9 aa, Vicugna pacos
ASGRTFSSY

SEQ ID NO: 9 - protein, 5 aa, Vicugna pacos
TGSGD

SEQ ID NO: 10 - protein, 9 aa, Vicugna pacos
YRTGGPPQW

SEQ ID NO: 11 - protein, 7 aa, Vicugna pacos
RTDSEST

SEQ ID NO: 12 - protein, 4 aa, Vicugna pacos
WRYA

SEQ ID NO: 13 - protein, 15 aa, Vicugna pacos
RYIYGSLSDSGSYDN

SEQ ID NO: 14 - protein, 9 aa, Vicugna pacos
SGTIFSIDA

SEQ ID NO: 15 - protein, 8 aa, Vicugna pacos
QAPGKQRE

SEQ ID NO: 16 - protein, 16 aa, Vicugna pacos
AKPPTYYSLEPWGKGT

SEQ ID NO: 17 - DNA, 444 bp, Vicugna pacos
  source 1..444: mol_type = genomic DNA
caggtgcagctgcaggagtctgggggaggattggtgcaggctgggggctctctgagactctcctgtgcagcctctggacgcttcttcagtagttatgccatgggctggttccgccaggctccaggaaaagagcgtgagtttgtagcagccattagctggagtggtggtagcacatactatgcagactccgtgaagggccgattcaccacctccagagacaacgccaagaacacggtgtatctgctaatgaacagcctgaaacctgaggacacggccgtttattactgtgcagcagcgcgggagggggcatactatcctgatagttactaccgcaccgtgaggtatgactactggggccaggggacccaggtcaccgtctcctcagcggccgcatacccgtacgacgttccggactacggttcccaccaccatcaccatcactag

SEQ ID NO: 18 - DNA, 411 bp, Vicugna pacos
  source 1..411: mol_type = genomic DNA
caggtgcagctgcaggagtctgggggaggattggtgcaggctgggggctctctgagactctcctgtgcagcctctggacgcacgtttagtagttataggatgggctggtatcgccaggctccagggaaggagcgtgagtttgtagcagctattacggggagcggcgatagcacaaactatgcagactccgtgaagggccgattcaccgtctccggaaacagcgccaggaacttggtgtatttgcaaatgaacagcctgaaacctgaggacacggccgtttatctctgtgtagcttaccgtactggtgggcccccgcaatggggccaggggacccaggtcaccgtctcctcagcggccgcatacccgtacgacgttccggactacggttcccaccaccatcaccatcactag

SEQ ID NO: 19 - DNA, 432 bp, Vicugna pacos
  source 1..432: mol_type = genomic DNA
caggtgcagctgcaggagtctgggggaggattggtgcaggctgggggctctctgagactctcctgtacagtctctggacgcactgacagtgaaagtacgatgggctggttccgccaggctgcagggaaggggcgtgagtttgtagcagctatgaactggagatacgcgaccacatatcatacagactccgtgaagggccgattcactatctccaaagacagcgccaagaacacaatgtatctgcaaatgaacagcctgaaacctgaggacacggccgtttattactgtgcgcaccgttatatatatggtagtctttccgattcaggatcgtatgacaactggggccaggggacccaggtcaccgtctcctcagcggccgcatacccgtacgacgttccggactacggttcccaccaccatcaccatcactag

SEQ ID NO: 20 - DNA, 417 bp, Vicugna pacos
  source 1..417: mol_type = genomic DNA
caggtgcagctgcaggagtctgggggaggcttggtgcaggctggggggtctctgagactctcctgtgcagcctctggaaccatcttcagtatcgatgccttcggctggtaccgccaggctccggggaagcagcgcgagtgggtcgctggtattactagtggtagtagtacaatatatgcagacttcgtgaagggccgattcaccatctccagagacaatgccaagaacacggtgtttctccaaatgaacagcctgaaacctgaggacacggccgtctattactgtaatagggccaagccccctacttactacagcctggagccctggggcaaagggacccaggtcaccgtctcctcagcggccgcatacccgtacgacgttccggactacggttcccaccaccatcaccatcactag

SEQ ID NO: 21 - protein, 540 aa, Norwalk virus
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 22 - protein, 548 aa, Norwalk virus
MKMASNDAAPSNDGAAGLVPESNNEVMALEPVAGASLAAPVTGQTNIIDPWIRMNFVQAPNGEFTVSPRNSPGEVLLNLELGPELNPYLAHLSRMYNGYAGGMEVQIMLAGNAFTAGKLIFAAVPPHFPIENLSPPQITMFPHVIIDVRTLEPVLLPMPDIRNSFFHFIQRDEPKMRLVAMLYTPLRSNGSGDDVFTVSCRVLTRPTPDFDFTYLVPPTVESKSKPFTLPILTLGELTNSRFPLPIDVLYTNPNESAIVQCQNGRCTLDGELQGTTQLLPTGICAFRGKVTQQVQDEHRGTHWNMTVTNLNGTPFDPTEDVPAPLGTPDFSGQIYGVISQRNTNTVPGEGNLPANRAHEAVIATYSPKFTPKLGNIQFSTWETQDVSSGQPTKFTPVGLASVDANSHFDQWTLPSYSGALTLNMNLAPSVAPVFPGECLLFFRSFIPLKGGYGNPAIDCLMPQEWVQHLYQESAPSLSDVALVRYVNPETGRTLFEAKLHRNGFLTVARNSAGPVVAPTNGYFRFDSWVNQFYTLAPMGNGSGRRRMQ

SEQ ID NO: 23 - protein, 540 aa, Norwalk virus
MKMASNDAAPSNDGAAGLVPEGNNETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYSNQPNSRMRLVAMLYTPLRSNGSGDDVFTVSCRVLTRPTPDFEFTYLVPPSVESKTKPFSLPILTLSELTNSRFPVPIDSLFTAQNNVLQVQCQNGRCTLDGELQGTTQLLPTGICAFRGRVTAQINQRDRWHMQLQNLNGTTYDPTDDVPAPLGTPDFKGVVFGMVSQRNVGNDAPGSTRAQQAWVSTYSPQFVPKLGSVNLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 24 - protein, 6 aa, synthetic construct
HHHHHH

SEQ ID NO: 25 - protein, 7 aa, synthetic construct
WVNXFYX

SEQ ID NO: 26 - protein, 8 aa, Vicugna pacos
GSIFSIYA

SEQ ID NO: 27 - protein, 8 aa, Vicugna pacos
GSIFSIYL

SEQ ID NO: 28 - protein, 8 aa, Vicugna pacos
ISSGGGTN

SEQ ID NO: 29 - protein, 14 aa, Vicugna pacos
KREDYSAYAPPSGS

SEQ ID NO: 30 - protein, 14 aa, Vicugna pacos
KREDFSAYAPPSGS

SEQ ID NO: 31 - protein, 135 aa, Vicugna pacos
DVQLVESGGGLVQPGGSLRLSCAASGSIFSIYAMGWYRQAPGKQRELVASISSGGGTNYADSVKGRFTISGDNAKNTVYLQMNSLKPEDTAVYYCKREDYSAYAPPSGSRGRGTQVTVSSGRYPYDVPDYGSGRA

SEQ ID NO: 32 - skipped

SEQ ID NO: 33 - protein, 8 aa, synthetic construct
DXELXGXT

SEQ ID NO: 34 - skipped

SEQ ID NO: 35 - protein, 19 aa, synthetic construct
YQESXPXXXXXXXXXXXXP

SEQ ID NO: 36 - protein, 8 aa, Vicugna pacos
RIIFFMYD

SEQ ID NO: 37 - protein, 8 aa, Vicugna pacos
QINSDVST

SEQ ID NO: 38 - protein, 9 aa, Vicugna pacos
YCNVRRASA

SEQ ID NO: 39 - protein, 115 aa, Vicugna pacos
QVQLQESGGGLVQPGGSLRLSCTAPRIIFFMYDVGWYRQAPEKQRELVAQINSDVSTKYADSVKGRFTISRDNAKRTVYLQMNDLKPEDAAVYYCNVRRASADYWGQGTQVTVSS

(19) United States
(12) Patent Application Publication
(10) Pub. No.: US 2026/0265349 A1
(43) Pub. Date: Sep. 10, 2026

(54) NANOBODY FOR NOROVIRUS

(71) Applicant: GRIFFITH UNIVERSITY, Nathan, Queensland (AU)

(72) Inventor: Grant HANSMAN, Nathan, Queensland (AU)

(21) Appl. No.: 19/163,363

(22) PCT Filed: Mar. 11, 2024
(86) PCT No.: PCT/AU2024/050213

§ 371 Date: Sep. 8, 2025

(30) Foreign Application Priority Data
Mar. 9, 2023 (AU) 2023900643

(51) Int. Cl. C07K 16/10; A61K 47/68; A61P 31/14; G01N 33/569

(52) U.S. Cl. CPC C07K 16/10; A61K 47/6841; A61P 31/14; G01N 33/56983; C07K 2317/24; C07K 2317/31; C07K 2317/33; C07K 2317/35; C07K 2317/565; C07K 2317/569; C07K 2317/76; G01N 2333/08

(57) ABSTRACT

The present disclosure relates to the field of nanobodies, therapeutic agents, compositions and methods for the prevention, amelioration and treatment of noroviral infections.

### CROSS-REFERENCE TO RELATED APPLICATIONS

The present application claims priority from Australian Provisional Patent Application No. 2023900643 filed on 9 Mar. 2024, the contents of which are incorporated herein by reference in their entirety.

### TECHNICAL FIELD

The present disclosure relates to the field of nanobodies, therapeutic agents, compositions and methods for the prevention, amelioration and treatment of noroviral infections.

### BACKGROUND

Human norovirus was discovered over half a century ago (1), yet there are still no vaccines, antivirals, or treatments currently available. These viruses are highly contagious and a major problem in closed institutions such as schools, hospitals, and cruise ships. Reducing norovirus infections is challenging on multiple levels and includes the frequent emergence of antigenic variants, which complicates designing effective, broadly reactive capsid therapeutics.

Accordingly, there remains an unmet clinical need for effective drug therapies for the prevention and treatment of noroviral infections.

### SUMMARY

The present disclosure is based on the surprising finding of a number of camelid-derived nanobodies that specifically bind a top portion of a P domain of a norovirus. Additionally, their demonstrated ability to effectively bind and potently inhibit the ability of these viruses to bind host co-factors that are necessary for infection makes these nanobodies suitable therapeutic agents for the prevention or treatment of noroviral infections. Such nanobodies can also have diagnostic or prognostic applications in relation to noroviral infections.

In a first aspect, the present disclosure provides a single domain antibody that is directed against a P domain of a norovirus, wherein the single domain antibody binds to or interacts with one or more residues of a P2 subdomain thereof.

Suitably, the P2 subdomain comprises, consists of or consists essentially of residues 275 to 417 or residues 276 to 418 of a VP1 protein of the norovirus. In various examples, the single domain antibody binds to or interacts with one or more residues of the P domain selected from the group consisting of LYS-329, THR-344, SER-355, ALA-356, ASP-357, GLU-368, ASP-391, THR-394, ASN-398, GLN-401 and GLY-443 according to the amino acid numbering of a VP1 protein of a GII.4 genotype. In other examples, the single domain antibody binds to or interacts with one or more residues of the P domain selected from the group consisting of ASN-295, GLN-296, ARG-297, ARG-299, GLN-352, TRP-354, GLN-361, ARG-372, SER-374, ASN-392, ASP-393, ASP-394, ASP-395, ASP-396, SER-441, GLY-442, GLY-443 and TYR-444 according to the amino acid numbering of a VP1 protein of a GII.17 genotype.

In certain examples, the single domain antibody comprises:
(a) a CDR1 that comprises an amino acid sequence of ASGRFFSSYA (SEQ ID NO: 5), ASGRTFSSY (SEQ ID NO: 8), RTDSEST (SEQ ID NO: 11), SGTIFSIDA (SEQ ID NO: 14) or a variant thereof;
(b) a CDR2 that comprises an amino acid sequence of ISWSGGST (SEQ ID NO: 6), TGSGD (SEQ ID NO: 9), WRYA (SEQ ID NO: 12), QAPGKQRE (SEQ ID NO: 15) or a variant thereof; and
(c) a CDR3 that comprises an amino acid sequence of AREGAYYPDSYYRTVRYD (SEQ ID NO: 7), YRTGGPPQW (SEQ ID NO: 10), RYIYGSLSDSGSYDN (SEQ ID NO: 13), AKPPTYYSLEPWGKGT (SEQ ID NO: 16) or a variant thereof.

In other examples, the single domain antibody comprises the CDR1 that comprises the amino acid sequence of ASGRFFSSYA (SEQ ID NO: 5) or a variant thereof, the CDR2 that comprises the amino acid sequence of ISWSGGST (SEQ ID NO: 6) or a variant thereof and the CDR3 that comprises the amino acid sequence of AREGAYYPDSYYRTVRYD (SEQ ID NO: 7) or a variant thereof.

According to some examples, the single domain antibody comprises the CDR1 that comprises the amino acid sequence of ASGRTFSSY (SEQ ID NO: 8) or a variant thereof, the CDR2 that comprises the amino acid sequence of TGSGD (SEQ ID NO: 9) or a variant thereof and the CDR3 that comprises the amino acid sequence of YRTGGPPQW (SEQ ID NO: 10) or a variant thereof.

For various examples, the single domain antibody comprises the CDR1 that comprises the amino acid sequence of RTDSEST (SEQ ID NO: 11) or a variant thereof, the CDR2 that comprises the amino acid sequence of WRYA (SEQ ID NO: 12) or a variant thereof and the CDR3 that comprises the amino acid sequence of RYIYGSLSDSGSYDN (SEQ ID NO: 13) or a variant thereof.

Referring to some examples, the single domain antibody comprises the CDR1 that comprises the amino acid sequence of SGTIFSIDA (SEQ ID NO: 14) or a variant thereof, the CDR2 that comprises the amino acid sequence of QAPGKQRE (SEQ ID NO: 15) or a variant thereof and the CDR3 that comprises the amino acid sequence of AKPPTYYSLEPWGKGT (SEQ ID NO: 16) or a variant thereof.

Suitably, the single domain antibody comprises, consists of or consists essentially of an amino acid sequence selected from SEQ ID NOs: 1 to 4, or a fragment, variant or derivative thereof.

Suitably, the norovirus is of a GII genogroup. More particularly, the norovirus can be of a GII.4 genotype or a GII.17 genotype.

In some examples, the single domain antibody, in monovalent form, has a K_{D} for the P domain of the norovirus of lower than about 200 nM, lower than about 100 nM, lower than about 70 nM, lower than about 50 nM, lower than about 25 nM or lower than about 10 nM.

Suitably, the single domain antibody has been at least partly humanized.

In a second aspect, the present disclosure provides an antigen binding molecule comprising the single domain antibody of the first aspect.

Suitably, the antigen binding molecule is or comprises a monovalent single domain antibody, a multivalent single domain antibody, or a multispecific single domain antibody comprising one or more of the single domain antibodies of the first aspect. In some examples, the antigen binding molecule is a multispecific single domain antibody comprising a further single domain antibody that is directed to a P1 subdomain of a norovirus.

In some examples, the antigen binding molecule is or comprises an immunoconjugate. In this regard, the immunoconjugate may comprise one or more of a detectable marker, a therapeutic agent, a half-life extender and a nanocarrier.

In a third aspect, the present disclosure provides an isolated nucleic acid comprising a nucleotide sequence which encodes, or is complementary to a nucleotide sequence which encodes, the single domain antibody of the first aspect or the antigen binding molecule of the second aspect.

In particular examples, the isolated nucleic acid is or comprises mRNA.

In a fourth aspect, the present disclosure provides a genetic construct comprising: (i) the isolated nucleic acid of the third aspect; or (ii) a nucleotide sequence complementary thereto; operably linked or connected to one or more regulatory sequences in an expression vector.

In a fifth aspect, the present disclosure provides a host cell transformed with the nucleic acid molecule of the third aspect or the genetic construct of the fourth aspect.

In a sixth aspect, the present disclosure provides a method of producing the single domain antibody of the first aspect or the antigen binding molecule of the second aspect, including the steps of: (i) culturing the previously transformed host cell of the fifth aspect; and (ii) isolating the single domain antibody or the antigen binding molecule from said host cell cultured in step (i).

In a seventh aspect, the present disclosure provides a composition comprising the single domain antibody of the first aspect, the antigen binding molecule of the second aspect, the isolated nucleic acid of the third aspect, the genetic construct of the fourth aspect or the host cell of the fifth aspect and optionally a pharmaceutically acceptable carrier, diluent or excipient.

Suitably, the composition further comprises a further single domain antibody that is directed to a P1 subdomain of a norovirus.

In an eighth aspect, the present disclosure provides a method of diagnosing or monitoring a norovirus infection and/or a disease, disorder or condition associated therewith in a subject, said method including the step of contacting the subject and/or a biological sample from the subject with the single domain antibody of the first aspect, the antigen binding molecule of the second aspect or the composition of the seventh aspect.

Suitably, the present method further includes the step of detecting and/or measuring a level of antigen binding to the single domain antibody or the antigen binding molecule.

In a ninth aspect, the present disclosure provides a method of inhibiting or preventing binding of a norovirus to a histo-blood group antigen (HBGA) and/or a bile acid in a subject, said method including the step of administering to the subject a therapeutically effective amount of the single domain antibody of the first aspect, the antigen binding molecule of the second aspect, the isolated nucleic acid of the third aspect, the genetic construct of the fourth aspect, the host cell of the fifth aspect or the composition of the seventh aspect to thereby inhibit or prevent binding of the norovirus to the HBGA and/or the bile acid in the subject.

In a tenth aspect, the present disclosure provides a method of treating or preventing a norovirus infection and/or a disease, disorder or condition associated therewith in a subject including the step of administering a therapeutically effective amount of the single domain antibody of the first aspect, the antigen binding molecule of the second aspect, the isolated nucleic acid of the third aspect, the genetic construct of the fourth aspect, the host cell of the fifth aspect or the composition of the seventh aspect to thereby treat or prevent the norovirus infection and/or the disease, disorder or condition associated therewith in the subject.

In an eleventh aspect, the present disclosure provides for the use of the single domain antibody of the first aspect, the antigen binding molecule of the second aspect, the isolated nucleic acid of the third aspect, the genetic construct of the fourth aspect, the host cell of the fifth aspect or the composition of the seventh aspect for therapy.

In a twelfth aspect, the present disclosure provides for the use of the single domain antibody of the first aspect, the antigen binding molecule of the second aspect, the isolated nucleic acid of the third aspect, the genetic construct of the fourth aspect, the host cell of the fifth aspect or the composition of the seventh aspect in the manufacture of a medicament for the treatment and/or prevention of a norovirus infection and/or a disease, disorder or condition associated therewith in a subject.

In a thirteenth aspect, the present disclosure provides a method for inactivating or neutralising a norovirus associated with a substrate or surface, said method including the step of contacting the substrate or surface with an effective amount of the single domain antibody of the first aspect, the antigen binding molecule of the second aspect or the composition of the seventh aspect to thereby inactivate or neutralise the norovirus associated with the substrate or surface.

Suitably, the single domain antibody of the first aspect, the antigen binding molecule of the second aspect, the isolated nucleic acid of the third aspect, the genetic construct of the fourth aspect, the host cell of the fifth aspect or the composition of the seventh aspect are suitable for use in the methods of the eighth, ninth, tenth and/or thirteenth aspects.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

FIG. 1. Nanobody binding to VLPs. ELISA was performed to determine nanobody binding to immunization VLP antigens. All experiments were performed in triplicate and the standard deviation is shown. The cut-off value was set at OD₄₉₀ of 0.15 (dashed line). (A) GII.4 nanobodies binding to GII.4 Sydney-2012 VLPs, where NB-56 strongly bound to GII.4 VLPs at all dilutions, followed by NB-82, NB-53, and NB-30 and NB-76. (B) GIL 17 nanobodies binding to GII.17 Kawasaki308 VLPs, where NB-45 strongly bound to GII.17 VLPs at all dilutions, followed by NB-34, NB-2, and NB-7.

FIG. 2. Cross-reactivity of nanobodies. Nanobody cross-reactivities were analyzed using a panel of GI and GII noroviruses in direct ELISA. The results are colored on the heatmap schematic, where OD₄₉₂=3.0 for strong binding (red) and OD₄₉₂=0 for non-binding (white). (A) The GII.4 nanobodies were mainly GII.4 specific. NB-56 bound to five different GII.4 variants (i.e., GII.4c, CHDC, Saga, Yerseke, and Sydney-2012), whereas NB-30, NB-53, NB-76, and NB-82 all detected GII.4 Sydney-2012 VLPs and weakly detected several GII.4 variants. (B) NB-34 detected GII.1, GII.4 and GII.10 VLPs, whereas NB-7, NB-2 and NB-45 only detected GII.17 VLPs. Of note, for GII.17 nanobodies, only GII.4 Sydney-2012 VLPs were examined.

FIG. 3. Nanobody inhibition of VLP attachment to PGM. VLPs were pre-incubated with serially diluted nanobodies and added to PGM coated plates to analyze the ability for the nanobodies to block VLPs binding to HBGAs. (A) For the GII.4 nanobodies, NB-53 (IC₅₀=0.76 μg/mL), NB-56 (IC₅₀=0.26 μg/mL), and NB-76 (IC₅₀=1.417 μg/mL) showed a dose dependent inhibition, whereas NB-30 and NB-82 IC₅₀ values were not calculated, since the inhibition was below 50% for all dilutions. (B) For the GIL. 17 nanobodies, NB-45 showed the lowest IC₅₀ value (0.46 μg/mL), NB-2 (IC₅₀=0.84 g/mL), followed by NB-7 (IC₅₀=0.88 μg/mL), and NB-34 (IC₅₀=15.33 μg/mL).

FIG. 4. Thermodynamic properties of GII.4 nanobody binding to GII.4 Sydney-2012 P domain. The binding constants, K_{d} (dissociation constant, M), ΔH (heat change, cal/mole), ΔS (entropy change, cal/mole/deg), ΔG (change in free energy, cal/mol). Titrations were performed at 25° C. by injecting consecutive (2-3 μL) aliquots of nanobodies (100 μM) into GII.4 P domain (10-20 μM) in 120 s intervals. Examples of the titration (upper panels) of nanobodies to norovirus P domain are shown (A) NB-30, (B) NB-53, (C) NB-56, (D) NB-76, and (E) NB-82. The binding isotherm was calculated using a single binding site model (lower panels).

FIG. 5. Thermodynamic properties of GII.17 nanobody binding to GII.17 Kawasaki308 P domain. Titrations were performed at 25° C. by injecting consecutive (2-3 μL) aliquots of nanobodies (100 μM) into GII.17 P domain (10-20 μM) in 120 s intervals. Examples of the titration (upper panels) of nanobodies to norovirus P domain are shown (A) NB-2, (B) NB-7, (C) NB-34, and (D) NB-45. The binding isotherm was calculated using a single binding site model (lower panels).

FIG. 6. X-ray crystal structure of GII.4 Sydney-2012 P domain NB-30 complex. The X-ray crystal structure of the GII.4 P domain NB-30 complex was determined to 1.70 Å resolution. Molecular replacement indicated one P dimer and two NB-30 molecules in space group 1121. The GII.4 P domain monomers where colored accordingly (chain A: black and chain B; grey) and NB-30 (green). (A) NB-30 bound to the side of the P1 subdomain and involved a dimeric interaction with both P domain chains A and B with an interface area of ~1,182 Å². (B) A close-up view of the GII.4 P domain and NB-30 interacting residues (note, only main or side chains that are forming hydrogen bonds are shown for simplicity). The GII.4 P domain hydrogen bond interactions involved both side and main chain interactions. Electrostatic interactions were found between A chain: ASP-310^{GII.4} and ARG-110^{NB-30}; ARG-339^{GII.4} and GLU-115^{NB-30}; B chain: LYS-248^{GII.4} and ASP-101^{NB-30}; ASP-481^{GII.4} and ARG-54^{NB-30}. Based previous norovirus nanobodies (34, 35), the CDRs for NB-30 were approximately located CDR1 (26-33), CDR2 (51-58), and CDR3 (99-117).

FIG. 7. X-ray crystal structure of GII.4 Sydney-2012 P domain NB-53 complex. The GII.4 P domain NB-53 complex was determined to 2.30 Å resolution. Molecular replacement indicated one P dimer and two NB-53 molecules in space group 1222. (A) NB-53 (yellow) bound to the side of the P1 subdomain and involved a dimeric interaction with an interface area of ~859 Å². (B) A close-up view of GII.4 P domain and NB-53 interacting residues. The GII.4 P domain hydrogen bond interactions involved both side and main chain interactions. Electrostatic interactions were found between A chain: ARG-484^{GII.4} and ASP-27^{NB-53}, B chain: ASP-310^{GII.4} and HIS-59^{NB-53}, ASP-310^{GII.4} and LYS-64^{NB-53}. The CDRs for NB-53 were approximately located CDR1 (25-32), CDR2 (52-55), and CDR3 (98-104).

FIG. 8. X-ray crystal structure of GII.4 Sydney-2012 P domain NB-56 complex. The X-ray crystal structure of the GII.4 P domain NB-56 complex was determined to 1.60 Å resolution. Molecular replacement indicated two P dimers and four NB-56 molecules in space group P12₁1. (A) The NB-56 (salmon) bound to the side of the P1 subdomain and involved a dimeric interaction with an interface area of ~1,215 Å². (B) A close-up view of GII.4 P domain and NB-56 interacting residues. The GII.4 P domain hydrogen bond interactions involved both side and main chain interactions. An electrostatic interaction was found between chain B: ASP-289^{GII.4} and ARG-108^{NB-56}. The CDRs for NB-56 were approximately located CDR1 (26-32), CDR2 (52-55), and CDR3 (98-118).

FIG. 9. Binding site of NB-30, NB-53, and NB-56. Superposition of GII.4 P domain NB-30, GII.4 P domain NB-53, GII.4 P domain NB-56, GII.10 P domain Nano-32 (5003), GII.10 P domain A-trisaccharide (3PA1), and GII.4 P domain A1227 (6N8D) onto the GII.10 P domain (3ONU). NB-30 (green), NB-53 (yellow), NB-56 (salmon), Nano-32 (dark salmon), monoclonal antibody A1431 (light blue), and A-trisaccharide (blue) are shown on the GII. 10 P domain dimer (grey/black surface). Only one nanobody and monoclonal antibody are shown for clarity.

FIG. 10. X-ray crystal structure of GII.4 Sydney-2012 P domain NB-82 complex. The X-ray crystal structure of the GII.4 P domain NB-82 complex was determined to 1.70 Å resolution. Molecular replacement indicated three P dimers and six NB-82 molecules in space group P2₁2₁2₁. (A) The NB-82 (navy) bound to the side region of the P domain and involved a monomeric interaction with an interface area of ~832 Å². (B) A close-up view of GII.4 P domain and NB-82 interacting residues showing hydrogen bonds from both side and main chains. An electrostatic interaction was found between chain A: HIS-417^{GII.4} and ASP-104^{NB-82}. The CDRs for NB-82 were approximately located CDR1 (25-34), CDR2 (51-58), and CDR3 (100-103).

FIG. 11. X-ray crystal structure of GII.10 P domain NB-34 complex. The X-ray crystal structure of the complex was determined to 1.85 Å resolution. Molecular replacement indicated two P domains and two NB-34 molecules in space group P12₁1. (A) The NB-34 (cyan) bound to the side of the domain and involved a dimeric interaction with an interface area of ~1,017 Å². (B) A close-up view of GII.10 P domain and NB-34 interacting residues. The GII.10 P domain hydrogen bond interactions involved both side and main chain interactions. An electrostatic interaction was found between chain A: ASP-269^{GII.10} and ARG-103^{NB-34}; GLU-271^{GII.10} and ARG-103^{NB-34}. The CDRs for NB-34 were approximately located CDR1 (26-33), CDR2 (51-58), and CDR3 (99-107).

FIG. 12. Structural basis of NB-34 cross-reactivity. (A) Superposition of GII.10 P domain Nano-26 complex (5004, Nano-32 light magenta) and GII.4 P domain A1227 (6N81, monoclonal antibody A1227 purple) onto GII.10 Vietnam026 P domain NB-34 complex showing a highly similar binding region on the side of the P domain. (B) Superposition of GII.1 (4ROX, P domain forest), GII.4 (4OOS, P domain green), and GII.17 (5F4O, P domain tv green) P domain apo structures onto the GII.10 Vietnam026 P domain NB-34 complex (P domain grey). The P domain residues for GII.1, GII.4, and GII.17 at the identical location as GII.10 NB-34 binding residues are shown (GII.10 Vietnam026 VP1 numbering); NB-34 was removed for better viewing. NB-34 and Nano-26 bind a common set of seven P domain residues at this location, while amino acid substitutions among these genotypes were observed at only a four residues (underlined), i.e., ASP-320^{GII.10} (GLU-316^{GII.4}), GLU-271^{GII.10} (VAL-271^{GII.4}), SER-473^{GII.10} (ALA-465^{GII.4}), and GLU-489^{GII.10} (ASP-476^{GII.1}, ASP-481^{GII.4}, and ASP-481^{GII.17}); note the residue numbering among these genotypes varies.

FIG. 13. X-ray crystal structure of GII.4 Sydney-2012 P domain NB-76 complex. The X-ray crystal structure of the GII.4 P domain NB-76 complex was determined to 1.60 Å resolution. Molecular replacement indicated one P dimer and two NB-76 molecules in space group P2₁2₁2₁. (A) The NB-76 (gold) bound to the top of the P2 subdomain and involved a dimeric interaction with an interface area of ~945 Å². (B) A close-up view of GII.4 P domain and NB-76 interacting residues. The GII.4 P domain hydrogen bond interactions involved both side and main chain interactions. Electrostatic interactions were found between chain A: ASP-357^{GII.4} and GLN-1^{NB-76}; ASP-391^{GII.4} and ARG-45^{NB-76}. The CDRs for NB-76 were approximately located CDR1 (25-33), CDR2 (39-46), and CDR3 (98-113).

FIG. 14. Close-up of NB-76 blocking the GII.4 HBGA binding pocket. Superposition of the GII.4 Sydney-2012 P domain A-trisaccharide complex (4WZT) onto the GII.4 Sydney-2012 P domain NB-76 complex. The GII.4 Sydney-2012 P domain surface representation (black and grey), A-trisaccharide (blue sticks) are shown.

FIG. 15. Sequence and structural alignment of GII.4 variants (A) Sequence alignment of GII.4 variants showing NB-76 binding epitopes (gold) and A-trisaccharide binding residues (blue). Two HBGA binding epitopes engaged NB-76 (gold/blue shade). (B) Superposition of apo CHDC-1974 P domain (5IYN, green), apo Saga-2006 P domain (4OOX, orange), apo Sydney-2012 P domain (4OOS, red) onto Sydney-2012 P-NB76 complex (grey). NB-76 was omitted for clarity and residue numbering were Sydney-2012, while amino acid conservation and substitutions can be seen in FIG. 15A. The loop containing residues 391-398 was slightly shifted upon NB-76 binding.

FIG. 16. X-ray crystal structure of GII.17 Kawasaki308 P domain NB-2 complex. The X-ray crystal structure of the GII.17 P domain NB-2 complex was determined to 1.40 Å resolution. Molecular replacement indicated one P domain and one NB-2 molecule in space group C121. (A) NB-2 (orange) bound to the top of the P2 subdomain and involved a dimeric interaction with an interface area of ~756 Å². (B) A close-up view of the GII.17 P domain and NB-2 interacting residues. The GII.17 P domain hydrogen bond interactions involved both side and main chain interactions. An electrostatic interaction was found between chain A: ARG-297^{GII.17} and GLU-101^{NB-2}. The CDRs for NB-2 were approximately located CDR1 (24-33), CDR2 (51-58), and CDR3 (99-116).

FIG. 17. Close-up of NB-2 blocking the GII.17 HBGA binding pocket. Superposition of the GII.17 Kawasaki308 P domain A-trisaccharide complex (5LKC) onto the GII.17 Kawasaki308 P domain NB-2 complex. The GII.17 Kawasaki308 P domain surface representation (black and grey) and A-trisaccharide (blue sticks) are shown. The GII.17 P domain residues that typically bind HBGAs include THR-348, ARG-349, ASP-378, GLY-443, and TYR-444.

FIG. 18. X-ray crystal structure of GII.17 Kawasaki308 P domain NB-7 complex. The X-ray crystal structure of the GII.17 P domain NB-7 complex was determined to 2.99 Å resolution. Molecular replacement indicated one P domain and one NB-7 molecule in space group 1222 (only one NB-7 molecule was shown on the P domain dimer for clarity). (A) The NB-7 (lime green) bound to the top of the P2 subdomain and involved a dimeric interaction with an interface area of ~508 Å². (B) A close-up view of GII.17 P domain and NB-7 interacting residues. The GII.17 P domain hydrogen bond interactions involved both side and main chain interactions. The CDRs for NB-7 were approximately located CDR1 (24-32), CDR2 (52-56), and CDR3 (99-107).

FIG. 19. Close-up of NB-7 blocking the GII.17 HBGA binding site. Superposition of the GII. 17 Kawasaki308 P domain A-trisaccharide complex (5LKC) onto the GII.17 Kawasaki308 P domain NB-7 complex. The GII.17 Kawasaki308 P domain surface representation (black and grey) are shown.

FIG. 20. X-ray crystal structure of GII.17 Kawasaki308 P domain NB-45 complex. The X-ray crystal structure of the GII.17 domain NB-45 complex was determined to 2.10 Å resolution. Molecular replacement indicated one P dimer and two NB-45 molecules in space group P12₁1. (A) The NB-45 (hot pink) bound to the top of the P2 subdomain and involved a dimeric interaction with an interface area of ~1,147 Å². (B) A close-up view of GII.17 P domain and NB-45 interacting residues. The GII.17 P domain hydrogen bond interactions involved both side and main chain interactions. An electrostatic interaction was observed between chain A: ASP-396^{GII.17} and HIS-60^{NB-45}. The CDRs for NB-45 were approximately located CDR1 (27-33), CDR2 (53-56), and CDR3 (99-112).

FIG. 21. Close-up of NB-45 blocking the GII.17 HBGA site. Superposition of the GII.17 Kawasaki308 P domain A-trisaccharide complex (5LKC) onto the GII.17 Kawasaki308 P domain NB-45 complex. The GII.17 Kawasaki308 P domain surface representation (black and grey) and A-trisaccharide (blue sticks) are shown. An additional extended loop on NB-45 (residues 39-46) permitted this nanobody to completely overlap the HBGA pocket.

FIG. 22. Summary of GII norovirus nanobodies. A total of 16 different GII nanobodies were superimposed onto the GII.4 Sydney-2012 P dimer (3PA1 grey) to show nanobody binding sites. Note, only one nanobody per dimer is shown. This includes genotype specific and broadly reactive GII nanobodies (34, 35): Nano-4 GII.17 P domain (5O02), Nano-14 GII.10 P domain (5OMM), Nano-26 Nano-85 GII.10 P domain (5O04), Nano-27 GII.10 P domain (5OMN), Nano-32 GII.10 P domain (5003), Nano-42 GII.10 P domain (5005), and the nine newly characterized nanobodies in this study.

FIG. 23. Amino acid sequences for NB-2, NB-7, NB-45 and NB-76. CDR sequences are highlighted.

FIG. 24. Encoding nucleotide sequences for NB-2, NB-7, NB-45 and NB-76.

### KEY TO THE SEQUENCE LISTING

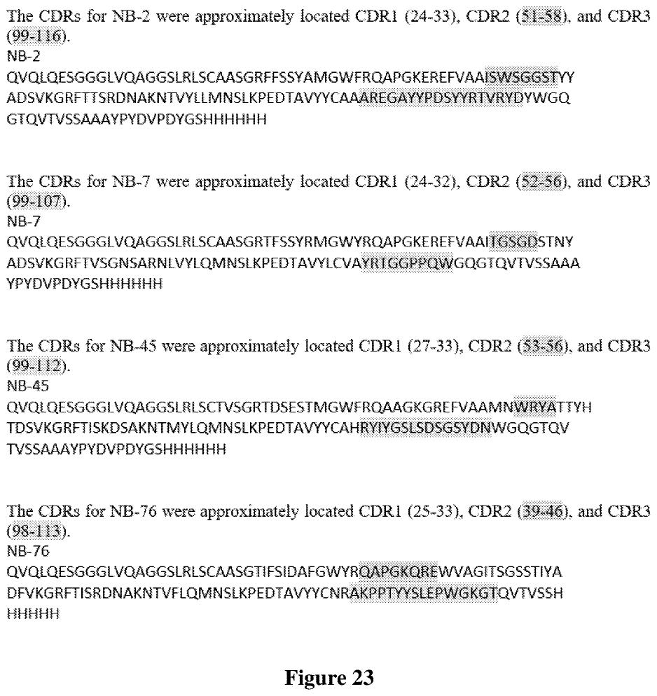

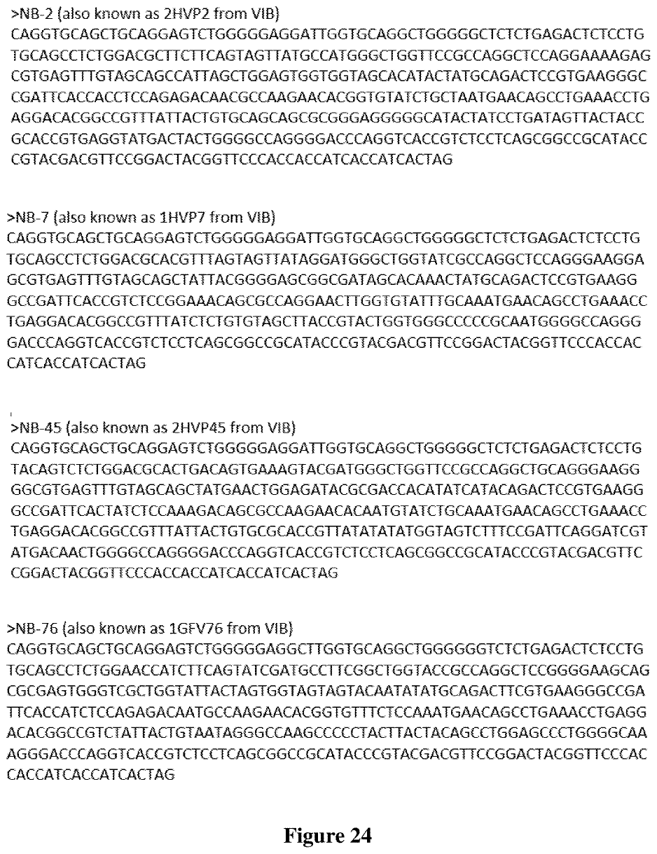

Key to the Sequence Listing

| SEQ ID NO: 1 | amino acid sequence of NB-2 single domain antibody of FIG. 23 |
|---|---|
| | QVQLQESGGGLVQAGGSLRLSCAASGRFFSSYAMGWFRQAPGKER |
| | EFVAAISWSGGSTYYADSVKGRFTTSRDNAKNTVYLLMNSLKPED |
| | TAVYYCAAAREGAYYPDSYYRTVRYDYWGQGTQVTVSSAAAYPY |
| | DVPDYGS |
| SEQ ID NO: 2 | amino acid sequence of NB-7 single domain antibody of FIG. 23 |
| | QVQLQESGGGLVQAGGSLRLSCAASGRTFSSYRMGWYRQAPGKE |
| | REFVAAITGSGDSTNYADSVKGRFTVSGNSARNLVYLQMNSLKPE |
| | DTAVYLCVAYRTGGPPQWGQGTQVTVSSAAAYPYDVPDYGS |
| SEQ ID NO: 3 | amino acid sequence of NB-45 single domain antibody of FIG. 23 |
| | QVQLQESGGGLVQAGGSLRLSCTVSGRTDSESTMGWFRQAAGKG |
| | REFVAAMNWRYATTYHTDSVKGRFTISKDSAKNTMYLQMNSLKP |
| | EDTAVYYCAHRYIYGSLSDSGSYDNWGQGTQVTVSSAAAYPYDVP |
| | DYGS |
| SEQ ID NO: 4 | amino acid sequence of NB-76 single domain antibody of FIG. 23 |
| | QVQLQESGGGLVQAGGSLRLSCAASGTIFSIDAFGWYRQAPGKQRE |
| | WVAGITSGSSTIYADFVKGRFTISRDNAKNTVFLQMNSLKPEDTAV |
| | YYCNRAKPPTYYSLEPWGKGTQVTVSS |
| SEQ ID NO: 5 | amino acid sequence of CDR1 of NB-2 single domain antibody of FIG. 23 |
| | ASGRFFSSYA |
| SEQ ID NO: 6 | amino acid sequence of CDR2 of NB-2 single domain antibody of FIG. 23 |
| | ISWSGGST |
| SEQ ID NO: 7 | amino acid sequence of CDR3 of NB-2 single domain antibody of FIG. 23 |
| | AREGAYYPDSYYRTVRYD |
| SEQ ID NO: 8 | amino acid sequence of CDR1 of NB-7 single domain antibody of FIG. 23 |
| | ASGRTFSSY |
| SEQ ID NO: 9 | amino acid sequence of CDR2 of NB-7 single domain antibody of FIG. 23 |
| | TGSGD |
| SEQ ID NO: 10 | amino acid sequence of CDR3 of NB-7 single domain antibody of FIG. 23 |
| | YRTGGPPQW |
| SEQ ID NO: 11 | amino acid sequence of CDR1 of NB-45 single domain antibody of FIG. 23 |
| | RTDSEST |
| SEQ ID NO: 12 | amino acid sequence of CDR2 of NB-45 single domain antibody of FIG. 23 |
| | WRYA |
| SEQ ID NO: 13 | amino acid sequence of CDR3 of NB-45 single domain antibody of FIG. 23 |
| | RYIYGSLSDSGSYDN |
| SEQ ID NO: 14 | amino acid sequence of CDR1 of NB-76 single domain antibody of FIG. 23 |
| | SGTIFSIDA |
| SEQ ID NO: 15 | amino acid sequence of CDR2 of NB-76 single domain antibody of FIG. 23 |
| | QAPGKQRE |
| SEQ ID NO: 16 | amino acid sequence of CDR3 of NB-76 single domain antibody of FIG. 23 |
| | AKPPTYYSLEPWGKGT |
| SEQ ID NO: 17 | nucleotide sequence encoding NB-2 single domain antibody of FIG. 24 |
| | CAGGTGCAGCTGCAGGAGTCTGGGGGAGGATTGGTGCAGGCTG |
| | GGGGCTCTCTGAGACTCTCCTGTGCAGCCTCTGGACGCTTCTTCA |
| | GTAGTTATGCCATGGGCTGGTTCCGCCAGGCTCCAGGAAAAGAG |
| | CGTGAGTTTGTAGCAGCCATTAGCTGGAGTGGTGGTAGCACATA |
| | CTATGCAGACTCCGTGAAGGGCCGATTCACCACCTCCAGAGACA |
| | ACGCCAAGAACACGGTGTATCTGCTAATGAACAGCCTGAAACCT |
| | GAGGACACGGCCGTTTATTACTGTGCAGCAGCGCGGGAGGGGG |
| | CATACTATCCTGATAGTTACTACCGCACCGTGAGGTATGACTACT |
| | GGGGCCAGGGGACCCAGGTCACCGTCTCCTCAGCGGCCGCATAC |
| | CCGTACGACGTTCCGGACTACGGTTCCCACCACCATCACCATCA |
| | CTAG |
| SEQ ID NO: 18 | nucleotide sequence encoding NB-7 single domain antibody of FIG. 24 |
| | CAGGTGCAGCTGCAGGAGTCTGGGGGAGGATTGGTGCAGGCTG |
| | GGGGCTCTCTGAGACTCTCCTGTGCAGCCTCTGGACGCACGTTTA |
| | GTAGTTATAGGATGGGCTGGTATCGCCAGGCTCCAGGGAAGGAG |
| | CGTGAGTTTGTAGCAGCTATTACGGGGAGCGGCGATAGCACAAA |
| | CTATGCAGACTCCGTGAAGGGCCGATTCACCGTCTCCGGAAACA |
| | GCGCCAGGAACTTGGTGTATTTGCAAATGAACAGCCTGAAACCT |
| | GAGGACACGGCCGTTTATCTCTGTGTAGCTTACCGTACTGGTGG |
| | GCCCCCGCAATGGGGCCAGGGGACCCAGGTCACCGTCTCCTCAG |
| | CGGCCGCATACCCGTACGACGTTCCGGACTACGGTTCCCACCAC |
| | CATCACCATCACTAG |
| SEQ ID NO: 19 | nucleotide sequence encoding NB-45 single domain antibody of FIG. 24 |
| | CAGGTGCAGCTGCAGGAGTCTGGGGGAGGATTGGTGCAGGCTG |
| | GGGGCTCTCTGAGACTCTCCTGTACAGTCTCTGGACGCACTGAC |
| | AGTGAAAGTACGATGGGCTGGTTCCGCCAGGCTGCAGGGAAGG |
| | GGCGTGAGTTTGTAGCAGCTATGAACTGGAGATACGCGACCACA |
| | TATCATACAGACTCCGTGAAGGGCCGATTCACTATCTCCAAAGA |
| | CAGCGCCAAGAACACAATGTATCTGCAAATGAACAGCCTGAAA |
| | CCTGAGGACACGGCCGTTTATTACTGTGCGCACCGTTATATATAT |
| | GGTAGTCTTTCCGATTCAGGATCGTATGACAACTGGGGCCAGGG |
| | GACCCAGGTCACCGTCTCCTCAGCGGCCGCATACCCGTACGACG |
| | TTCCGGACTACGGTTCCCACCACCATCACCATCACTAG |
| SEQ ID NO: 20 | nucleotide sequence encoding NB-76 single domain antibody of FIG. 24 |
| | CAGGTGCAGCTGCAGGAGTCTGGGGGAGGCTTGGTGCAGGCTG |
| | GGGGGTCTCTGAGACTCTCCTGTGCAGCCTCTGGAACCATCTTCA |
| | GTATCGATGCCTTCGGCTGGTACCGCCAGGCTCCGGGGAAGCAG |
| | CGCGAGTGGGTCGCTGGTATTACTAGTGGTAGTAGTACAATATA |
| | TGCAGACTTCGTGAAGGGCCGATTCACCATCTCCAGAGACAATG |
| | CCAAGAACACGGTGTTTCTCCAAATGAACAGCCTGAAACCTGAG |
| | GACACGGCCGTCTATTACTGTAATAGGGCCAAGCCCCCTACTTA |
| | CTACAGCCTGGAGCCCTGGGGCAAAGGGACCCAGGTCACCGTCT |
| | CCTCAGCGGCCGCATACCCGTACGACGTTCCGGACTACGGTTCC |
| | CACCACCATCACCATCACTAG |
| SEQ ID NO: 21 | amino acid sequence of a VP1 protein of norovirus strain GII.4 |
| | Sydney-2012 (JX459908) |
| | MKMASSDANPSDGSAANLVPEVNNEVMALEPVVGAAIAAPVAGQQNVIDPWIRNNFVQAP |
| | GGEFTVSPRNAPGEILWSAPLGPDLNPYLSHLARMYNGYAGGFEVQVILAGNAFTAGKVI |
| | FAAVPPNFPTEGLSPSQVTMFPHIVVDVRQLEPVLIPLPDVRNNFYHYNQSNDPTIKLIA |
| | MLYTPLRANNAGDDVFTVSCRVLTRPSPDFDFIFLVPPTVESRTKPFSVPVLTVEEMTNS |
| | RFPIPLEKLFTGPSSAFVVQPQNGRCTTDGVLLGTTQLSPVNICTFRGDVTHITGSRNYT |
| | MNLASQNWNDYDPTEEIPAPLGTPDFVGKIQGVLTQTTRTDGSTRGHKATVYTGSADFAP |
| | KLGRVQFETDTDRDFEANQNTKFTPVGVIQDGGTTHRNEPQQWVLPSYSGRNTHNVHLAP |
| | AVAPTFPGEQLLFFRSTMPGCSGYPNMDLDCLLPQEWVQYFYQEAAPAQSDVALLRFVNP |
| | DTGRVLFECKLHKSGYVTVAHTGQHDLVIPPNGYFRFDSWVNQFYTLAPMGNGTGRRRAV |
| SEQ ID NO: 22 | amino acid sequence of a VP1 protein of norovirus strain GII.10 Vietnam |
| | 026 (AF504671) |
| | MKMASNDAAPSNDGAAGLVPESNNEVMALEPVAGASLAAPVTGQTNIIDPWIRMNFVQAP |
| | NGEFTVSPRNSPGEVLLNLELGPELNPYLAHLSRMYNGYAGGMEVQIMLAGNAFTAGKLI |
| | FAAVPPHFPIENLSPPQITMFPHVIIDVRTLEPVLLPMPDIRNSFFHFIQRDEPKMRLVA |
| | MLYTPLRSNGSGDDVFTVSCRVLTRPTPDFDFTYLVPPTVESKSKPFTLPILTLGELTNS |
| | RFPLPIDVLYTNPNESAIVQCQNGRCTLDGELQGTTQLLPTGICAFRGKVTQQVQDEHRG |
| | THWNMTVTNLNGTPFDPTEDVPAPLGTPDFSGQIYGVISQRNTNTVPGEGNLPANRAHEA |
| | VIATYSPKFTPKLGNIQFSTWETQDVSSGQPTKFTPVGLASVDANSHFDQWTLPSYSGAL |
| | TLNMNLAPSVAPVFPGECLLFFRSFIPLKGGYGNPAIDCLMPQEWVQHLYQESAPSLSDV |
| | ALVRYVNPETGRTLFEAKLHRNGFLTVARNSAGPVVAPTNGYFRFDSWVNQFYTLAPMGN |
| | GSGRRRMQ |
| SEQ ID NO: 23 | amino acid sequence of a VP1 protein of norovirus strain GII.17 |
| | Kawasaki308 (LC037415) |
| | MKMASNDAAPSNDGAAGLVPEGNNETLPLEPVAGAAIAAPVTGONNIIDPWIRTNFVQAP |
| | NGEFTVSPRNSPGEILLNLELGPDLNPYLAHLSRMYNGYAGGVEVQVLLAGNAFTAGKIL |
| | FAAVPPNFPVEFLSPAQITMLPHLIVDVRTLEPIMIPLPDVRNTFFHYSNQPNSRMRLVA |
| | MLYTPLRSNGSGDDVFTVSCRVLTRPTPDFEFTYLVPPSVESKTKPFSLPILTLSELTNS |
| | RFPVPIDSLFTAQNNVLQVQCQNGRCTLDGELQGTTQLLPTGICAFRGRVTAQINQRDRW |
| | HMQLQNLNGTTYDPTDDVPAPLGTPDFKGVVFGMVSQRNVGNDAPGSTRAQQAWVSTYSP |
| | QFVPKLGSVNLRISDNDDFQFQPTKFTPVGVNDDDDGHPFRQWELPNYSGELTLNMNLAP |
| | PVAPNFPGEQLLFFRSFVPCSGGYNQGIIDCLIPQEWIQHFYQESAPSQSDVALIRYVNP |
| | DTGRTLFEAKLHRSGYITVAHSGDYPLVVPANGHFRFDSWVNQFYSLAPMGTGNGRRRAQ |
| SEQ ID NO: 24 | amino acid sequence of Hex-His linker |
| | HHHHHH |
| SEQ ID NO: 25 | VP1 epitope of nano-85 single domain antibody from WO2016059113 |
| | W-V-N-X¹-F-Y-X², wherein X¹ represents any amino acid, preferably Q or |
| | P, and X² represents any amino acid, preferably T or S |
| SEQ ID NO: 26 | amino acid sequence of CDR1 of nano-85 single domain antibody from |
| | WO2016059113 |
| | GSIFSIYA |
| SEQ ID NO: 27 | amino acid sequence of variant CDR1 of nano-85 single domain antibody |
| | from WO2016059113 |
| | GSIFSIYL |
| SEQ ID NO: 28 | amino acid sequence of CDR2 of nano-85 single domain antibody from |
| | WO2016059113 |
| | ISSGGGTN |
| SEQ ID NO: 29 | amino acid sequence of CDR3 of nano-85 single domain antibody from |
| | WO2016059113 |
| | KREDYSAYAPPSGS |
| SEQ ID NO: 30 | amino acid sequence of variant CDR3 of nano-85 single domain antibody |
| | from WO2016059113 |
| | KREDFSAYAPPSGS |
| SEQ ID NO: 31 | amino acid sequence of nano-85 single domain antibody from |
| | WO2016059113 |
| | Asp Val Gln Leu Val Glu Ser Gly Gly Gly Leu Val Gln Pro Gly Gly |
| | 1 5 10 15 |
| | Ser Leu Arg Leu Ser Cys Ala Ala Ser Gly Ser Ile Phe Ser Ile Tyr |
| | 20 25 30 |
| | Ala Met Gly Trp Tyr Arg Gln Ala Pro Gly Lys Gln Arg Glu Leu Val |
| | 35 40 45 |
| | Ala Ser Ile Ser Ser Gly Gly Gly Thr Asn Tyr Ala Asp Ser Val Lys |
| | 50 55 60 |
| | Gly Arg Phe Thr Ile Ser Gly Asp Asn Ala Lys Asn Thr Val Tyr Leu |
| | 65 70 75 80 |
| | Gln Met Asn Ser Leu Lys Pro Glu Asp Thr Ala Val Tyr Tyr Cys Lys |
| | 85 90 95 |
| | Arg Glu Asp Tyr Ser Ala Tyr Ala Pro Pro Ser Gly Ser Arg Gly Arg |
| | 100 105 110 |
| | Gly Thr Gln Val Thr Val Ser Ser Gly Arg Tyr Pro Tyr Asp Val Pro |
| | 115 120 125 |
| | Asp Tyr Gly Ser Gly Arg Ala |
| | 130 135 |
| SEQ ID NO: 32 | VP1 epitope of nano-26 single domain antibody from WO2019057755 |
| | a-x-a-h-x-h-x-o with “x” being any amino acid; “a” being glutamic acid or |
| | aspartic acid; “h” being glycine, alanine, valine, leucine or isoleucine, |
| | and “o” being serine or threonine |
| SEQ ID NO: 33 | VP1 epitope of nano-26 single domain antibody from WO2019057755 |
| | D-x-E-L-x-G-x-T with “x” being any amino acid |
| SEQ ID NO: 34 | VP1 epitope of nano-26 single domain antibody from WO2019057755 |
| | (N or Q)-(D or E)-(x)₁₅-P with “x” being any amino acid |
| SEQ ID NO: 35 | VP1 epitope of nano-26 single domain antibody from WO2019057755 |
| | Y-Q-E-S-x-P-(x)₁₂-P with “x” being any amino acid |
| SEQ ID NO: 36 | amino acid sequence of CDR1 of nano-26 single domain antibody from |
| | WO2019057755 |
| | RIIFFMYD |
| SEQ ID NO: 37 | amino acid sequence of CDR2 of nano-26 single domain antibody from |
| | WO2019057755 |
| | QINSDVST |
| SEQ ID NO: 38 | amino acid sequence of CDR3 of nano-26 single domain antibody from |
| | WO2019057755 |
| | YCNVRRASA |
| SEQ ID NO: 39 | amino acid sequence of nano-26 single domain antibody from |
| | WO2019057755 |
| | Gln Val Gln Leu Gln Glu Ser Gly Gly Gly Leu Val Gln Pro Gly Gly |
| | 1 5 10 15 |
| | Ser Leu Arg Leu Ser Cys Thr Ala Pro Arg Ile Ile Phe Phe Met Tyr |
| | 20 25 30 |
| | Asp Val Gly Trp Tyr Arg Gln Ala Pro Glu Lys Gln Arg Glu Leu Val |
| | 35 40 45 |
| | Ala Gln Ile Asn Ser Asp Val Ser Thr Lys Tyr Ala Asp Ser Val Lys |
| | 50 55 60 |
| | Gly Arg Phe Thr Ile Ser Arg Asp Asn Ala Lys Arg Thr Val Tyr Leu |
| | 65 70 75 80 |
| | Gln Met Asn Asp Leu Lys Pro Glu Asp Ala Ala Val Tyr Tyr Cys Asn |
| | 85 90 95 |
| | Val Arg Arg Ala Ser Ala Asp Tyr Trp Gly Gln Gly Thr Gln Val Thr |
| | 100 105 110 |
| | Val Ser Ser |
| | 115 |

### DETAILED DESCRIPTION

### General Techniques and Definitions

Unless specifically defined otherwise, all technical and scientific terms used herein shall be taken to have the same meaning as commonly understood by one of ordinary skill in the art (e.g., in genomics, immunology, molecular biology, immunohistochemistry, biochemistry, oncology, and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more particularly +/−5%, even more particularly +/−1%, of the designated value.

Throughout this specification, the word “comprise’ or variations such as “comprises” or “comprising” will be understood to imply the inclusion of a stated element, integer or step, or group of elements, integers or steps, but not the exclusion of any other element, integer or step, or group of elements, integers or steps.

By “consisting essentially of” in the context of an amino acid sequence, such as a VHH chain or CDR sequence, is meant the recited amino acid sequence together with an additional one, two or three amino acids at the N- or C-terminus thereof. By “consisting essentially of” in the context of a nucleotide sequence is meant the recited nucleotide sequence together with an additional one, two or three amino nucleic acids at the 5′ or 3′ end thereof.

All computer programs, algorithms, patent and scientific literature referred to herein is incorporated herein by reference. For the present disclosure, the database accession number or unique identifier provided herein for a gene or protein, as well as the gene and/or protein sequence or sequences associated therewith, are incorporated by reference herein.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### Single Domain Antibodies

The inventors have surprisingly shown for the first time that particular single domain antibodies derived from camelid-based heavy chain only antibodies (HCAbs) can selectively bind to or interact with a particular epitope (e.g., a P domain epitope, such as a P2 subdomain or a HBGA binding pocket thereof) on the P domain of a norovirus. These single domain antibodies were also demonstrated to potently inhibit or block binding of norovirus virus-like particles (VLPs) to histo-blood group antigens (HBGAs), host co-factors that are considered important for most norovirus infections, in in vitro assays.

Accordingly, the present disclosure provides a single domain antibody that is directed against a P domain, and more particularly a P2 subdomain, of a norovirus.

Even more particularly, the single domain antibody suitably binds to or interacts with one or more residues of a P2 subdomain. In some examples, the P2 subdomain comprises, consists of or consists essentially of residues 275 to 417 or residues 276 to 418 of a VP1 protein of the norovirus.

In a particular form, said single domain antibody comprises
(a) a CDR1 that comprises an amino acid sequence of ASGRFFSSYA (SEQ ID NO: 5), ASGRTFSSY (SEQ ID NO: 8), RTDSEST (SEQ ID NO: 11), SGTIFSIDA (SEQ ID NO: 14) or a variant thereof;
(b) a CDR2 that comprises an amino acid sequence of ISWSGGST (SEQ ID NO: 6), TGSGD (SEQ ID NO: 9), WRYA (SEQ ID NO: 12), QAPGKQRE (SEQ ID NO: 15) or a variant thereof; and
(c) a CDR3 that comprises an amino acid sequence of AREGAYYPDSYYRTVRYD (SEQ ID NO: 7), YRTGGPPQW (SEQ ID NO: 10), RYIYGSLSDSGSYDN (SEQ ID NO: 13), AKPPTYYSLEPWGKGT (SEQ ID NO: 16) or a variant thereof.

In the context of the present disclosure, the terms “single domain antibody”, “VHH”, “VHH antibody fragment”, “VHH chain” and “nanobody” can be used interchangeably herein and denote the variable domain or region of the single heavy chain of antibodies of the type of those found in camelids, which are naturally devoid of light chains. It is noted that the terms “nanobody” and “nanobodies” are registered trademarks of Ablynx N.V. and thus may also be referred to as Nanobody® and/or Nanobodies®.

In the absence of a light chain, single domain antibodies generally each have three CDRs, denoted CDR1, CDR2 and CDR3 respectively. Additionally, single domain antibodies typically include three or four framework regions (FRs; FR1, FR2, FR3 and optionally FR4). The single domain antibodies described herein can be derived from camel, dromedary, llama or alpaca HCAbs. In particular examples, the single domain antibodies according to the present disclosure are derived from alpaca HCAbs.

As used herein, “variable region” refers to the portions of the light and/or heavy chains of an antibody (e.g., the VHH chain of a camelid-derived antibody) as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. As used herein, the term “complementarity determining regions” (i.e., CDR1, CDR2, and CDR3) refers to the amino acid residues of an antibody variable region (e.g., a VHH chain) the presence of which are major contributors to specific antigen binding. Each VHH chain of a camelid-derived antibody typically has three CDR regions identified as CDR1, CDR2 and CDR3. “Framework regions” are those variable domain residues other than the CDR residues.

There are multiple conventions to define, annotate and describe the CDRs (and by extension FRs) of an immunoglobulin or antibody, such as a VHH chain or single domain antibody. To this end, the length and sequence of specific CDRs of an antibody can vary depending upon the specific nomenclature, algorithm or the like used to define them. Exemplary conventions to define CDRs include the Kabat definition (which is based on sequence variability and is the most commonly used; See, e.g., Sequences of Proteins of Immunological Interest, Kabat, et al.; National Institutes of Health, Bethesda, Md.; 5th ed.; NIH Publ. No. 91-3242 (1991)), the Chothia definition (which is based on the location of the structural loop regions; See, e.g., Chothia, et al., (1987) J Mol. Biol. 196:901-917), the AbM definition (which is a compromise between the Kabat and Chothia definitions and is based on Oxford Molecular's AbM antibody modelling software), the IMGT definition (see, e.g., https://www.imgt.org/IMGTindex/CDR.php) and the method described by Kontermann and Diibel (Eds., Antibody Engineering, vol 2, Springer Verlag Heidelberg Berlin, Martin, Chapter 3, pp. 33-51, 2010). In particular examples, the amino acid sequences of the CDR1, CDR2 and CDR3 of the single domain antibodies of the present disclosure are determined or defined by the Kabat definition. In other examples, the amino acid sequences of the CDR1, CDR2 and CDR3 of the single domain antibodies of the present disclosure are determined or defined by the Chothia definition. In some examples, the amino acid sequences of the CDR1, CDR2 and CDR3 of the single domain antibodies of the present disclosure are determined or defined by a crystal structure thereof.

Noroviruses belong to a genetically diverse group of non-enveloped, single-stranded RNA viruses of the Caliciviridae family. Human noroviruses have a single-stranded, positive sense RNA genome of ~7.7 kb. The norovirus genome generally contains three open reading frames (ORFs), where ORF1 encodes the non-structural proteins and includes the protease and RNA dependent RNA polymerase (RdRp), ORF2 encodes the capsid protein (VP1), and ORF3 encodes a small protein (VP2). The VP1 protein can be divided into two domains, a shell(S) domain and a protruding (P) domain, where the S domain surrounds the viral RNA, and the P domain, which can be further subdivided into P1 and P2 subdomains, contains the determinants for co-factor binding and antibody recognition (5). The P domain is generally in a dimeric form that protrudes from the shell of the norovirus capsid and engages HBGAs (typically two HBGA binding sites per P domain dimer), that are recognized co-factors and believed to be critical for most norovirus infections (6). Based on the ABH- and Lewis-HBGA types, at least nine different HBGA types have been found to interact with human noroviruses, although HBGA types and binding sites can vary among genogroups and genotypes (7-9).

For the present disclosure, it is envisaged that the term “norovirus” comprises any norovirus, irrespective of strain or origin. Suitably, the term “norovirus” encompasses those norovirus strains of Genogroup I, Genogroup II, Genogroup III, Genogroup IV, and Genogroup V (abbreviated as GI, GII, GIII, GIV or GV, respectively).

In certain examples, the norovirus provided herein comprises a norovirus strain selected from the group consisting of:
(i) Genogroup I genotype 1 (abbreviated as GI.1), GI.2, GI.3, GI.4, GI.5, GI.6, GI.7, GI.8, GI.9, GI.10, GI.11, GI.12, GI.13, GI.14, GI.15. GI.16 and/or GI.17;
(ii) Genogroup II genotype 1 (abbreviated as GII.1), GII.2, GII.3, GII.4, GII.5, GII.6, GII.7, GII.8, GII.9, GII.10, GII.11, GII.12, GII.13, GII.14, GII.15, GII.16, GII.17, GII.18, GII.19, GII.20, GII.21, GII.22, GII.23, and/or GII.24;
(iii) Genogroup III genotype 1 (abbreviated as GIII.1), GIII.2, GIII.3, and/or GIII.4;
(iv) Genogroup IV genotype 1 (abbreviated as GIV.1), GIV.2, GIV.3, and/or GIV.4;
(v) Genogroup V genotype 1 (abbreviated as GV.1), GV.2, GV.3, and/or GV.4;
and/or combinations of 2, 3, 4, 5, 6, 7, 8, 9, or 10, or more of any of the above noroviruses from different genogroups and/or different genotypes.

Suitably, the norovirus described herein is of a GII genogroup. More particularly, the norovirus suitably comprises a norovirus strain or genotype selected from the group consisting of a GII.4 norovirus, a GII.10 norovirus and a GII.17 norovirus. Even more particularly, the norovirus is suitably of a GII.4 genotype or a GII.17 genotype. According to various examples, the norovirus provided herein is of a GII.4 genotype, such as the GII.4 Sydney-2012 strain. In other examples, the norovirus provided herein is of a GII.10 genotype, such as the GII.10 Vietnam 026 strain. For various examples, the norovirus provided herein is of a GII.17 genotype, such as the GII.17 Kawasaki308 strain.

The terms “VP1”, “VP1 protein” or “capsid protein” as used herein includes any of the recombinant or naturally-occurring forms of a noroviral VP1 or capsid protein, or variants or homologs thereof that at least partly maintain or retain VP1 protein activity (e.g. within at least 50%, 80%, 90%, 95%, 96%, 97%, 98%, 99% or 100% activity compared to a wildtype or naturally occurring VP1 protein sequence). In some examples, the variants or homologs have at least 90%, 91%, 92%, 93%, 94%, 95%, 96%, 97%, 98%, 99% or 100% amino acid sequence identity across the whole sequence or a portion of the sequence (e.g. a 50, 100, 150, 200, 250, 300, 350, 400, 450 or 500 continuous amino acid portion), such as a P domain or P2 subdomain thereof, compared to a wildtype or naturally occurring VP1 protein sequence (e.g., SEQ ID NOs: 21 to 23). According to some examples, the VP1 protein is substantially identical to the protein identified by the UniProt accession number K4LM89 (norovirus strain GII.4 Sydney-2012), Q5F4T5 (Norwalk virus; norovirus strain GII.10 Vietnam 026) or A0A0E4B1P1 (norovirus strain GII.17 Kawasaki308).

Sequences of a VP1 protein for a range of norovirus strains are publicly available. Exemplary amino acid sequences are set forth in SEQ ID NOs: 21 to 23. Thus, the VP1 protein amino acid sequence may be a protein which is at least 70%, 75%, 80%, 85%, 90%, 95%, 96%, 97%, 98%, 99%, 99.5% or 100% identical to SEQ ID NO: 21 or a fragment or derivative thereof. Alternatively, the VP1 protein amino acid sequence may be a protein which is at least 70%, 75%, 80%, 85%, 90%, 95%, 96%, 97%, 98%, 99%, 99.5% or 100% identical to SEQ ID NO: 22 or a fragment or derivative thereof. In other examples, the VP1 protein amino acid sequence may be a protein which is at least 70%, 75%, 80%, 85%, 90%, 95%, 96%, 97%, 98%, 99%, 99.5% or 100% identical to SEQ ID NO: 23 or a fragment or derivative thereof.

The expression “directed against a P domain of a norovirus” is intended to mean that a single domain antibody or an antigen binding molecule of the present disclosure is capable of selectively or specifically binding to or interacting with and/or has been raised against an epitope of the P domain of a norovirus. Accordingly, the single domain antibody or antigen binding molecule may be described as specific or selective for the P domain of noroviruses, that is to say that it binds to the P domain of this virus to the exclusion of any other molecule or protein. In particular examples, the single domain antibody herein binds or interacts with one monomer of the P domain. In other examples, the single domain antibody herein binds or interacts with both monomers of the P domain.

Typically, a P domain of a VP1 protein of a norovirus comprises and extends from residue 222, 223, 224 or 225 thereof to the C-terminal end of said VP1 protein. In particular examples, a P domain comprises, consists of or consists essentially of residues 224 to 540 of a VP1 protein of a norovirus, such as a GII.4 strain of a norovirus. More particularly, a P domain may comprise, consist of or consist essentially of residues 224 to 540 of a VP1 protein having an amino acid sequence set forth in SEQ ID NO: 21. In certain examples, a P domain comprises, consists of or consists essentially of residues 222 to 548 or 549 of a VP1 protein of a norovirus, such as a GII.10 strain of a norovirus. More particularly, a P domain may comprise, consist of or consist essentially of residues 222 to 548 of a VP1 protein having an amino acid sequence set forth in SEQ ID NO: 22. In other examples, a P domain comprises, consists of or consists essentially of residues 225 to 540 of a VP1 protein of a norovirus, such as a GII.17 strain of a norovirus. More particularly, a P domain may comprise, consist of or consist essentially of residues 225 to 540 of a VP1 protein having an amino acid sequence set forth in SEQ ID NO: 23.

More particularly, the single domain antibody or antigen binding molecule disclosed herein may be directed against or bind to a P2 subdomain of a norovirus (i.e., is capable of selectively or specifically binding to or interacting with and/or has been raised against an epitope of the P2 subdomain thereof). To this end, the single domain antibody or antigen binding molecule may be described as specific or selective for a P2 subdomain of a VP1 protein of norovirus. In particular examples, a P2 subdomain comprises, consists of or consists essentially of residues 275 to 417 of a VP1 protein of a norovirus, such as a GII.4 strain of a norovirus. More particularly, a P2 subdomain may comprise, consist of or consist essentially of residues 275 to 417 of a VP1 protein having an amino acid sequence set forth in SEQ ID NO: 21. In certain examples, a P2 subdomain comprises, consists of or consists essentially of residues 278 to 426 of a VP1 protein of a norovirus, such as a GII.10 strain of a norovirus. More particularly, a P2 subdomain may comprise, consist of or consist essentially of residues 278 to 426 of a VP1 protein having an amino acid sequence set forth in SEQ ID NO: 22. In other examples, a P2 subdomain comprises, consists of or consists essentially of residues 276 to 418 of a VP1 protein of a norovirus, such as a GII.17 strain of a norovirus. More particularly, a P2 subdomain may comprise, consist of or consist essentially of residues 276 to 418 of a VP1 protein having an amino acid sequence set forth in SEQ ID NO: 23.

In certain examples, the single domain antibody binds to or interacts with one or more residues of a P domain selected from the group consisting of ASN-295, ARG-297, ARG-299, SER-374, ASP-395, ASP-396 and GLY-443. According to other examples, the single domain antibody binds to or interacts with one or more residues of a P domain selected from the group consisting of ARG-372, ASN-392, ASP-393, ASP-395, SER-441 and TYR-444. For certain examples, the single domain antibody binds to or interacts with one or more residues of a P domain selected from the group consisting of ASN-295, GLN-296, GLN-352, TRP-354, GLN-361, ARG-372, ASN-392, ASP-393, ASP-394, ASP-396, SER-441, GLY-442 and TYR-444. In relation to particular examples, the single domain antibody binds to or interacts with one or more residues of a P domain selected from the group consisting of LYS-329, THR-344, SER-355, ALA-356, ASP-357, GLU-368, ASP-391, THR-394, ASN-398, GLN-401 and GLY-443.

According to certain examples, the single domain antibody binds to or interacts with one or more residues of the P domain selected from the group consisting of:
(a) LYS-329, THR-344, SER-355, ALA-356, ASP-357, GLU-368, ASP-391, THR-394, ASN-398, GLN-401 and GLY-443 according to the amino acid numbering of a VP1 protein of a GII.4 genotype (e.g., SEQ ID NO: 21); and/or
(b) ASN-295, GLN-296, ARG-297, ARG-299, GLN-352, TRP-354, GLN-361, ARG-372, SER-374, ASN-392, ASP-393, ASP-394, ASP-395, ASP-396, SER-441, GLY-442, GLY-443 and TYR-444 according to the amino acid numbering of a VP1 protein of a GII. 17 genotype (e.g., SEQ ID NO: 23).

Suitably, the single domain antibody of the present disclosure binds to or interacts with one or more residues of a P2 subdomain selected from the group consisting of:
(a) LYS-329, THR-344, SER-355, ALA-356, ASP-357, GLU-368, ASP-391, THR-394, ASN-398 and GLN-401 according to the amino acid numbering of a VP1 protein of a GII.4 genotype (e.g., SEQ ID NO: 21); and/or
(b) ASN-295, GLN-296, ARG-297, ARG-299, GLN-352, TRP-354, GLN-361, ARG-372, SER-374, ASN-392, ASP-393, ASP-394, ASP-395 and ASP-396 according to the amino acid numbering of a VP1 protein of a GII.17 genotype (e.g., SEQ ID NO: 23).

In certain examples, the single domain antibody binds to or interacts with one or more residues of a P2 subdomain selected from the group consisting of ASN-295, ARG-297, ARG-299, SER-374, ASP-395 and ASP-396. According to other examples, the single domain antibody binds to or interacts with one or more residues of a P2 subdomain selected from the group consisting of ARG-372, ASN-392, ASP-393 and ASP-395. For certain examples, the single domain antibody binds to or interacts with one or more residues of a P2 subdomain selected from the group consisting of ASN-295, GLN-296, GLN-352, TRP-354, GLN-361, ARG-372, ASN-392, ASP-393, ASP-394 and ASP-396. In relation to particular examples, the single domain antibody binds to or interacts with one or more residues of a P2 subdomain selected from the group consisting of LYS-329, THR-344, SER-355, ALA-356, ASP-357, GLU-368, ASP-391, THR-394, ASN-398 and GLN-401.

For such examples, the single domain antibody may further bind to or interact with one or more residues of a P1 subdomain. For GII.4 norovirus strains, the P1 subdomain may comprise, consist of or consist essentially of residues 224 to 274 and 418 to 530 of a VP1 protein, such as that set forth in SEQ ID NO: 21. For GII.10 norovirus strains, the P1 subdomain may comprise, consist of or consist essentially of residues 222 to 277 and 427 to 548 or 549 of a VP1 protein, such as that set forth in SEQ ID NO: 22. For GII.17 norovirus strains, the P1 subdomain may comprise, consist of or consist essentially of residues 225 to 275 and 419 to 540 of a VP1 protein, such as that set forth in SEQ ID NO: 23.

Suitably, the single domain antibody may further bind to or interact with one or more residues of an interface loop domain of a P1 subdomain (i.e., in addition to one or more residues of the P2 subdomain). It is known in the art that the interface loop domain of the P1 subdomain can vary in length between respective norovirus genogroups, genotypes and strains. In some examples, however, the interface loop domain comprises, consists of or consists essentially of residues 420 to 470, 430 to 460, 440 to 450, 440 to 445 or 441 to 444, inclusive of any range therein, of a VP1 protein, such as that set forth in SEQ ID NOs: 21 to 23. More particularly, the single domain antibody may further bind to or interact with one or more residues of a P1 subdomain selected from GLY-443 according to the amino acid numbering of a VP1 protein of a GII.4 genotype (e.g., SEQ ID NO: 21); and/or SER-441, GLY-442, GLY-443 and TYR-444 according to the amino acid numbering of a VP1 protein of a GII.17 genotype (e.g., SEQ ID NO: 23). In alternative examples, the single domain antibody or antigen binding molecule of the present disclosure does not substantially bind or interact with a P1 subdomain of a VP1 protein a norovirus.

Suitably, the single domain antibody substantially binds to only one monomer of the P domain. In certain examples, the single domain antibody binds to or interacts with one or more residues of a first monomer of the P domain selected from the group consisting of ASN-295, ARG-297, ARG-299, SER-374, ASP-395, ASP-396, and GLY-443. According to other examples, the single domain antibody binds to or interacts with one or more residues of a first monomer of the P domain selected from the group consisting of ARG-372, ASN-392, ASP-393, ASP-395, SER-441, and TYR-444.

In alternative examples in which the single domain antibody provided herein binds to both monomers of the P domain, the single domain antibody may bind to one or more residues of a first monomer of the P domain and one or more residues of a second monomer of the P domain. Suitably, the one or more residues on the first monomer to which the single domain antibody of the present disclosure binds or interacts with are different to those on the second monomer to which the single domain antibody also binds or interacts with.

In certain examples, the single domain antibody binds to or interacts with one or more residues of a first monomer of the P domain selected from the group consisting of GLN-352, TRP-354, ARG-372, ASN-392, ASP-393, ASP-394, ASP-396, SER-441, GLY-442, and TYR-444 and one or more residues of a second monomer of the P domain selected from the group consisting of ASN-295, GLN-296, and GLN-361. According to other examples, the single domain antibody binds to or interacts with one or more residues of a first monomer of the P domain selected from the group consisting of LYS-329, SER-355, ALA-356, ASP-357, GLU-368, ASP-391, THR-394, ASN-398, GLN-401 and GLY-443 and one or more residues of a second monomer of the P domain comprising or consisting of THR-344.

It is contemplated that residue or amino acid numbering herein can be based on any full length sequence of a VP1 protein of a norovirus known in the art (e.g., an amino acid sequence set forth in any one of SEQ ID NOs: 21-23; i.e., the amino acid numbering is based on the first methionine (methionine in position 1; MET-1) being the first residue). In particular examples, residue numbering is based on a VP1 protein that is derived from a GII.4 strain (e.g., SEQ ID NO: 21). According to other examples, residue numbering is based on a VP1 protein that is derived from a GII.10 strain (e.g., SEQ ID NO: 22). For certain examples, residue numbering is based on a VP1 protein that is derived from a GII.17 strain (e.g., SEQ ID NO: 23).

Given the sequence similarity between the respective VP1 proteins and P domains of different noroviral genogroups, genotypes and strains, the single domain antibody or antigen binding molecule of the present disclosure may also be capable of selectively or specifically binding to a P domain, and more particularly a P2 subdomain, of a VP1 protein of a heterologous genogroup, genotype and/or strain of a norovirus (e.g., a genotype, genogroup or strain of norovirus that is different to that genotype, genogroup or strain from which the P domain of the VP1 protein has been utilized to raise or generate the single domain antibody in question). Accordingly, in some examples, the single domain antibodies and antigen binding molecules of the present disclosure may be able to prevent or treat noroviral infections associated with one or more other genogroups, genotypes, variants and/or strains of norovirus in addition to that genogroup, genotype, variant and/or strain of norovirus from which the single domain antibody in question has been raised or generated.

In view of the above, the single domain antibody or the antigen binding molecule provided herein can specifically or selectively bind to a P domain, such as a P2 subdomain thereof, of a VP1 protein of a norovirus. The terms “specifically binds” or “selectively binds” can be used interchangeably herein and shall be taken to mean that the binding interaction between a binding agent disclosed herein (e.g., a single domain antibody, an antigen binding molecule) and a target molecule described herein (e.g., VP1 protein, such as those set forth in SEQ ID NOs: 21 to 23) is dependent on detection of the target molecule by the binding agent. Accordingly, the binding molecule preferentially binds or recognizes the target molecule even when present in a mixture of other molecules or organisms. The formation of a complex with a target molecule (e.g., a VP1 protein) that is relatively stable under physiologic conditions can also be a characteristic of such selective or specific binding by a VHH chain of a single domain antibody or an antigen binding molecule.

As used herein, the term “binds” refers to the interaction (e.g., the formation of hydrogen bonds) of a binding agent, such as a single domain antibody or an antigen binding molecule, with a target molecule (e.g., a VP1 or capsid protein) and means that the interaction is dependent upon the presence of a particular structure (e.g., an antigenic determinant or epitope) on the target molecule. For example, a single domain antibody or an antigen binding molecule of the present disclosure recognizes and binds to a specific structural element (e.g., a P domain) of a VP1 protein of a norovirus rather than to molecules generally.

Specific or selective binding can be characterized by a K_{D} of about 5×10⁻²M or less (e.g., less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of a single domain antibody or an antigen binding molecule (e.g., a multi-specific antigen binding molecule) to a target molecule or an effector molecule are well known in the art and include, for example, equilibrium dialysis, surface plasmon resonance (SPR; e.g., Biacore assays), fluorescent-activated cell sorting (FACS) binding assays and the like.

According to certain examples, the single domain antibody binds to a P domain, and more particularly a P2 subdomain, of a VP1 protein of a norovirus with high affinity or relatively high affinity. As used herein, the terms “high affinity” and “relatively high affinity” are used interchangeably herein and refer to a binding affinity between a binding agent and the target molecule of interest with a K_{D} of at least about 10⁻⁶ M, more particularly at least about 10⁻⁷ M, even more particularly at least about 10⁻⁷ M and still even more particularly between about 10⁻⁸ M to about 10⁻¹⁰ M. Again, the determination of such affinity may be conducted under standard competitive binding immunoassay procedures, such as those provided herein.

In particular examples, the single domain antibody provided herein, or a conjugate thereof, in a monovalent format, has a K_{D} for the P domain of a VP1 protein of a norovirus virus (e.g., SEQ ID NOs: 21 to 23) of lower than about 600 nM (e.g., lower than about 600, 550, 500, 450, 400, 350, 300, 250, 200, 150, 100, 50, 40, 30, 20, 10, 5 nM or any range therein), lower than about 300 nM, lower than about 150 nM, lower than about 100 nM, lower than about 50 nM, lower than about 25 nM, lower than about 10 nM or lower than about 5 nM. More particularly, the single domain antibody, in monovalent form, suitably has a K_{D} for the P domain of the norovirus of lower than about 200 nM, lower than about 100 nM, lower than about 70 nM, lower than about 50 nM, lower than about 25 nM or lower than about 10 nM.

According to certain examples, the single domain antibody or antigen binding molecule provided herein does not significantly or substantially bind to a molecule other than the target molecule (e.g., a VP1 protein of a norovirus). The phrase “does not significantly bind to” or “does not substantially bind to” can mean, for example, that the single domain antibody or antigen binding molecule provided herein binds to a molecule other than the target molecule (or to any molecule other than the target molecule) with a binding affinity (e.g., K_{D}) that is at most 50% (e.g., 50%, 45%, 40%, 35%, 30%, 25%, 20%, 15%, 10%, 5%, 1% or less or any range therein) of the binding affinity of said single domain antibody or antigen binding molecule for the target molecule, such as a VP1 protein expressed by a norovirus, under the same physiological conditions.

The current disclosure describes a single domain antibody that selectively binds to a norovirus P domain epitope, and more particularly a P2 subdomain epitope. According to various examples, the single domain antibody or antigen binding molecule provided herein inhibits, ameliorates, treats or prevents a norovirus virus infection by at least partly inhibiting, disrupting or preventing binding or interaction of norovirus viral particles with host-derived co-factors, such as HBGA and bile acids, which are necessary for virus-host cell membrane fusion.

As such, the single domain antibodies or the antigen binding molecules of the present disclosure are capable of neutralizing a norovirus and/or blocking HBGA and/or bile acid binding thereto in a virus neutralization assay. In particular examples, the single domain antibodies (e.g., in a monovalent or bivalent format) or the antigen binding molecules are capable of neutralizing a norovirus or blocking HBGA binding thereto in a neutralization assay at an IC₅₀ of less than about 10 μg/mL (e.g., less than about 10, 9.5, 9.0, 8.5, 8.0, 7.5, 7.0, 6.5, 6.0, 5.5, 5.0, 4.5, 4.0, 3.5, 3.0, 2.5, 2.0, 1.5, 1, 0.5, 0.1, 0.05, 0.01 μg/mL or any range therein), less than about 5 μg/mL, less than about 4 μg/mL, less than about 3 μg/mL, less than about 2 μg/mL, less than about 1 μg/mL or less than about 0.5 μg/mL. Suitable virus neutralisation assays include, for example, HBGA blocking assays, such as those described herein, plaque reduction assays, pseudovirus neutralisation assays and microneutralisation assays.

Suitably, the single domain antibody includes the CDR1 that comprises the amino acid sequence of ASGRFFSSYA (SEQ ID NO: 5) or a variant thereof, the CDR2 that comprises the amino acid sequence of ISWSGGST (SEQ ID NO: 6) or a variant thereof and the CDR3 that comprises the amino acid sequence of AREGAYYPDSYYRTVRYD (SEQ ID NO: 7) or a variant thereof. In these examples, the single domain antibody suitably comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1.

In other examples, the single domain antibody includes the CDR1 that comprises the amino acid sequence of ASGRTFSSY (SEQ ID NO: 8) or a variant thereof, the CDR2 that comprises the amino acid sequence of TGSGD (SEQ ID NO: 9) or a variant thereof and the CDR3 that comprises the amino acid sequence of YRTGGPPQW (SEQ ID NO: 10) or a variant thereof. For such examples, the single domain antibody suitably comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 2.

According to various examples, the single domain antibody includes the CDR1 that comprises the amino acid sequence of RTDSEST (SEQ ID NO: 11) or a variant thereof, the CDR2 that comprises the amino acid sequence of WRYA (SEQ ID NO: 12) or a variant thereof and the CDR3 that comprises the amino acid sequence of RYIYGSLSDSGSYDN (SEQ ID NO: 13) or a variant thereof. In relation to these examples, the single domain antibody suitably comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 3.

Referring to certain examples, the single domain antibody includes the CDR1 that comprises the amino acid sequence of SGTIFSIDA (SEQ ID NO: 14) or a variant thereof, the CDR2 that comprises the amino acid sequence of QAPGKQRE (SEQ ID NO: 15) or a variant thereof and the CDR3 that comprises the amino acid sequence of AKPPTYYSLEPWGKGT (SEQ ID NO: 16) or a variant thereof. For these examples, the single domain antibody suitably comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 4.

Suitably, the single domain antibody disclosed herein, comprises, consists of or consists essentially of an amino acid sequence selected from SEQ ID NOs: 1 to 4, or a fragment, variant or derivative thereof. In some examples, the single domain antibody comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1. For other examples, the single domain antibody comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 2. According to certain examples, the single domain antibody comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 3. In various examples, the single domain antibody comprises, consists of or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 4.

The single domain antibodies, antigen binding molecules, nucleic acids, genetic constructs and host cells described herein may be considered to be isolated. For the purposes of the present disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids (e.g., no more than 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids and any range therein) and “polypeptide”, which is typically used to describe a protein having more than fifty (50) amino acids.

As used herein, a protein, polypeptide or peptide “variant” shares a definable amino acid sequence relationship with a reference amino acid sequence. In particular examples, the reference amino acid sequence is that of a CDR1, CDR2 or CDR3 sequence. As such, the reference amino acid sequence may be the amino acid sequence of any one of SEQ ID NOs: 5 to 16. According to other examples, the reference amino acid sequence is that of a VHH chain sequence, such as that of any one of SEQ ID NOs: 1-4. For some examples, the reference amino acid sequence is that of a FR1, FR2, FR3 or FR4 sequence.

The “variant” protein, polypeptide or peptide may have one or a plurality of amino acids of the reference amino acid sequence deleted, inserted/added or substituted by different amino acids. It is well understood in the art that some amino acids may be substituted, inserted/added or deleted without changing the activity of the single domain antibody (i.e., conservative substitutions). Accordingly, one or more of the residues of a single domain antibody, such as of a CDR (e.g., those defined by SEQ ID NOs: 5-16) and/or a FR, may be conservatively modified (e.g., by amino acid substitution or deletion) without altering the biological activity, function, or other desired property of the single domain antibody, such as its affinity or its specificity for an antigen like a P domain of a VP1 protein.

In some examples, a variant of the single domain antibody provided herein substantially retains the antigen binding ability (i.e., VP1 protein, and more particularly, P2 subdomain thereof, binding ability) of the unmodified or reference single domain antibody. Thus, one or more amino acid residues within the CDR and/or FR regions of a single domain antibody of the present disclosure can be deleted or replaced with other amino acid residues, such as those from the same side chain family, and the variant single domain antibody can be tested for retained function (e.g., the ability to specifically bind a P domain or P2 subdomain of a VP1 protein of a norovirus at high affinity) using the functional assays described herein.

According to some examples, modifications can be made to decrease the immunogenicity of the single domain antibody. For example, one approach is to modify one or more FR residues to that respective FR residue of the corresponding human germline sequence. Another type of framework modification involves modifying one or more residues within the FR and/or CDR regions to remove T cell epitopes to thereby reduce the potential immunogenicity of the single domain antibody.

Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent can be found in, for example, Bowie et al., Science 247:1306-1310 (1990).

Suitably, protein, polypeptide or peptide variants provided herein share at least 70% or 75%, more particularly at least 80% or 85% or even more particularly at least 90%, 91%, 92%, 93%, 94%, 95%, 96%, 97%, 98% or 99% sequence identity with a reference amino acid sequence, such as those set forth in SEQ ID NOs: 1-16 and 21-39. To this end, variants of the single domain antibodies described herein and their respective CDRs are contemplated for the present disclosure.

Accordingly, modifications to the CDR sequences disclosed herein are envisaged. In particular examples, said CDR1 comprises, consists essentially of or consists of the amino acid sequence of SEQ ID NO. 5 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In other examples, said CDR1 comprises, consists essentially of or consists of the amino acid sequence of SEQ ID NO. 8 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In some examples, said CDR1 comprises, consists essentially of or consists of the amino acid sequence of SEQ ID NO. 11 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In various examples, said CDR1 comprises, consists essentially of or consists of the amino acid sequence of SEQ ID NO. 14 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto.

In certain examples, said CDR2 comprises, consists essentially of or consists of the amino acid sequence of SEQ ID NO. 6 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In particular examples, said CDR2 comprises, consists essentially of or consists of the amino acid sequence of SEQ ID NO. 9 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In various examples, said CDR2 comprises, consists essentially of or consists of the amino acid sequence of SEQ ID NO. 12 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In other examples, said CDR2 comprises, consists essentially of or consists of the amino acid sequence of SEQ ID NO. 15 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto.

According to further examples, said CDR3 comprises, consists essentially of or consists of the amino acid sequence of SEQ ID NO. 7 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. For some examples, said CDR3 comprises, consists essentially of or consists of the amino acid sequence of SEQ ID NO. 10 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In other examples, said CDR3 comprises, consists essentially of or consists of the amino acid sequence of SEQ ID NO. 13 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto. In various examples, said CDR3 comprises, consists essentially of or consists of the amino acid sequence of SEQ ID NO. 16 or a sequence that is at least 80%, 81%, 82%, 83%, 84%, 85%, 86%, 87%, 88%, 89%, 90%, 91%, 92%, 93%, 94%, 95% 96%, 97%, 98% or 99% identical thereto.

Larger peptides and isolated proteins comprising a plurality of single domain antibodies (e.g., multivalent or multispecific antigen binding molecules) or conjugates thereof are also contemplated by the present disclosure and are described in more detail hereinafter.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of, for example, 6, 9, 12 or 20 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1991, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) or amino acid residue occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

The present disclosure also provides fragments of the single domain antibodies. As used herein, a “fragment” is a segment, domain, portion or region of a protein or peptide (such as those set forth in SEQ ID NOs: 1-4) which constitutes less than 100% of the amino acid sequence of the protein or peptide.

In general, fragments may comprise, consist essentially of or consist of up to 50, 55, 60, 65, 70, 75, 80, 85, 90, 95, 100, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139 or 140 contiguous amino acids of a single domain antibody (such as of SEQ ID NOs: 1-4). In a particular example, the protein fragment is or comprises a conserved region or one or more conserved amino acids, such as the CDR1-CDR3, of a single domain antibody. In this regard, one or more residues of FR1 and/or FR4 at an N- and/or C-terminus of a single domain antibody may not be present in the protein fragment. In various examples, the fragment comprises, or is contained within, a single domain antibody, such as those set forth in SEQ ID NOs: 1-4.

Suitably, the fragment substantially retains the antigenic binding ability of the single domain antibody from which the fragment is derived. In this regard, fragments of the present disclosure may retain the CDR1, CDR2 and CDR3 sequences of the single domain antibody. Additionally, fragments of the present disclosure suitably retain at least partly a natural structure and/or conformation of the full length peptide or protein.

The present disclosure also contemplates derivatives of the single domain antibodies described herein. As used herein, “derivatives” are molecules such as proteins, fragments or variants thereof that have been altered, for example, by conjugation or complexing with other chemical moieties, by post-translational modification (e.g., phosphorylation, acetylation and the like), modification of glycosylation (e.g., adding, removing or altering glycosylation), lipidation and/or inclusion of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), fluorescent protein sequences (e.g., GFP), polylysine, epitope tags, such as myc, FLAG and haemagglutinin tags. In one particular example, an additional amino acid sequence may comprise one or a plurality of histidine residues at an N and/or C-terminus thereof (e.g., hexa-histidine; SEQ ID NO: 24). The plurality of histidine residues (e.g., polyhistidine) may be a linear sequence of histidine residues or may be branched chain sequences of histidine residues. These additional histidine residues may facilitate purification of the single domain antibody.

Other derivatives contemplated by the disclosure include, but are not limited to, modification to side chains, incorporation of unnatural amino acids and/or their derivatives during peptide, or protein synthesis and the use of crosslinkers and other methods which impose conformational constraints on the single domain antibodies, fragments and variants of the disclosure. In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

Further derivatives may include conjugates of the single domain antibodies. The term “conjugated” can be used in the context of the present disclosure to describe single domain antibodies disclosed herein that are conjugated to another compound or structure, such as a label or carrier molecule or protein. Accordingly, in one example, the single domain antibodies of the present disclosure are “conjugated”. Single domain antibodies of the disclosure may be modified via conjugation or complexing with other chemical moieties, by post-translational modification (e.g., phosphorylation, ubiquitination, glycosylation), chemical modification (e.g., cross-linking, acetylation, biotinylation, oxidation or reduction) and/or conjugation with labels (e.g., fluorophores, enzymes, radioactive isotopes) and/or other functional elements (e.g., a half-life extender, a therapeutic agent), as described in more detail below in relation to antigen binding molecules.

The present disclosure also contemplates “naked” single domain antibodies. The term “naked” can be used to describe single domain antibodies of the present disclosure that are not conjugated to another compound or incorporated into a broader structure. Put another way, the single domain antibodies of the present disclosure can be unconjugated.

Conjugated single domain antibodies of the disclosure suitably retain their ability to bind a P domain, and more particularly a P2 subdomain, of a VP1 protein of a norovirus. In an example, a single domain antibody disclosed herein is conjugated to a label, such as biotin, so as to facilitate coupling to a substrate. Additional C- or N-terminal residues may be used as linkers to conjugate the single domain antibodies of the present disclosure to another moiety, or tags that aid the detection of the molecule. Such linkers and tags are well known in the art and include, for example, linker His tags, e.g., hexa-His (HHHHHH, SEQ ID NO: 24) or myc tags.

In various examples, the single domain antibody is conjugated to an Fc region or Fc domain, such as by way of a linker. The Fc domain or Fc region may be useful for extending the half-life of the single domain antibody, or otherwise to provide a desired functionality conferred by the Fc domain or Fc region, such as recognition by a secondary reagent or binding to a solid support for purification.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. By “Fc domain” as used herein is meant a polypeptide that comprises all or part of an Fc region.

The single domain antibodies of the present disclosure, inclusive of variants, fragments and/or derivatives thereof, may be produced by any means known in the art, including but not limited to, chemical synthesis, recombinant DNA technology and proteolytic cleavage to produce peptide fragments.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al, (John Wiley & Sons, Inc. NY USA 1995-2008). In this regard, reference is also made to International Publication WO 99/02550 and International Publication WO 97/45444.

Recombinant proteins may be conveniently prepared by a person skilled in the art using standard protocols as for example described in Sambrook et al, MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al, (John Wiley & Sons, Inc. NY USA 1995-2008), in particular Chapters 10 and 16; and CURRENT PROTOCOLS IN PROTEIN SCIENCE Eds. Coligan et al, (John Wiley & Sons, Inc. NY USA 1995-2008), in particular Chapters 1, 5 and 6. Typically, recombinant protein preparation includes expression of a nucleic acid encoding the protein in a suitable host cell.

The present disclosure further contemplates humanized or at least partly humanized (also referred to as “humaneered”) versions of the single domain antibodies provided herein. Accordingly, in some examples, the single domain antibody provided herein is a humanized or substantially humanized single domain antibody.

By “humanized” is meant the amino acid sequence of the single domain antibody is mutated or modified so that immunogenicity upon administration in human patients is reduced, minor or non-existent (e.g., a single domain antibody that originated from a species other than human that has had immunogenic or potentially immunogenic amino acid residues replaced with amino acids that are less immunogenic or not immunogenic in the context of a single domain antibody administered to a human subject). Accordingly, humanized single domain antibodies should be substantially non-immunogenic in humans, but retain the affinity and activity of the wild-type, unmodified or camelid single domain antibody (e.g., the single domain antibodies of SEQ ID NOs: 1-4).

Any method known in the art for creating humanized antibodies are envisaged herein, including but not limited to the humanizing technology of KaloBios Pharmaceuticals and that described in Vincke et al. (JBC 2008). By way of example, humanising a single domain antibody generally comprises a step of replacing one or more of the camelid- or alpaca-derived amino acid residues with their human counterpart as found in a corresponding human consensus sequence, without that single domain antibody losing its typical character or biological function. Suitably, humanization does not significantly affect the antigen binding capacity of the resulting humanized single domain antibody. Notwithstanding this, humanizing modifications or mutations may be made in one or more CDRs and/or FRs of the single domain antibodies. In particular examples, the humanized single domain antibody may contain one or more fully human FR sequences, as are known in the art.

### Antigen Binding Molecules

The present disclosure further provides an antigen binding molecule that includes the single domain antibody described herein.

An antigen binding molecule is one which includes a single domain antibody disclosed herein (e.g., any of SEQ ID NOs: 1-4 or a variant thereof) and one or more functional moieties, such as one or more further antigen binding moieties, domains or units in addition to the single domain antibody.

As such, in a particular form, the antigen binding molecule comprises a single domain antibody disclosed herein and one or more further antigen binding moieties, domains or units.

Exemplary antigen binding moieties, domains or units include single domain antibodies (inclusive of those described herein and further single domain antibodies, such as those directed against an antigen of a norovirus as are known in the art), monoclonal antibodies (mAbs) (e.g., 8C7, 5B18, 1B4, 1F6, N2C3), antibody mimetic proteins, aptamers and antibody fragments, such as Fc, Fab or F (ab) 2 fragments and/or may comprise single chain Fv antibodies (scFvs). Such scFvs may be prepared, for example, in accordance with the methods described respectively in U.S. Pat. No. 5,091,513, European Patent No 239,400 or the article by Winter & Milstein, 1991, Nature 349:293. Antigen binding moieties, domains or units may also include multivalent recombinant antibody fragments, such as diabodies, triabodies and/or tetrabodies, comprising a plurality of scFvs, as well as dimerisation-activated demibodies (e.g. WO/2007/062466). By way of example, such antibodies may be prepared in accordance with the methods described in Holliger et al., 1993 Proc Natl Acad Sci USA 90 6444; or in Kipriyanov, 2009 Methods Mol Biol 562 177. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbour, Cold Spring Harbour Laboratory, 1988.

Suitably, the antigen binding molecule is a monospecific antigen binding molecule. A monospecific antigen binding molecule binds a single antigen (e.g., a VP1 protein of a norovirus). In some examples, the monospecific antigen binding molecule includes one or more of the single domain antibodies provided herein. More particularly, the monospecific antigen binding molecule may comprise two or more (e.g., 2, 3, 4, 5, 6 etc) of the single domain antibodies provided herein. In this regard, the antigen binding molecule may comprise, for example, a dimer, trimer, quadramer, tetramer, hexamer etc of a single domain antibody described herein. For such examples, each of the single domain antibodies of the antigen binding molecule can comprise the same or substantially the same amino acid sequence connected by a linker, such as a polypeptide linker as are known in the art.

Suitably, the antigen binding molecule is a multispecific antigen binding molecule, such as one that comprises one or more of the single domain antibodies provided herein and one or more (e.g., 1, 2, 3, 4, 5, 6 etc) further binding moieties, domains or units (e.g., one or more further single domain antibodies) that bind to one or more respective epitopes other than to which the single domain antibody disclosed herein is raised against or binds (e.g., an epitope other than that of a P2 subdomain of a VP1 protein of a norovirus, such as an antigen of a norovirus other than an VP1 protein thereof or an epitope within a P1 subdomain of the VP1 protein). In particular examples, the antigen binding molecule comprises a single domain antibody provided herein that is directed to a P2 subdomain of a VP1 protein and a further antigen binding moiety, domain or unit that is directed to a P1 subdomain of the VP1 protein. To this end, the further antigen binding moiety, domain or unit that is directed to the P1 subdomain of the VP1 protein may be any as is known in the art, inclusive of functional fragments or derivatives thereof.

A number of anti-norovirus antibodies and nanobodies binding to a norovirus VP1 protein have been published, such as MAM4-1418, binding to residues 418 to 426 and 526 to 534 thereof (Shiota T et al, J Virol. 2007 November; 81(22): 12298-306); NV23, NS22, and F120, binding to residues 453-472 thereof, NS14, binding to residues 473-494 thereof, 8C7, binding to residues 451-489 thereof (Crawford S E et al, Clin Vaccine Immunol. 2015 February; 22(2): 168-77; Kou B et al., Clin Vaccine Immunol. 2015 February; 22(2): 160-7), 5B18, binding to residues 433, 496, 533-535 thereof (Hansman G S et al. J Virol. 2012 April; 86(7): 3635-46), NV3901 and NV3912, binding to residues 454-520 thereof (Parker T D et al, J Virol. 2005 June; 79(12): 7402-9), 1B4 and 1F6, binding to residues 45-55 thereof (Yoda T et al, J Clin Microbiol. 2003 June; 41(6): 2367-71), N2C3, binding to residues 55-60 thereof (Li X et al, Virus Res. 2009 March; 140(1-2): 188-93, Li X et al, Virus Res. 2010 August; 151(2): 142-7), an antibody binding to residues 52-56 thereof (Gabriel I. Parra et al. PLOS One. 2013; 8(6): e67592), Nano-85, binding to residues 517-526 thereof (WO 2016/059113; Koromyslova and Hansman, J Virol, 2015, see reference 35) and Nano-26 binding to residues 269 to 276 thereof (WO2019057755; Koromyslova and Hansman, PLOS Pathog, 2017, see reference 34), which are incorporated by reference herein.

Suitably, the further antigen binding moiety, domain or unit is that of nano-85 described in WO2016059113 or a functional fragment, variant or derivative thereof. As such, in various examples, the antigen binding molecule comprises a further single domain antibody that is directed to (e.g., binds to or is raised against) an amino acid sequence of W-V-N-X¹-F-Y-X² (SEQ ID NO: 25), wherein X¹ represents any amino acid, preferably Q or P, and X² represents any amino acid, preferably T or S, of a VP1 protein of a norovirus. For such examples, the further single-domain antibody suitably comprises the complementarity determining regions (CDRs) (i) CDR1: GSIFSIYA (SEQ ID NO: 26), GSIFSIYL (SEQ ID NO: 27) or a variant thereof, (ii) CDR2: ISSGGGTN (SEQ ID NO: 28) or a variant thereof, and (iii) CDR3: KREDYSAYAPPSGS (SEQ ID NO: 29), KREDFSAYAPPSGS (SEQ ID NO: 30) or a variant thereof. Even more particularly, the further single-domain antibody suitably comprises, consists of or consists essentially of the amino acid sequence of SEQ ID NO: 31, or a fragment, variant or derivative thereof.

Suitably, the further antigen binding moiety, domain or unit is that of nano-26 described in WO2019057755 or a functional fragment, variant or derivative thereof. As such, according to other examples, the antigen binding molecule comprises a further single domain antibody that is directed to a first epitope having an amino acid sequence of a-x-a-h-x-h-x-o (SEQ ID NO:32), with “x” being any amino acid; “a” being glutamic acid or aspartic acid; “h” being glycine, alanine, valine, leucine or isoleucine, and “o” being serine or threonine, of a VP1 protein of a norovirus. More particularly, said first epitope comprises the motif D-x-E-L-x-G-x-T (SEQ ID NO:33) with “x” being any amino acid. The further single domain antibody may further bind a second epitope on a VP1 protein of a norovirus, wherein said second epitope comprises the motif N or Q-D or E, preferably Q-E, more particularly N or Q-D or E-(x)₁₅-P (SEQ ID NO:34), even more particularly Y-Q-E-S-x-P-(x)₁₂-P (SEQ ID NO:35), with “x” being any amino acid. For such examples, the further single-domain antibody suitably comprises the complementarity determining regions (CDRs) (i) CDR1: RIIFFMYD (SEQ ID NO: 36) or a variant thereof, (ii) CDR2: QINSDVST (SEQ ID NO: 37) or a variant thereof, and (iii) CDR3: YCNVRRASA (SEQ ID NO: 38) or a variant thereof. Even more particularly, the further single-domain antibody suitably comprises, consists of or consists essentially of the amino acid sequence of SEQ ID NO: 39, or a fragment, variant or derivative thereof.

In certain examples, the antigen binding molecule is or comprises a monovalent antigen binding molecule. A monovalent antigen binding molecule is one that comprises a single antigen-binding moiety, domain or unit (e.g., a single nanobody or VHH chain), such as those provided herein.

In other examples, the antigen binding molecule is or comprises a multivalent antigen binding molecule, such as a bivalent antigen binding molecule. A multivalent antigen binding molecule comprises two or more antigen binding moieties, domains or units (e.g., a single domain antibody dimer, trimer, quadramer, pentamer, hexamer etc). In some examples, the antigen binding molecule is or comprises a bivalent antigen binding molecule. A bivalent antigen binding molecule comprises two antigen-binding moieties, domains or units (i.e., 2 single domain antibodies, such as one or two of those provided herein).

Accordingly, if one single domain antibody described herein is linked to another single domain antibody, such as one of the present disclosure, the single domain antibody has a bivalent format, and if, for example, three single domain antibodies, such as those of the present disclosure, are linked together, the antigen binding molecule has a trivalent format. Such formats can be desirable to enhance or otherwise modify the effectiveness of the antigen binding molecules or single domain antibodies of the present disclosure. To generate multivalent antigen binding molecules as described herein, respective or adjacent antigen-binding moieties, domains or units may be connected by a linker, such as a polypeptide linker.

According to particular examples, the antigen binding molecule provided herein is multiparatopic. A multiparatopic antigen binding molecule binds the same antigen, but the respective antigen binding moieties, domains or units thereof bind to more than one epitope (i.e., different epitopes) in said antigen (e.g., a P1 subdomain and a P2 subdomain of a VP1 protein). In certain examples, the antigen binding molecule is a biparatopic antigen binding molecule. A biparatopic antigen binding molecule is monospecific, but the respective binding moieties, domains or units thereof bind to two different epitopes of the same antigen (e.g., a P domain of a VP1 protein). In such examples, the antigen binding molecule can comprise a single domain antibody provided herein and a further antigen binding moiety, domain or unit, such as a further single domain antibody, that binds to the P domain of a VP1 protein, but at a different or overlapping epitope to that of said single domain antibody.

According to various examples, the antigen binding molecule comprises a single domain antibody disclosed herein and optionally one or more binding moieties, domains or units conjugated or otherwise linked to one or more functional moieties. To this end, the antigen binding molecule may be described as an immunoconjugate. As used herein, the term “immunoconjugate” refers to a polypeptide molecule that includes at least one functional moiety and an antigen binding moiety, such as a single domain antibody disclosed herein.

Exemplary functional moieties include a detectable marker or label, a therapeutic agent, a half-life extender and a nanocarrier, inclusive of combinations thereof.

In certain examples, the functional moiety is or comprises a half-life extender that may serve to prolong the half-life of the antigen binding molecule or single domain antibody in vivo following administration to a subject. Such half-life extenders may comprise, for example, an antibody, or part thereof, or a protein, or part thereof, that binds or is derived from a serum albumin (e.g., a serum albumin protein, an albumin binding domain). In particular examples, the half-life extender is or comprises an Fc domain or region. Other half-life extenders that may be utilised for the present disclosure include polymers, such as polyethylene glycol (PEG) and starch. Half-life may be increased by at least 1.5 times, more particularly at least 2 times, even more particularly at least 5 times, yet even more particularly at least 10 times or still even more particularly at least 20 times, greater than the half-life of the corresponding single domain antibodies or antigen binding molecules of the present disclosure that do not include such a half-life extender. For example, the half-life may be increased by more than 1 hour, more particularly more than 2 hours, even more particularly more than 6 hours, yet even more particularly more than 12 hours, or still even more particularly more than 24, 48 or 72 hours, compared to the single domain antibodies or antigen binding molecules of the present disclosure that do not include such a half-life extender.

According to other examples, the single domain antibodies or antigen binding molecules of the present disclosure are labelled with a detectable or functional marker or label. A label can be any molecule that produces or can be induced to produce a signal, including but not limited to fluorophores, fluorescent labels, radiolabels, enzymes, chemiluminescent labels, a nuclear magnetic resonance active label or photosensitizers, as described in more detail herein. Thus, binding of the labelled single domain antibody or antigen binding molecule to a P domain of a norovirus may be detected and/or measured by detecting fluorescence, luminescence, radioactivity, enzyme activity or light absorbance thereof.

For some examples, the single domain antibodies or antigen binding molecules of the present disclosure are coupled to a therapeutic agent or moiety (e.g., a small molecule, a protein, a nucleic acid, such as an siRNA), such as a drug, an enzyme, a cytokine (e.g., IL-2, IL-12, and TNF), a radionuclide or a toxin (e.g., Enterobacter cloacae β-Lactamase, Pseudomonas Exotoxin A, TRAIL and granzyme B). In particular examples, the therapeutic agent is an antiviral agent, as are known in the art. Exemplary antiviral agents include dasabuvir, nitazoxanide, remdesivir and nelfinavir.

In some examples, the single domain antibodies or antigen binding molecules of the present disclosure are linked with, coupled to or otherwise associated with a nanocarrier, such as a liposome, a micelle, a lipid nanoparticle, albumin-based nanoparticles and polymer-based nanoparticles. Because of the binding specificity of single domain antibodies, they may be broadly used in such drug delivery platforms to deliver their cargo (e.g., a therapeutic payload) to its specific location (e.g., a viral particle).

It is envisaged that all combinations of the antigen binding molecule, in particular those combinations specifically listed herein, can be used in any therapeutic, diagnostic, or prognostic method or use, such as those hereinafter described.

### Encoding Nucleic Acids

The present disclosure also provides an isolated nucleic acid encoding the single domain antibody or the antigen binding molecule described herein.

The term “nucleic acid” as used herein designates single- or double-stranded DNA and RNA. DNA includes genomic DNA and cDNA. RNA includes mRNA, RNA, RNAi, siRNA, CRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as modified purines (for example inosine, methylinosine and methyladenosine) and modified pyrimidines (for example thiouridine and methylcytosine).

As used herein, a “polynucleotide” is a nucleic acid having eighty (80) or more contiguous nucleotides, while an “oligonucleotide” has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

Suitably, the isolated nucleic acid encodes a single domain antibody, such as a single domain antibody comprising:
(a) a CDR1 that comprises an amino acid sequence of ASGRFFSSYA (SEQ ID NO: 5), ASGRTESSY (SEQ ID NO: 8), RTDSEST (SEQ ID NO: 11), SGTIFSIDA (SEQ ID NO: 14) or a variant thereof;
(b) a CDR2 that comprises an amino acid sequence of ISWSGGST (SEQ ID NO: 6), TGSGD (SEQ ID NO: 9), WRYA (SEQ ID NO: 12), QAPGKQRE (SEQ ID NO: 15) or a variant thereof; and
(c) a CDR3 that comprises an amino acid sequence of AREGAYYPDSYYRTVRYD (SEQ ID NO: 7), YRTGGPPQW (SEQ ID NO: 10), RYIYGSLSDSGSYDN (SEQ ID NO: 13), AKPPTYYSLEPWGKGT (SEQ ID NO: 16) or a variant thereof.

More particularly, the isolated nucleic acid encodes a single domain antibody comprising:
(i) the CDR1 that comprises the amino acid sequence of ASGRFFSSYA (SEQ ID NO: 5) or a variant thereof, the CDR2 that comprises the amino acid sequence of ISWSGGST (SEQ ID NO: 6) or a variant thereof and the CDR3 that comprises the amino acid sequence of AREGAYYPDSYYRTVRYD (SEQ ID NO: 7) or a variant thereof;
(ii) the CDR1 that comprises the amino acid sequence of ASGRTESSY (SEQ ID NO: 8) or a variant thereof, the CDR2 that comprises the amino acid sequence of TGSGD (SEQ ID NO: 9) or a variant thereof and the CDR3 that comprises the amino acid sequence of YRTGGPPQW (SEQ ID NO: 10) or a variant thereof;
(iii) the CDR1 that comprises the amino acid sequence of RTDSEST (SEQ ID NO: 11) or a variant thereof, the CDR2 that comprises the amino acid sequence of WRYA (SEQ ID NO: 12) or a variant thereof and the CDR3 that comprises the amino acid sequence of RYIYGSLSDSGSYDN (SEQ ID NO: 13) or a variant thereof; or
(iv) the CDR1 that comprises the amino acid sequence of SGTIFSIDA (SEQ ID NO: 14) or a variant thereof, the CDR2 that comprises the amino acid sequence of QAPGKQRE (SEQ ID NO: 15) or a variant thereof and the CDR3 that comprises the amino acid sequence of AKPPTYYSLEPWGKGT (SEQ ID NO: 16) or a variant thereof.

In some examples, the isolated nucleic acid encodes the single domain antibody comprising, consisting essentially of or consisting of the amino acid sequence set forth in any of SEQ ID NOs: 1 to 4, or a fragment, derivative or variant thereof.

Suitably, the isolated nucleic acid encoding the single domain antibody comprises, consists essentially of or consists of the nucleotide sequence set forth in any of SEQ ID NOs: 17 to 20, or a fragment, derivative or variant thereof.

Also contemplated are fragments and variants of the isolated nucleic acid. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence encoding the single domain antibody or the antigen binding molecule of the present disclosure (e.g., SEQ ID NOs: 17 to 20).

Fragments of the isolated nucleic acid may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence encoding the single domain antibody or antigen binding molecule of the present disclosure, such that they encode at least a portion of the single domain antibody or antigen binding molecule. In general, fragments may comprise, consist essentially of or consist of up to 150, 165, 180, 195, 210, 225, 240, 255, 270, 285, 300, 315, 330, 345, 360, 375, 390, 405 or 411, contiguous nucleic acids that encode a portion of a single domain antibody or an antigen binding molecule described herein (e.g., SEQ ID NOS: 1-4).

The present disclosure also provides nucleic acids that have been modified such as by taking advantage of codon sequence redundancy. In a more particular example, codon usage may be modified to optimize expression of a nucleic acid in a particular organism or cell type.

The isolated nucleic acids disclosed herein can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008). Nucleic acids of the present disclosure may be isolated, detected and/or subjected to recombinant DNA technology using nucleic acid sequence amplification techniques. Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-β replicase amplification, recombinase polymerase amplification (RPA) and helicase-dependent amplification, although without limitation thereto.

### Genetic Constructs

The present disclosure also provides a genetic construct comprising the isolated nucleic acid hereinbefore described. The genetic construct may be a vector.

In particular examples, the genetic construct comprises the isolated nucleic acid operably linked or connected to one or more other genetic components. A genetic construct may be suitable for therapeutic delivery of the isolated nucleic acid or for recombinant production of the single domain antibody or the antigen binding molecule of the disclosure in a host cell.

Broadly, the genetic construct can be in the form of, or comprises genetic components of, a plasmid, bacteriophage, a cosmid, a yeast or bacterial artificial chromosome as are well understood in the art. Genetic constructs may be suitable for maintenance and propagation of the isolated nucleic acid in bacteria or other host cells, for manipulation by recombinant DNA technology and/or expression of the nucleic acid or an encoded protein of the present disclosure.

For the purposes of host cell expression, the genetic construct is an expression construct. Suitably, the expression construct comprises the nucleic acid of the present disclosure operably linked to one or more additional sequences in an expression vector. An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the present disclosure preferably to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, polyadenylation sequences, transcriptional start and termination sequences, translational start and termination sequences, and enhancer or activator sequences. Constitutive, repressible or inducible promoters as known in the art are contemplated by the present disclosure.

The expression construct may also include an additional nucleotide sequence encoding a fusion partner (typically provided by the expression vector) so that the recombinant protein is expressed as a fusion protein.

The expression construct may also include an additional nucleotide sequence encoding a selection marker such as amp^{R}, neo^{R} or kan^{R}, although without limitation thereto.

### Host Cells

The present disclosure also provides a host cell transformed with a nucleic acid molecule or a genetic construct described herein. The host cell may be an isolated host cell or a cell in vitro.

Suitable host cells for expression may be prokaryotic or eukaryotic. For example, suitable host cells may include but are not limited to mammalian cells (e.g. HeLa, Cos, NIH-3T3, HEK293T, Jurkat, CHO cells), yeast cells (e.g. Saccharomyces cerevisiae), insect cells (e.g., Sf9, Trichoplusia ni) utilized with or without a baculovirus expression system, plant cells (e.g. Chlamydomonas reinhardtii, Phaeodactylum tricornutum) or bacterial cells, such as E. coli. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2015), in particular Chapters 9 and 16.

### Methods of Production

Related aspects of the present disclosure provide a method of producing the single domain antibody or the antigen binding molecule described herein, including the steps of; (i) culturing the host cell disclosed herein; and (ii) isolating the single domain antibody or the antigen binding molecule from said host cell cultured in step (i).

In this regard, the recombinant protein may be conveniently prepared by a person skilled in the art using standard protocols, such as those hereinbefore provided.

The present disclosure further provides a single domain antibody or an antigen binding molecule produced by the method described herein.

### Pharmaceutical Compositions

Further aspects of the present disclosure provide a composition comprising the single domain antibody and/or the antigen binding molecule described herein, and one or more pharmaceutically acceptable carriers, diluents or excipients.

By “pharmaceutically-acceptable carrier, diluent or excipient” is meant a solid or liquid filler, diluent or encapsulating substance that may be safely used in systemic administration. Depending upon the particular route of administration, a variety of carriers well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water,

A useful reference describing pharmaceutically acceptable carriers, diluents and excipients is Remington's Pharmaceutical Sciences (Mack Publishing Co. NJ. USA, 1991), which is incorporated herein by reference.

In some examples, the present composition is in the form of a diagnostic composition.

In other examples, the present composition is in the form of a therapeutic composition.

A therapeutically effective amount of a composition comprising a single domain antibody and/or an antigen binding molecule may be administered in a single dose, or in several doses, for example daily, during a course of treatment. However, the frequency of administration is dependent on the preparation applied, the subject being treated, the severity of the viral infection, and the manner of administration of the therapy or composition.

Any safe route of administration may be employed for administering the single domain antibodies and antigen binding molecules described herein. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be achieved by coating the same, for example, with hydrophobic polymers including acrylic resins, waxes, higher aliphatic alcohols, polylactic and polyglycolic acids arid certain cellulose derivatives such as hydroxypropylmethyl cellulose, in addition, the controlled release may be achieved by using other polymer matrices, liposomes and/or microspheres. In particular examples, the composition is capable of being or configured or adapted to be administered orally to a subject in need thereof. For such examples, the composition is suitably enterically coated.

Compositions of the present disclosure suitable for oral or parenteral administration may be presented as discrete units such as capsules, sachets or tablets each containing a pre-determined amount of one or more therapeutic agents of the disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more therapeutic agents as described above with the carrier which constitutes one or more necessary ingredients. In general, the compositions are prepared by uniformly and intimately admixing the therapeutic agents of the disclosure with liquid carriers or finely divided solid carriers or both, and then, if necessary, shaping the product into the desired presentation.

The above compositions may be administered in a manner compatible with the dosage formulation, and in such an amount as is effective to prophylactically and/or therapeutically treat noroviral infections, and/or diseases, disorders or conditions associated therewith and/or alleviate symptoms associated therewith. The dose administered to a patient, in the context of the present disclosure, should be sufficient to achieve a beneficial response in a patient over time such as a reduction in a level of viral shedding in their bodily fluids (e.g., blood, urine, saliva). The quantity of the therapeutic agent(s) to be administered may depend on the subject to be treated inclusive of the age, sex, weight and general health condition thereof. In this regard, precise amounts of the therapeutic agent(s) required to be administered will depend on the judgement of the clinician. The total dose required for each treatment may be administered by multiple doses or in a single dose. In any event, suitable dosages of the therapeutic agents described herein may be readily determined by those skilled in the art. Such dosages may be in the order of nanograms to milligrams of the therapeutic agents of the disclosure.

Suitably, the composition further comprises one or more further or additional therapeutic agents, such as an anti-inflammatory agent (e.g., NSAIDs, corticosteroids) and/or an antiviral agent (e.g., nano-26, nano-85, dasabuvir, nitazoxanide, remdesivir and nelfinavir). In particular examples, the composition further includes a further single domain antibody that binds to the P domain, such as the P1 subdomain, of a VP1 protein of a norovirus. In various examples, the composition further comprises a nano-26 single domain antibody or a functional variant, fragment or derivative thereof. In some examples, the composition further comprises a nano-85 single domain antibody or a functional variant, fragment or derivative thereof.

### Methods for Preventing and Treating Noroviral Infections

In view of the foregoing, the single domain antibodies, antigen binding molecules, nucleic acids, genetic constructs, host cells and compositions described herein can be utilised to prevent, ameliorate and/or treat noroviral infections, such as those caused by a GII genogroup, and more particularly a GII.4 genotype or a GII.17 genotype, and/or diseases, disorders or conditions associated therewith.

In one broad form, the present disclosure provides method of inhibiting or preventing binding of a norovirus, such as a viral particle thereof, to a histo-blood group antigen (HBGA) and/or a bile acid in a subject, said method including the step of administering to the subject a therapeutically effective amount of the single domain antibody, the antigen binding molecule, the isolated nucleic acid, the genetic construct, the host cell or the composition disclosed herein to thereby inhibit or prevent binding of the norovirus to the HBGA and/or the bile acid in the subject.

In a related form, the present disclosure relates to the use of the single domain antibody, the antigen binding molecule, the isolated nucleic acid, the genetic construct, the host cell or the composition disclosed herein in the manufacture of a medicament for inhibiting or preventing binding of a norovirus to a HBGA and/or a bile acid in a subject.

In yet a further related form, the present disclosure relates to the single domain antibody, the antigen binding molecule, the isolated nucleic acid, the genetic construct, the host cell or the composition disclosed herein for use in inhibiting or preventing binding of a norovirus to a HBGA and/or a bile acid in a subject.

In another broad form, the present disclosure relates to the use of a single domain antibody, an antigen binding molecule, a composition, an isolated nucleic acid, a genetic construct, a host cell described herein for therapy.

Accordingly, there is provided herein a method of treating and/or preventing a norovirus infection, and/or a disease, disorder or condition associated therewith in a subject, said method including the step of administering to the subject a therapeutically effective amount of the single domain antibody, the antigen binding molecule, the isolated nucleic acid, the genetic construct, the host cell or the composition disclosed herein to thereby treat or prevent the norovirus infection and/or disease, disorder or condition associated therewith in the subject.

In another form, there is provided the use of the single domain antibody, the antigen binding molecule, the isolated nucleic acid, the genetic construct, the host cell or the composition disclosed herein in the manufacture of a medicament for the treatment and/or prevention of a norovirus infection and/or a disease, disorder or condition associated therewith in a subject.

In yet a further related form, the present disclosure relates to the single domain antibody, the antigen binding molecule, the isolated nucleic acid, the genetic construct, the host cell or the composition disclosed herein for use in the treatment and/or prevention of a norovirus infection and/or a disease, disorder or condition associated therewith in a subject.

In relation to the above aspects of the present disclosure, the norovirus infection can be the result of any norovirus genogroup, genotype, strain or variant as are known in the art and provided herein. In particular examples, the norovirus infection can be a GII.4 norovirus infection and/or a disease, disorder or condition associated therewith. In other examples, the norovirus infection can be a GII. 17 norovirus infection and/or a disease, disorder or condition associated therewith.

As used herein, “treating” (or “treat” or “treatment”) refers to a therapeutic intervention that ameliorates a sign or symptom of a disease, disorder or condition characterized by a norovirus infection, after it has begun to develop. The term “ameliorating”, with, reference to such diseases, disorders or conditions, refers to any observable beneficial effect of the treatment. Treatment need not be absolute to be beneficial to the subject. The beneficial effect can be determined using any methods or standards known to the ordinarily skilled artisan.

As used herein, “preventing” (or “prevent” or “prevention”) refers to a course of action (such as administering a therapeutically effective amount of the single domain antibody) initiated prior to the onset of a symptom, aspect, or characteristic of a norovirus infection so as to prevent or reduce the symptom, aspect, or characteristic. It is to be understood that such preventing need not be absolute to be beneficial to a subject.

Prophylactic administration of the single domain antibodies, antigen binding molecules, isolated nucleic acids, genetic constructs, host cells and compositions described herein is also envisaged for the present disclosure, particularly in respect of patients who have had prior exposure or contact with an animal or human known or suspected to have been infected with a norovirus. A “prophylactic” treatment, such as passive immunisation, is a treatment administered to a subject who does not exhibit signs of the disease, disorder or condition or exhibits only early signs for the purpose of decreasing the risk of developing a symptom, aspect, or characteristic thereof.

By “administration” is meant the introduction of a composition (e.g., a composition comprising a single domain antibody or an encoding nucleic acid) into a subject by a chosen route. In some examples, the therapeutically effective amount of the composition is administered subcutaneously. In other examples, the therapeutically effective amount of the composition is administered intramuscularly. In various examples, the therapeutically effective amount of the composition is administered intravenously. In some examples, the therapeutically effective amount of the composition is administered by inhalation. In particular examples, the therapeutically effective amount of the composition is administered by lumbar puncture (spinal tap). In particular examples, the therapeutically effective amount of the composition is administered orally.

The term “therapeutically effective amount” describes a quantity of a specified agent sufficient to achieve a desired effect in a subject being treated with that agent. For example, this can be the amount of a composition comprising the single domain antibody, antigen binding molecule or isolated nucleic acid necessary to reduce, alleviate and/or prevent a norovirus infection. In some examples, a “therapeutically effective amount” is sufficient to reduce or eliminate a symptom of the norovirus infection. In other examples, a “therapeutically effective amount” is an amount sufficient, to achieve a desired biological effect, for example, an amount that is effective to decrease a symptom associated with a norovirus infection.

Ideally, a therapeutically effective amount of an agent is an amount sufficient to induce the desired result without causing a substantial cytotoxic effect in the subject. The therapeutically effective amount of an agent, such as a single domain antibody, useful for reducing, alleviating and/or preventing a norovirus infection will be dependent on the subject being treated, the type and severity of any associated disease, disorder and/or condition, and the manner of administration of the therapeutic composition.

With respect to the aspects described herein, the term “subject” includes, but is not limited to, mammals, inclusive of humans, performance animals (such as horses, camels, greyhounds), livestock (such as pigs, cattle, sheep, horses) and companion animals (such as cats and dogs). In certain examples, the subject is a human. In other examples, the subject is a dog. In various examples, the subject is a bovine. In other examples, the subject is a pig.

As used herein, the terms “a disease, disorder or condition associated with norovirus” refer to diseases, disorders or conditions caused, directly or indirectly, by infection with a norovirus. This virus can be amplified and cause severe disease in domestic animals, such as pigs and cattle. norovirus may then be subsequently transmitted to humans (i.e., a zoonotic disease), where the infection is highly contagious and typically manifested as a severe gastrointestinal illness that may include nausea, vomiting, stomach pain or cramps, watery or loose diarrhoea, feeling ill, low-grade fever and myalgia.

### Methods for Diagnosing Noroviral Infections

The single domain antibodies and antigen binding molecules disclosed herein may be used to detect viral proteins or antigens, such as a VP1 or capsid protein of a norovirus, in vitro or in vivo. Such in vitro testing may involve obtaining a biological sample, such as faeces, saliva, vomit, mucous, blood, plasma or serum, from the subject. The detection of viral protein or elevated levels of viral protein in the biological sample from a subject may be indicative of a norovirus infection in said subject.

Accordingly, in one broad form, the present disclosure provides a method of diagnosing or monitoring a norovirus infection, and/or a disease, disorder or condition associated therewith, in a subject, said method including the step of contacting the subject and/or a biological sample from the subject with a single domain antibody, an antigen binding molecule or a composition described herein.

The present methods may include the step of determining a presence or absence of a norovirus infection in said subject, wherein the presence of a viral protein, such as an VP1 protein, of a norovirus, indicates a current or previous norovirus infection. As such, the subject and/or a biological sample therefrom may be contacted with the single domain antibody, the antigen binding molecule or the composition for a time and under conditions sufficient to detect antigen-specific binding thereof. Suitably, the method of this aspect is for determining a relative or absolute level of the viral protein in the biological sample.

The present method may further include the earlier step of collecting the biological sample from the subject. Such a sample may be obtained by freshly collecting a sample, or may be obtained from a previously collected and stored sample. By way of example, a sample may be obtained from a previously collected and stored (e.g., refrigerated or frozen) stool or vomit sample. Suitably, a sample is obtained by freshly collecting a sample from the subject. Alternatively, a sample can be obtained from a previously collected and stored sample from the subject. Once collected the sample may be processed in a way, such as purifying, concentrating or solubilising, to make it more suitable for the subsequent contacting and/or detection steps (e.g., concentration, isolation or purification of one or more virus particles or proteins within the biological sample). Such assays, may include immunoassays, such as western blot and ELISA. It should be understood, however, that this disclosure is not limited by reference to the specific methods of detection or immunoassays disclosed herein.

In some examples, the methods described herein may be performed in a lateral flow assay (LFA) format. LFAs, also known as “immunochromatographic strip tests”, have been a popular platform for rapid immunoassays since their introduction in the mid-1980s. LFAs are particularly suitable where a rapid test is required or where specialized laboratory equipment is not available. In hospitals, clinics, physician offices, and clinical laboratories, LF-based tests are used for the qualitative and quantitative detection of the presence of a specific analyte in a liquid sample. LFAs operate on the same principles as ELISA. In essence, these tests run a liquid sample along the surface of a membrane or filter paper with reactive molecules that show a visual positive or negative result depending on the presence of a particular analyte. A LFA device, is a device configured to receive a sample at a sample region and to provide for the sample to move laterally, via, e.g., wicking, by capillary action from the sample region to a detection region.

The methods of the present disclosure can be performed on various biological samples. As used herein, the term “biological sample” is suitably a sample obtained from a subject. For example, the biological sample can be a bodily fluid of the subject. In certain examples, the biological sample is selected from a group consisting of blood, serum, plasma, urine, saliva, faeces, tears, vomit, broncho-alveolar lavage fluid (BALF), cerebrospinal fluid (CSF) and seminal fluid. In certain examples, the biological sample is a faecal or stool sample. In other examples, the biological sample is a vomit sample. In some examples, the biological sample is a mucous sample. For particular examples, the biological sample is a saliva sample.

In another form, the present disclosure provides a method of isolating or purifying a norovirus from a sample, such as a biological sample, said method including the steps of:
(a) contacting the sample with the single domain antibody or antigen binding molecule described herein; and
(b) isolating a norovirus: single domain antibody complex or a norovirus:
antigen binding molecule complex from the sample,
   to thereby isolate or purify a norovirus from the sample.

Methods for isolating or purifying a norovirus or an immune complex comprising same from a sample will be apparent to the skilled person and/or are described herein.

In this context, the term “isolating” suitably refers to at least partly purifying, concentrating or removing the norovirus from the sample. Affinity based separation methods may be used. A tag or label on a single domain antibody or antigen binding molecule described herein may also be used so that the norovirus can be filtered or sorted from a sample, such as by a fluorescence- or affinity chromatography-based sorting system.

Suitably, the single domain antibody or antigen binding molecules for use in the present methods are labelled, such as by way of conjugation to a detectable or functional label or marker. Various methods of labelling proteins are known in the art and may be used. Examples of labels for polypeptides include, but are not limited to, radioisotopes or radionucleotides (such as ³⁵S, ¹¹C, ¹³N, ¹⁵O, ¹⁸F, ¹⁹F, ⁹⁹TC, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents (such as gadolinium chelates). In some examples, labels are conjugated or operably linked to the single domain antibody or antigen binding molecules by one or more linkers of various lengths to reduce potential steric hindrance for the binding of the single domain antibody or antigen binding molecules disclosed herein to a viral protein.

Suitably, the present methods further include the step of detecting and/or measuring a level of antigen binding to the single domain antibody or the antigen binding molecule. Any method or assay known in the field can be employed in the diagnostic and prognostic methods of the invention, e.g. ligand binding assays, immunoassays, competition binding assays, etc., for detecting and/or measuring antigen determining the presence of a norovirus or a viral protein or antigen derived therefrom.

Suitably, the diagnostic methods described herein may include the step of administering a treatment to the subject. By way of example, this can include administering to the subject a therapeutically effective amount of a treatment, such as those therapeutic agents, inclusive of the single domain antibodies, described herein, when one or more noroviral proteins or antigens (e.g., VP1) are detected in the subject or a biological sample obtained therefrom (e.g., method is diagnostic or indicative of the subject having a noroviral infection). More particularly, this may include administering to the subject a therapeutically effective amount of an anti-viral agent, such as a single domain antibody described herein, when the method is diagnostic or indicative of the subject having a noroviral infection.

It is envisaged that the single domain antibody or antigen binding molecules disclosed herein can be used in vitro and in vivo to monitor the course of a norovirus infection, such as during therapy thereof. Thus, for example, by measuring the increase or decrease in a level of a viral protein/antigen or the number of cells infected with a norovirus or changes in the subject or in a biological sample therefrom, it would be possible to determine whether a particular therapeutic regimen aimed at treating or ameliorating a norovirus infection, and/or a disease, disorder or condition associated therewith, is effective.

### Nucleic Acid Delivery

The present disclosure also provides certain aspects relating to the administration of one or more nucleic acids encoding the single domain antibodies or antigen binding molecules described herein (e.g., a single domain antibody comprising, consisting essentially of or consisting of an amino acid sequence set forth in any of SEQ ID NOs: 1-4), or fragments, variants or derivatives thereof, for:
(i) passively immunizing a subject against a norovirus infection; and/or
(ii) treating, ameliorating or preventing a norovirus infection in a subject.

Accordingly, in one particular form, there is provided herein a method of treating and/or preventing a norovirus infection, and/or a disease, disorder or condition associated therewith in a subject, said method including the step of administering to the subject a therapeutically effective amount of an isolated nucleic acid encoding a single domain antibody or an antigen binding molecule provided herein.

In some examples, the isolated nucleic acid encoding the single domain antibody or the antigen binding molecule is in the form of a genetic construct suitable for administration to a mammal such as a human. More particularly, the genetic construct may be suitable for DNA delivery of the single domain antibodies or antigen binding molecules to a mammal such as a human. A useful reference describing DNA delivery of peptides is DNA Vaccines, Methods and Protocols, Second Edition (Volume 127 of Methods in Molecular Medicine series, Humana Press, 2006).

According to certain examples, the isolated nucleic acid encoding the single domain antibody or antigen binding molecule is in the form of RNA, such as mRNA, suitable for administration to a mammal, such as a human. In various examples, the isolated nucleic acid is or comprises an mRNA having an open reading frame encoding a single domain antibody or an antigen binding molecule provided herein. mRNA vaccines are described, for example, in International Patent Application Nos. PCT/US2015/027400 and PCT/US2016/044918, herein incorporated by reference in their entirety. The mRNA delivery of antibodies, such as single domain antibodies, is further described in PCT/US2018/037918, which is also incorporated by reference herein.

It will be appreciated that nucleic acid, and more particularly mRNA, delivery of single domain antibodies (and antigen binding molecules comprising such single domain antibodies) provides a unique therapeutic alternative to peptide-based or DNA-based methods of administering such agents. When the mRNA is delivered to a cell, the mRNA will be processed into a polypeptide or peptide by the intracellular machinery which can then process the polypeptide or peptide into the single domain antibodies or antigen binding molecules capable of binding a viral protein or antigen of a norovirus on or in a virally-infected cell or biological fluid of the subject.

Compositions comprising isolated nucleic acids or polynucleotides that encode the single domain antibodies or antigen binding molecules described herein, or fragments, variants or derivatives thereof that may be used for such methods, are also contemplated by the present disclosure. For such examples, the composition suitably comprises a delivery agent, such as a nanoparticle, as are known in the art. In various examples, the nanoparticle has a mean diameter of 50-200 nm. In some examples, the composition comprising the mRNA polynucleotide (e.g., an mRNA polynucleotide having an open reading frame that encodes a single domain antibody or an antigen binding molecule described herein) is formulated in a lipid nanoparticle.

### Methods of Inactivating Norovirus In Vitro

The present disclosure further envisages use of the single domain antibodies described herein for decontaminating or disinfecting, for example, surfaces or substrates that may be contaminated with a norovirus. To this end, it is well known that norovirus can be spread indirectly by way of human contact with such contaminated substrates and surfaces, such as contaminated body surfaces, environmental surfaces, food and water. In this regard, the single domain antibodies described herein may be essentially functional as a disinfectant agent or an antiviral agent.

Accordingly, in one form, the present disclosure provides a method for inactivating or neutralising a norovirus associated with a substrate or surface (e.g., in and/or on the substrate or surface), said method including the step of contacting the substrate with an effective amount of the single domain antibody, the antigen binding molecule or the composition provided herein to thereby inactivate or neutralise the norovirus associated with the substrate or surface.

In particular examples, the surface or substrate is a body surface, such as of a subject currently infected with a norovirus or a subject in contact with such an infected subject.

In other examples, the surface or substrate is an environmental surface or substrate.

In view of the foregoing, the composition provided herein may be considered a disinfectant composition for inactivating a norovirus associated with a substrate or surface. Such disinfectant compositions may comprise one or more additional components with disinfectant properties in relation to viruses, such as an alcohol, a protease, an RNase, a detergent and a disinfectant (e.g., sodium hypochlorite).

So that preferred embodiments of the present disclosure may be fully understood and put into practical effect, reference is made to the following non-limiting examples.

### EXAMPLE

The current Example sought to identify nanobodies that efficiently prevent GII.4 and GII. 17 virus-like particles (VLPs) from binding to HBGAs and determine the nanobody binding site using X-ray crystallography. A number of nanobodies that have HBGA blocking capacities were identified and four nanobodies completely blocked the GII.4 and GII.17 HBGA binding sites.

### Materials and Methods

### Norovirus P Domain Production

Norovirus GII.4 Sydney-2012 (JX459908), GII.10 Vietnam 026 (AF504671), and GII.17 Kawasaki308 (LC037415) P domain were produced as previously described (9). In brief, the P domain gene was cloned into the pMal-c2X vector, followed by transformation into E. coli BL21 (DE) cells. Cells were grown in LB medium at 37° C. until cells reached an optical density (OD₆₀₀) of 0.6 and then induced with 0.7 mM IPTG (isopropyl thio-β-D-galactopyranoside) for 18 h at 22° C. Cells were harvested by centrifugation and lysed by sonication. The fusion P domain-MBP protein was purified using a Ni-NTA column and the MBP tag was cleaved from the P domain using HRV3C protease at 4° C. After another round of Ni-NTA chromatography, the cleaved P domain was dialyzed in gel filtration buffer (GFB: 25 mM Tris-HCl [pH 7.6] and 300 mM NaCl), purified by size exclusion chromatography, concentrated to ~4 mg/mL, and stored at 4° C.

### Norovirus VLP Production

The capsid gene of norovirus GI.1 (AY502016.1), GI.11 (AB058547), GII.1 (U07611), GII.4c (36), GII.4 CHDC-1974 (ACT76142), GII.4 Saga-2006 (AB447457), GII.4 Yerseke-2006a (EU876887), GII.4 Sydney-2012, GII.10 Vietnam 026, and GII.17 Kawasaki308 were cloned into a baculovirus expression system as previously described (37). Briefly, the VLPs secreted into the cell medium were separated from Hi5 insect cells by low-speed centrifugation, concentrated by ultracentrifugation at 30,000 rpm at 4° C. for 2 h (Beckman Ti45) and resuspended in PBS. The VLPs were purified by CsCl equilibrium gradient ultracentrifugation at 45,000 rpm at 15° C. for 18 h (Beckman SW-55) and fractions containing VLPs were pelleted by ultracentrifugation and resuspended in PBS to remove residual CsCl. Fractions were confirmed using EM and homogenous particles were pooled and concentrated to 2-10 mg/ml in PBS (pH 7.4). The VLP samples were applied to EM grids, washed once in distilled water, stained with 0.75% uranyl acetate, and examined using EM (Zeiss EM 910).

### Nanobody Production

The nanobody libraries were generated at the VIB Nanobody Service Facility with the approval of the ethics commission of Vrije Universiteit, Brussels, Belgium. Briefly, alpacas were injected subcutaneously with GII.4 Sydney-2012 or GII.17 Kawasaki308 VLPs. A VHH library was constructed and screened for the presence of antigen specific nanobodies. Nine nanobodies were selected based on sequence variation in the complementarity-determining regions (CDRs). These nine nanobodies (GII.4: NB-30, NB-53, NB-56, NB-76, and NB-82; and GII.17: NB-2, NB-7, NB-34, and NB-45) were subcloned into a pHEN6C expression vector and expressed in E. coli WK6 cells overnight at 28° C. Expression was induced with 0.7 mM IPTG at an OD₆₀₀ of 0.9. The cells were harvested by centrifugation and the nanobodies were extracted from the periplasm. After removing cell debris by centrifugation, the supernatant containing the nanobodies was collected. The His-tagged nanobodies were first purified using Ni-NTA chromatography and then, after dialysis into GFB, size-exclusion chromatography was performed. Nanobodies were concentrated to 2-3 mg/mL and stored in GFB at 4° C.

### Nanobody Reactivities Using ELISA

The nanobody reactivities against norovirus VLPs were determined using a direct ELISA as previously described (34). Microtiter plates (Maxisorp, Denmark) were coated with 100 μL (2 μg/mL) of GII.4 or GII.17 VLPs in PBS (pH 7.4). Wells were washed three times with PBS containing 0.1% Tween 20 (PBS-T) and then blocked with 300 μL of PBS containing 5% skim milk (PBS-SM) for 1 h at room temperature (RT). After washing, 100 μL of serially diluted nanobodies in PBS (from ~10 μM) were added to each well. The wells were washed and then 100 μL of a 1:3,000 dilution of secondary HRP-conjugated anti-His IgG (Sigma) was added to wells for 1 h at 37° C. After washing, 100 μL of substrate o-phenylenediamine and H₂O₂ was added to wells and left in the dark for 30 min at RT. The reaction was stopped with the addition of 50 μL of 3 N HCl and the absorbance was measured at 490 nm (OD₄₉₀). The final OD₄₉₀=sampleₘₑₐₙ minus PBSₘₑₐₙ (~ 0.05). A cutoff limit was set at OD₄₉₀>0.15, which was ~3 times the value of the negative control (PBS). All experiments were performed in triplicate.

### Nanobody HBGA Inhibition

A surrogate HBGA neutralization assay using porcine gastric mucin (PGM) was performed as described previously (19, 26, 38). Briefly, ELISA plates were coated with 10 μg/mL of PGM (Sigma Aldrich) for 1 h at 37° C. Coated plates were washed and blocked with 5% skim milk in PBS for 1 h at RT. The nine nanobodies were serially diluted from a starting concentration of 100 μg/mL and added to GII.4 and GII. 17 VLPs with a final concentration of 1 μg/mL and 2 μg/mL, respectively. After 30 min incubation at RT, the VLP-NB mixture was added to the wells and incubated for 1 h at 37° C. The GII.4 VLPs were detected with a polyclonal rabbit anti-GII.4 antibody (34) and followed by HRP-conjugated anti-rabbit antibody, while GII.17 VLPs were detected using biotinylated nanobody-28 (NB-28) and HRP-conjugated streptavidin. Plates were then developed with o-phenylenediamine and H₂O₂ in the dark at RT. After 30 min, the reaction was stopped with 6% (v/v) HCl, and absorption measured at OD₄₉₀. The binding of untreated VLPs was set as a reference value corresponding to 100% binding. The percentage of inhibition was calculated as [1-(treated VLP mean OD₄₉₀/mean reference OD₄₉₀)]×100. The half maximal inhibitory concentration (IC₅₀) value for inhibition was determined using GraphPad Prism (version 8.0). All experiments were performed in triplicates and the mean and standard deviation calculated.

### Nanobody Isothermal Calorimetry Measurements

Isothermal calorimetry (ITC) experiments were done using an ITC-200 (Malvern). Samples were prepared in PBS and filtered prior to the experiments. Titrations were performed at 25° C. by injecting consecutive (2-3 μL) aliquots of nanobodies (100 μM) into GII.4 Sydney-2012 or GII.17 Kawasaki308 P domains (10-20 μM) in 120 s intervals. Injections were performed until saturation was achieved. To correct for heats of dilution from titrants control experiments were performed by titrating nanobodies into PBS. The heat associated with the control titration was subtracted from raw binding data prior to fitting. The data was fitted using a single set-binding model (Origin 7.0 software). Nanobody binding sites on the P domains were assumed to be identical. All ITC experiments were performed in triplicate with the average and standard deviation calculated.

### Purification and Crystallization of Norovirus P Domain and Nanobody Complexes

The P domain (GII.4 Sydney-2012, GII.10, or GII.17 Kawasaki308) and nanobody were mixed in a 1:1.4 molar ratio and incubated at 25° C. for ~90 min. The complex was purified by size exclusion chromatography using a Superdex-200 column and concentrated to 2.8-10 mg/mL. Complex crystals were grown using hanging-drop vapor diffusion method at 18° C. for ~6-10 days. Crystallization of the complexes was achieved using the following conditions: GII.4-NB-30 (0.2 M calcium chloride and 20% [w/v] PEG3350); GII.4-NB-53 (12% [w/v] PEG20000 and 0.1 M MES [pH 6.5]); GII.4-NB-56 (10% [w/v] PEG8000 and 0.1 M MES [pH 6.0]); GII.4-NB-76 (17% [w/v] PEG4000, 0.0095 M HEPES [pH 7.5], 8.5% [v/v] isopropanol, and 15% glycerol); GII.4-NB-82 (0.8 M ammonium sulfate, 0.1 M bicine [pH 9]); GII.10-NB-34 (1 M lithium chloride, 10% [w/v] PEG6000, and 0.1 M citric acid [pH 5]); GII. 17-NB-2 (0.1 M bicine [pH 9] and 30% [w/v] PEG6000); GII.17-NB-7 (0.8 M ammonium sulfate and 0.1 M citric acid [pH 4]); and GII.17-NB-45 (1.6 M ammonium sulfate, 0.08 M sodium acetate, and 20% [v/v] glycerol). Prior to flash-freezing in liquid nitrogen, crystals were transferred to a cryoprotectant containing the mother liquor in 30% ethylene glycol.

### X-Ray Data Collection, Structure Solution, and Refinement

X-ray diffraction data were collected at the Deutsches Elektronen-Synchrotron (DESY: PETRA III beamlines: P13 and P14), Germany and processed with XDS (39) and MOSFLM. Structures were solved by molecular replacement in PHASER (40). Structures were refined in multiple rounds of manual model building in COOT (41) and refined with PHENIX (42). Structures were validated with Procheck (43) and Molprobity (44). PISA software was used to determine binding interfaces and calculate surface area (45). Binding interfaces and interactions were analyzed using PDBePISA online server (https://www.ebi.ac.uk/pdbe/pisa/) (45) and PyMOL (version 1.2), with hydrogen bond distances ~2.36-3.88 Å and electrostatic distances ~2.56-3.89 Å. Water-mediated interactions were excluded from the analysis. Figures and protein contact potentials were generated using PyMOL. Atomic coordinate and structure factors of the X-ray crystal structures were deposited in the Protein Data Bank (8EMY, GII.4-NB-82; 8EMZ, GII.17-NB-2; 8EN0, GII.17-NB-7, 8EN1, GII.4-NB-30; 8EN2, GII.10-NB-34; 8EN3, GII.17-NB-45; 8EN4, GII.4-NB-53; 8EN5, GII.4-NB-56; and 8EN6, GII.4-NB-76).

### Results

In this study, nine norovirus-specific nanobodies were produced in alpaca against norovirus VLPs representing two clinically important GII genotypes, GII.4 Sydney-2012 (NB-30, NB-53, NB-56, NB-76, and NB-82) and GII.17 Kawasaki308 (NB-2, NB-7, NB-34, and NB-45). These nine nanobodies were chosen based on the amino acid variation in the CDRs with an overall sequence identity ranging between 62-82%. In order to compare these nine nanobodies with previously reported norovirus nanobodies and monoclonal antibodies, the binding interactions, HBGA blocking capacities, and X-ray crystal structures were analyzed using established methods (34, 35, 46, 47).

### Nanobody Binding Specificities

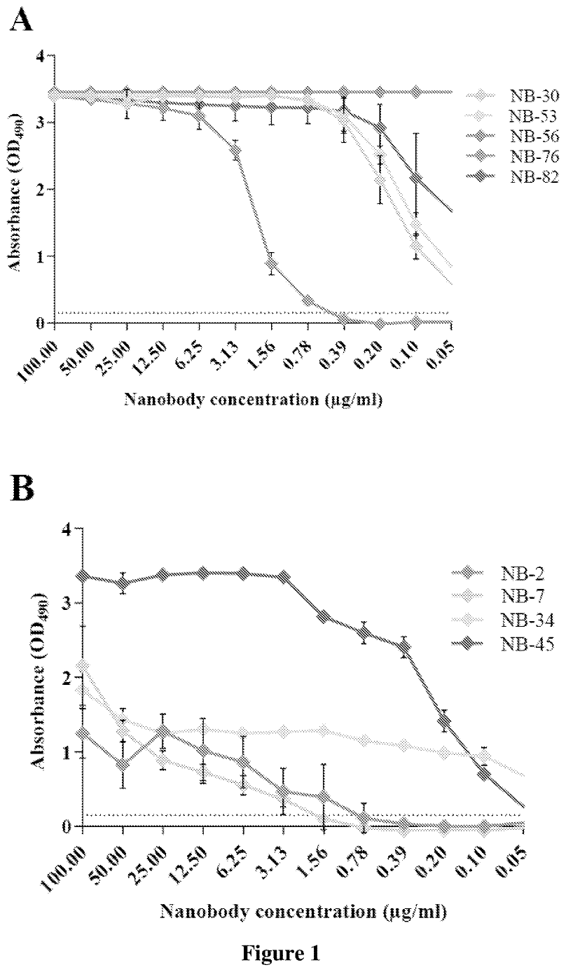

The GII.4 and GII.17 nanobody binding specificities were initially analyzed using a direct ELISA with the corresponding immunization VLP antigens as previously described for GI.1 and GII.10 nanobodies (34, 47). All five GII.4 nanobodies bound to the GII.4 Sydney-2012 VLPs (FIG. 1A). NB-56 showed the strongest binding and at the lowest concentration (0.05 μg/mL) with a maximum signal (OD₄₉₀=3.5). NB-82 also exhibited strong binding over the dilution range with an OD₄₉₀=2.0 at the lowest nanobody concentration. Likewise, NB-30 and NB-53 showed strong binding with an OD₄₉₀=0.8 and 1.0 at the lowest nanobody concentration, respectively. NB-76 reached the cut-off at a concentration of 0.38 μg/mL. All four GII. 17 nanobodies bound to the GII.17 Kawasaki308 VLPs (FIG. 1B). The strongest binders were NB-34 and NB-45, with an OD₄₉₀=0.8 and 0.6 at the lowest nanobody concentration, respectively. NB-7 and NB-2 reached the cut-off at a concentration of 1.56 and 0.78 μg/mL, respectively.

### Nanobody Cross-Reactivity

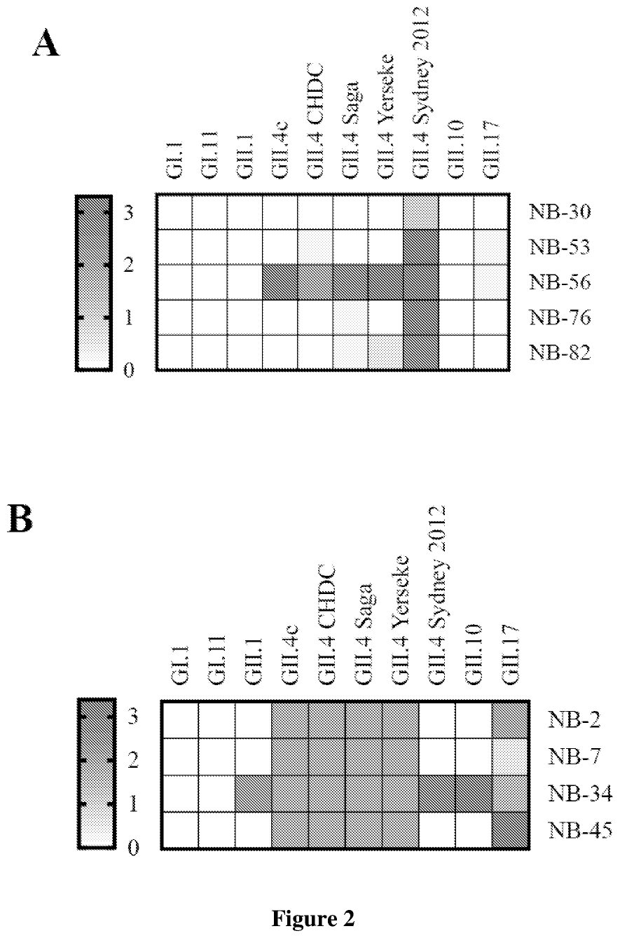

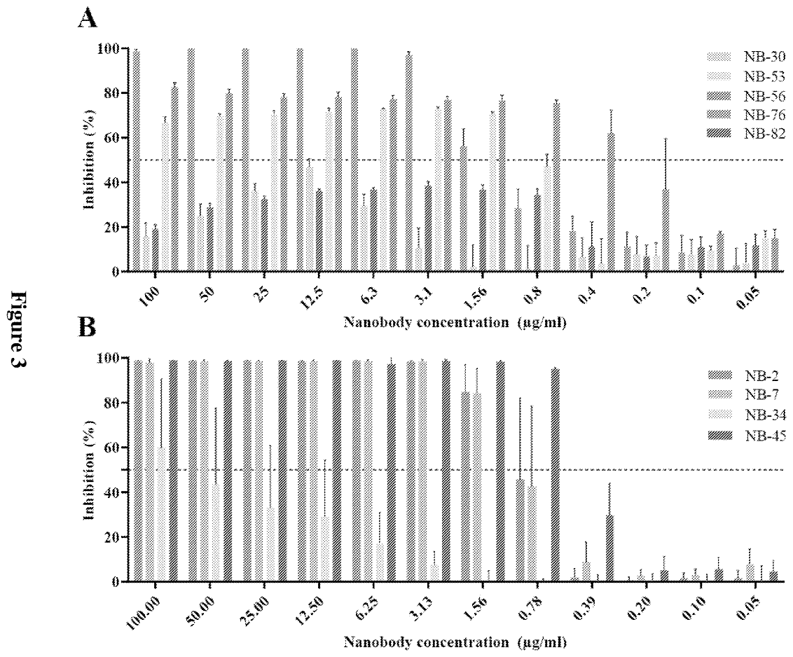

Nanobody cross-reactivities were analyzed with a panel of VLPs from GI and GII (i.e., GI.1, GI.11, GII.1, GII.4, GII.10, and GII.17) and five different GII.4 variants (89.61-98.7% amino acid identity) as described previously (48). NB-30, NB-76, and NB-82 were mainly GII.4 Sydney-2012 specific, whereas NB-56 was able to bind strongly to all GII.4 variants (FIG. 2A). NB-53 and NB-56 showed weak cross-reactivity with GII.17 VLPs. Three GII.17 nanobodies (NB-2, NB-7, and NB-45) were genotype specific, whereas NB-34 cross-reacted strongly with GII.1, GII.4, GII.10, and GII.17 VLPs (FIG. 2B).

### HBGA Blocking Properties of Nanobodies

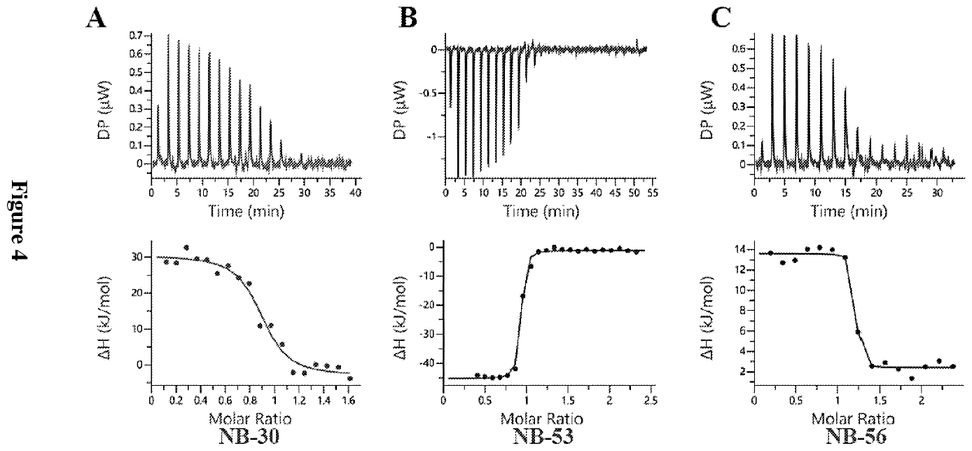

In order to determine the HBGA blocking potential of the nanobodies, a well-established surrogate HBGA neutralization assay was performed using GII.4 and GII.17 VLPs (19, 34, 47, 49, 50). For GII.4 nanobodies, it was found that NB-56 had the strongest HBGA blocking capacity with a half-maximal inhibitory concentration (IC₅₀)=0.26 μg/mL, followed by NB-53 (IC₅₀=0.76 μg/mL) and NB-76 (IC₅₀=1.42 μg/mL) (FIG. 3A). The IC₅₀ for NB-30 and NB-82 could not be calculated since the HBGA inhibition was below 50% for all dilutions. For GII.17 nanobodies, NB-45 showed the strongest blocking capacity with an IC₅₀=0.46 μg/mL, followed by NB-2 (IC₅₀=0.85 μg/mL), NB-7 (IC₅₀=0.88 μg/mL), and NB-34 (IC₅₀=15.33 μg/mL) (FIG. 3B).

### Thermodynamic Properties

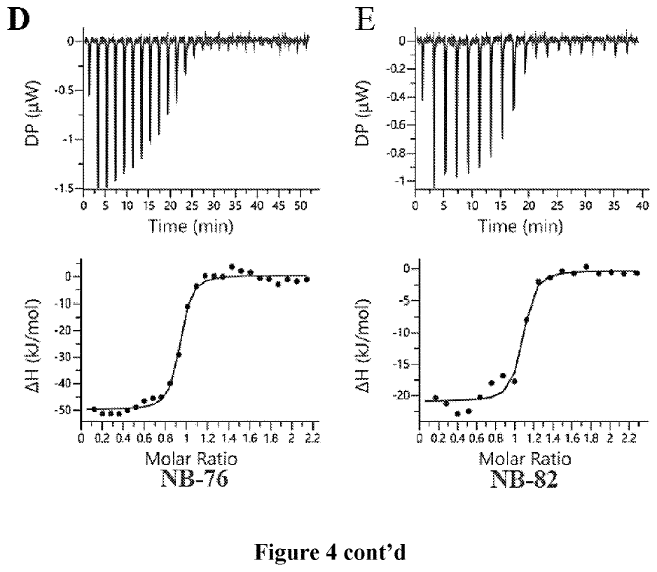

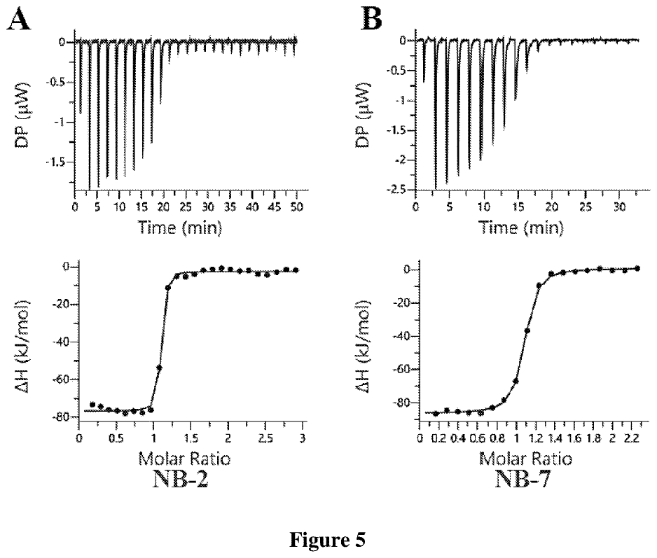

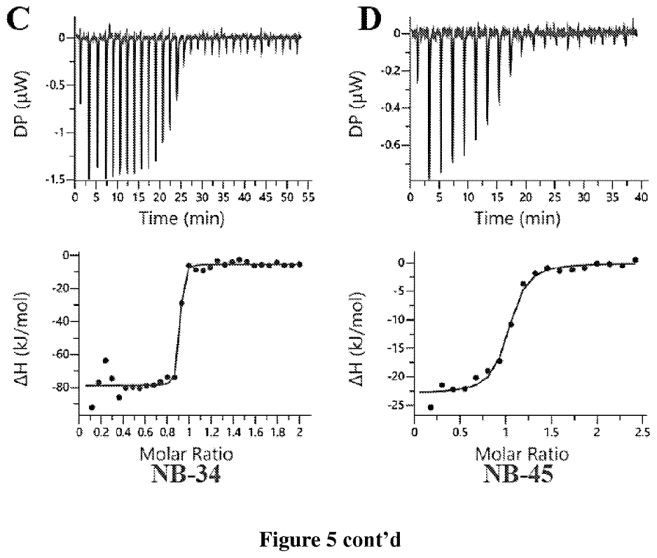

The thermodynamic properties of nanobodies binding to GII.4 Sydney-2012 and GII.17 Kawasaki308 P domains were analyzed using ITC. Titrations were performed at 25° C. by injecting consecutive aliquots of 100 μM of nanobodies into 15 μM of P domain (FIGS. 4 and 5). The stoichiometry indicated the binding of one nanobody molecule per P domain monomer. Most of the nanobodies (NB-53, NB-76, NB-82, NB-2, NB-7, NB-34, and NB-45) exhibited exothermic binding, while NB-30 and NB-56 were characterized by a positive enthalpy change associated with endothermic type of reaction. The binding constants, K_{d} (dissociation constant), ΔH (heat change), ΔS (entropy change), −TΔG (change in free energy) are summarized in Table 1. All the nanobodies bound tightly to the P domain, with the K_{d} ranging between 3.8 nM to 180 nM.

### X-Ray Crystal Structures of Norovirus P Domain and Nanobody Complexes

The structures of the GII.4 Sydney-2012 P domain in complex with NB-30, NB-53, NB-56, NB-76, and NB-82; and GII.17 Kawasaki308 P domain in complex with NB-2, NB-7, and NB-45 were solved using X-ray crystallography (Tables 2 and 3). In addition, the GII.10 P domain in complex with NB-34 was solved using X-ray crystallography to investigate NB-34 cross-reactivity at the atomic level (Table 3). The overall structure of the P domains in the complexes was comparable to the unbound P domains with minimal loop movements upon nanobody engagement. All nanobodies had typical immunoglobulin folds and the nanobody CDRs primarily interacted with the P domains. The nine nanobodies bound to the P domains at three distinct regions, termed side (NB-30, NB-53, NB-56, and NB-82), bottom (NB-34), and top of P domain (NB-2, NB-7, NB-45, and NB-76).

### Nanobodies Binding to the Side of the P Domain

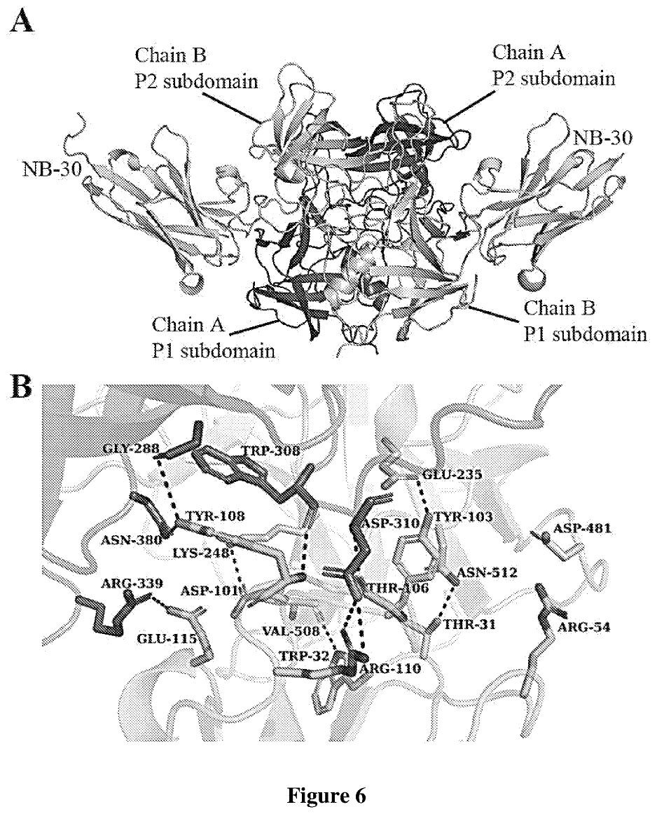

The GII.4 P1 subdomain comprises of residues 224-274 and 418-530, whereas the P2 subdomain is between residues 275-417 (8). It was found that NB-30 bound to the side of the GII.4 P domain dimer (FIG. 6A). A network of direct hydrogen bonds was formed between NB-30 and both P domain monomers (FIG. 6B). Nine GII.4 P domain residues (chain A: GLY-288, TRP-308, ASP-310, ARG-339, and ASN-380; chain B: GLU-235, LYS-248, VAL-508, and ASN-512) formed eleven hydrogen bonds with NB-30. In this location, NB-30 interacted with both GII.4 P1 and P2 subdomain residues.

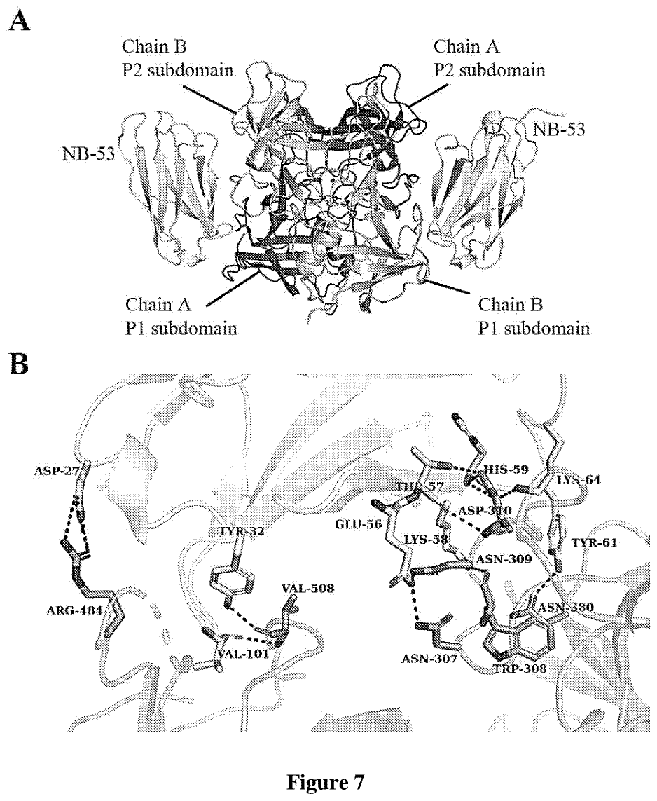

It was also discovered that NB-53 bound to the side of the GII.4 P domain (FIG. 7A). Seven GII.4 P domain residues located on both P domain monomers formed 15 direct hydrogen bonds with NB-53 (chain A: ARG-484 and VAL-508; chain B: ASN-307, TRP-308, ASN-309, ASP-310, and ASN-380) (FIG. 7B). Interestingly, NB-53 and NB-30 binding sites were at a similar location on the side of the P domain. Moreover, several P domain residues interacting with NB-30 and NB-53 were shared (i.e., TRP-308, ASP-310, ASN-380, and VAL-508).

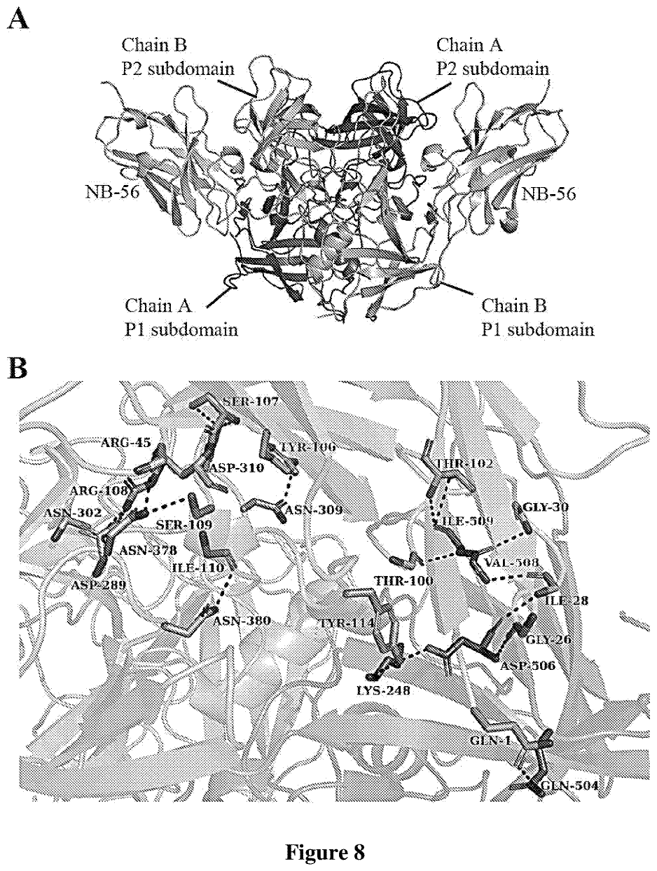

Comparable to NB-30 and NB-53 binding footprint, it was found that NB-56 bound on the side of the GII.4 P domain and interacted with both P domain monomers (FIG. 8A). Eleven P domain residues on P1 and P2 subdomains (chain A: LYS-248, GLN-504, ASP-506, VAL-508, and ILE-509; chain B: ASP-289, ASN-302, ASN-309, ASP-310, ASN-378, and ASN-380) formed 20 direct hydrogen bonds with NB-56 (FIG. 8B). Several GII.4 P domain residues interacting with NB-56 and NB-53 (ASN-380 and VAL-508) and NB-30 (LYS-248, ASP-310, ASN-380, and VAL-508) were shared.

Interestingly, NB-30, NB-53, and NB-56 and previously determined Nano-32 bound to the side of the P domain at a similar location (FIG. 9) and three nanobodies (NB-53, NB-56, and Nano-32) showed HBGA blocking potential (FIG. 3) (34). Moreover, a neutralizing monoclonal antibody (termed A1431) isolated from a patient immunized with the GII.4c VLP vaccine also bound to the side of the P domain at a region nearby this common nanobody binding site (FIG. 9) (19).

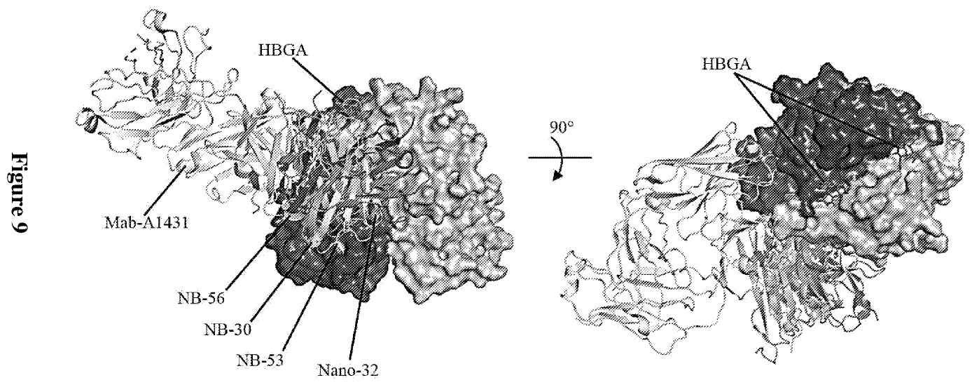

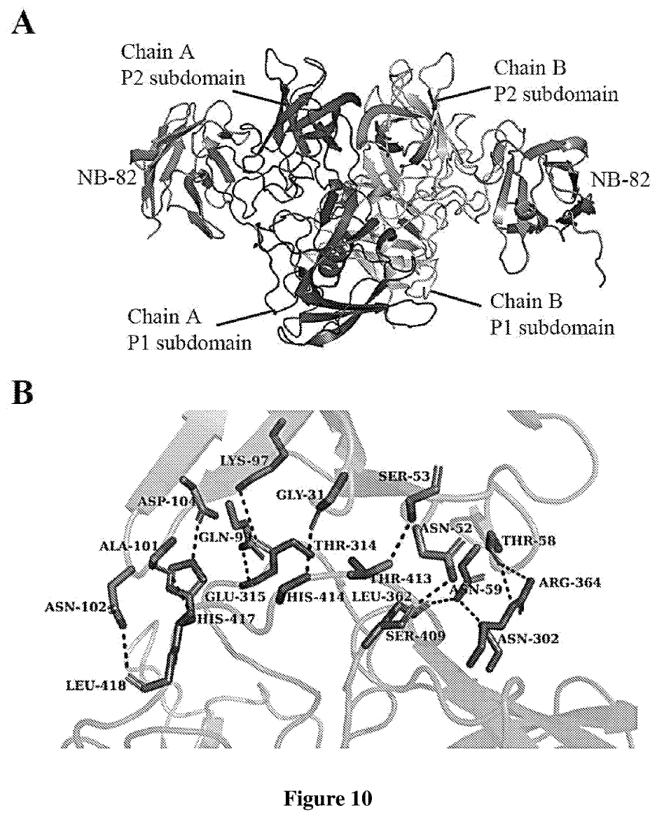

It was demonstrated that NB-82 bound on the side of the GII.4 P domain and interacted with only one P domain monomer (FIG. 10A). Unlike NB-30, NB-53, and NB-56, which were positioned down-towards the P domain, NB-82 was positioned across the P domain. Moreover, the NB-82 binding footprint was distinct from other GI.1 and GII.10 nanobodies (34, 35, 47). Ten P domain residues mainly located on the P2 subdomain formed 13 direct hydrogen bonds with NB-82 (chain A: ASN-302, THR-314, GLU-315, LEU-362, ARG-364, SER-409, THR-413, HIS-414, HIS-417, and LEU-418) (FIG. 10B).

### Nanobody Binding to the Bottom of the P Domain

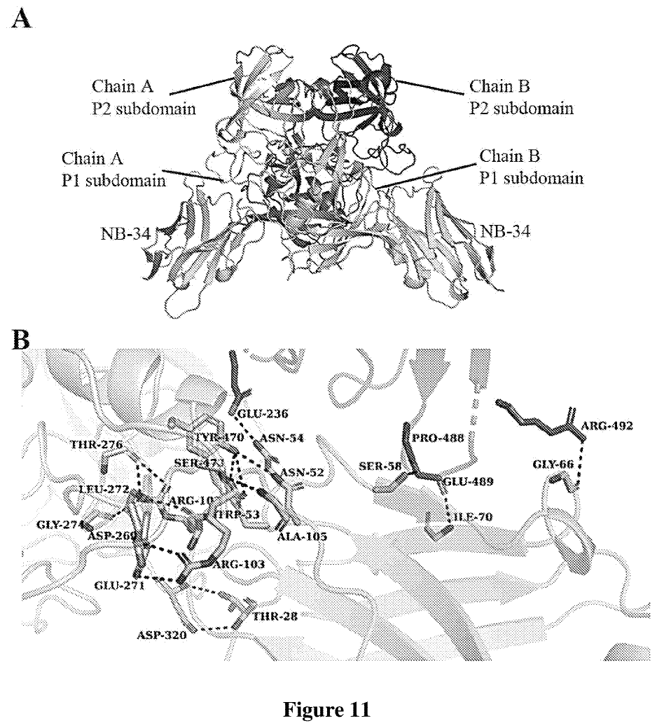

The X-ray crystal structure of GII.10 P domain with NB-34 was determined to explain nanobody cross-reactivity binding interactions at the atomic level. The GII.10 P1 subdomain is located between residues 222-277 and 427-549, while the P2 subdomain is between residues 278 and 426 (9). NB-34 bound to the bottom of the GII.10 P domain (FIG. 11A). Twelve P domain residues mostly located in the P1 subdomain formed 16 direct hydrogen bonds with NB-34 (chain A: ASP-269, GLU-271, LEU-272, GLY-274, THR-276, ASP-320, TYR-470, and SER-473; chain B: GLU-236, PRO-488, GLU-489, and ARG-492) (FIG. 11B).

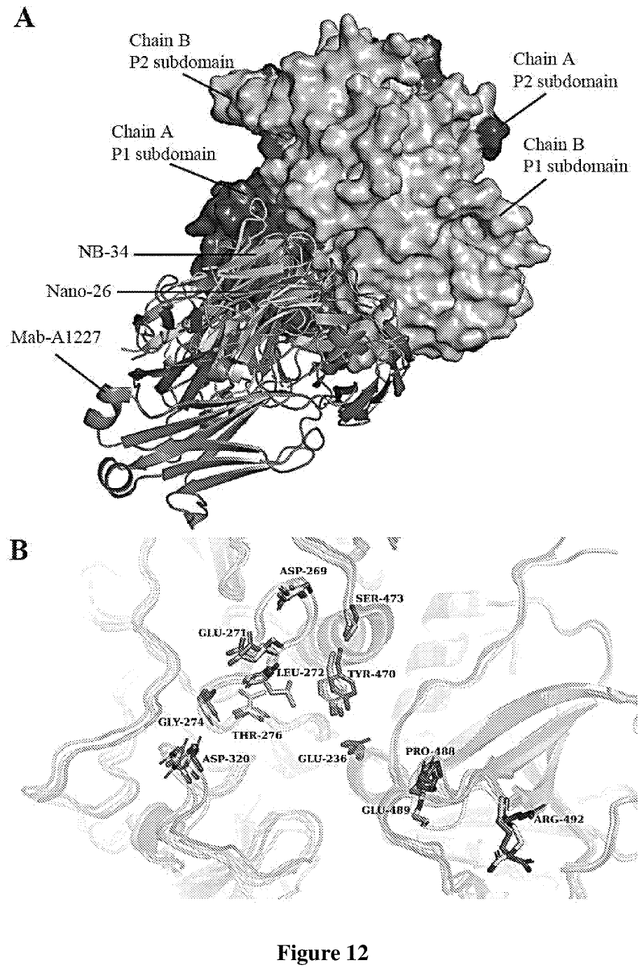

The ELISA showed that NB-34 cross-reacted against GII.1, GII.4, GII.10, and GII.17 VLPs (FIG. 2). NB-34 binding site was almost identical to a broadly-reactive diagnostic nanobody, termed Nano-26 and nearby a binding site of broadly-reactive monoclonal antibody (A1227) which was isolated from a patient immunized with the GII.4c VLP vaccine (FIG. 12A) (19, 34, 51). Superposition of GII.1, GII.4, and GII.17 apo X-ray crystal structures onto the GII.10 Vietnam026 P domain NB-34 complex revealed that the residues that formed direct hydrogen bonds with NB-34 were comparatively conserved among these four genotypes despite numerous amino acid insertions and deletions among these genotypes (FIG. 12B). Indeed, seven P domain residues that interacted with Nano-26 also formed direct hydrogen bonds with NB-34 (i.e., ASP-269, GLU-271, LEU-272, GLY-274, THR-276, TYR-470, and PRO-488).

### Nanobodies Binding to the Top of the P Domain

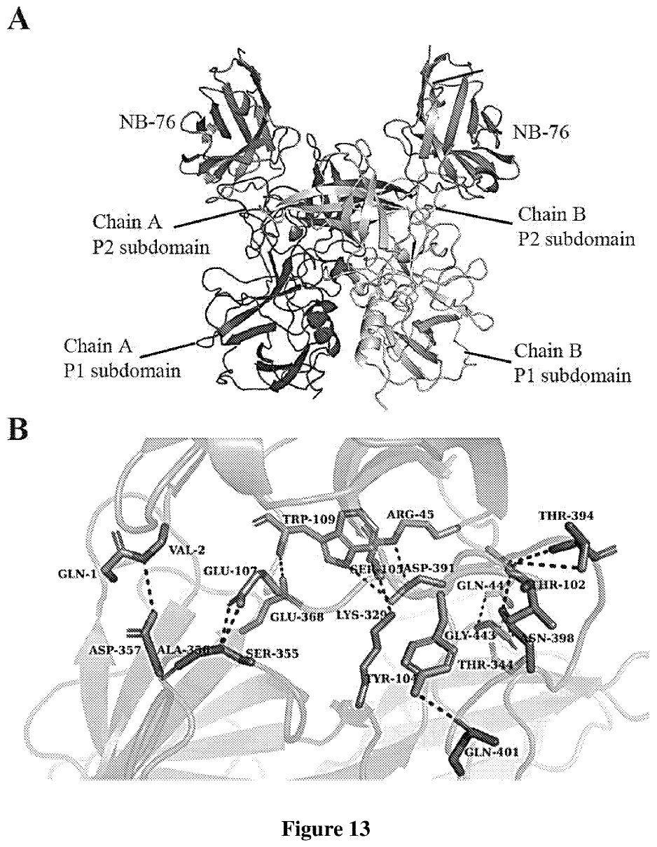

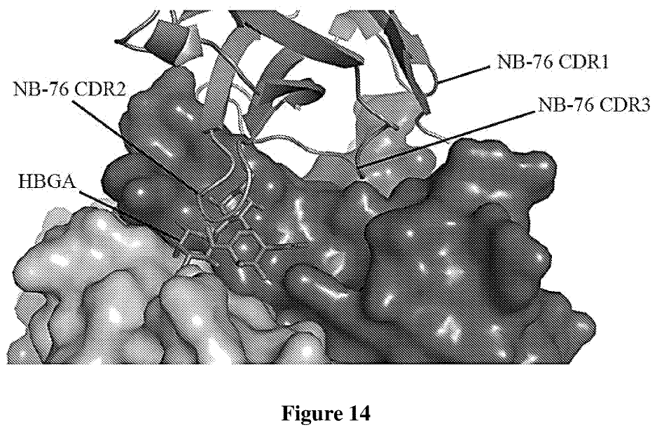

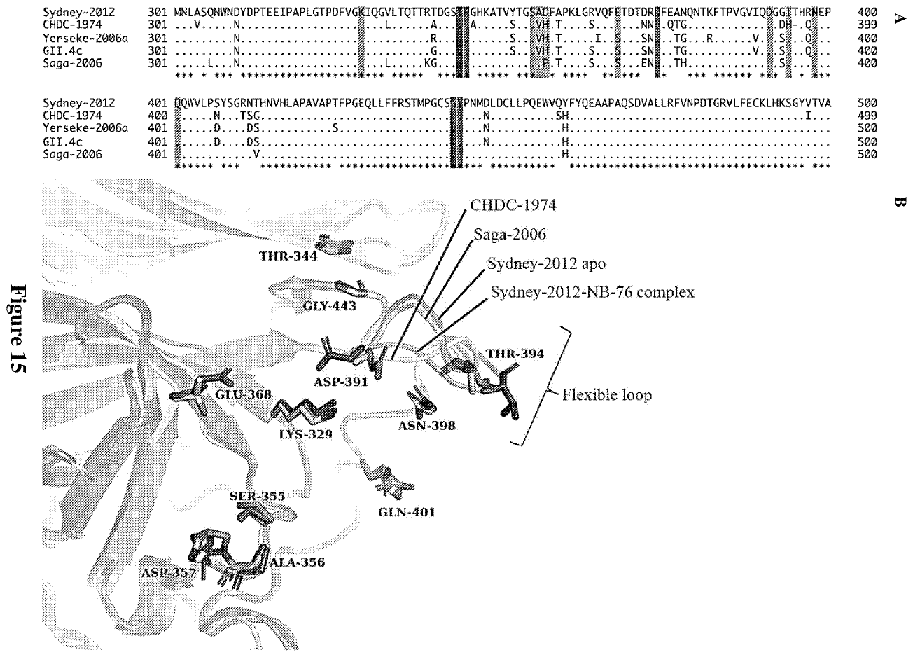

It was found that NB-76 bound to the top of the GII.4 P2 subdomain (FIG. 13A). Eleven P domain residues from both P domain monomers formed 14 direct hydrogen bonds with NB-76 (chain A: LYS-329, SER-355, ALA-356, ASP-357, GLU-368, ASP-391, THR-394, ASN-398, GLN-401, and GLY-443; chain B: THR-344) (FIG. 13B). Superposition of the previously determined GII.4 Sydney-2012 P domain A-trisaccharide complex onto the GII.4 Sydney-2012 P domain NB-76 complex revealed that part of NB-76 CDR2 (LYS-43 and GLN-44) covered the fucose moiety from the A-trisaccharide and was close to the second moiety of the HBGA molecule (FIG. 14). The five GII.4 P domain residues that commonly bind HBGAs include ASP-374, ARG-345, THR-344, GLY-443, and TYR-444. Two of these common HBGA binding residues also bound NB-76 (THR-344 and GLY-443) (FIG. 15A). Structural alignment of CHDC-1974 and Saga-2006 P domains onto Sydney-2012 P domain (apo and NB-76 complex) confirmed the amino acid substitutions on the P domains at the NB-76 binding residues (FIG. 15B). This structural analysis also revealed a slight loop movement (between residues 391-398) upon NB-76 engagement.

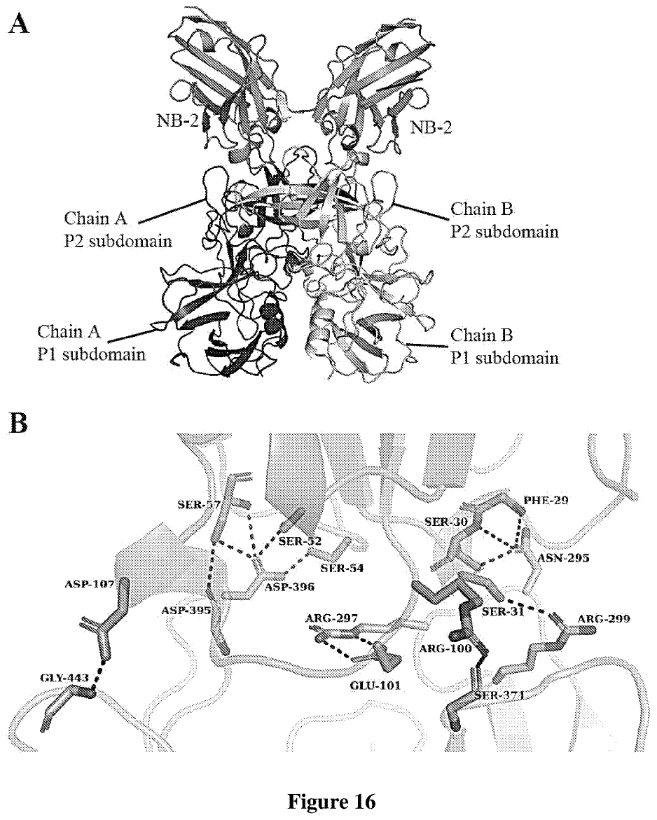

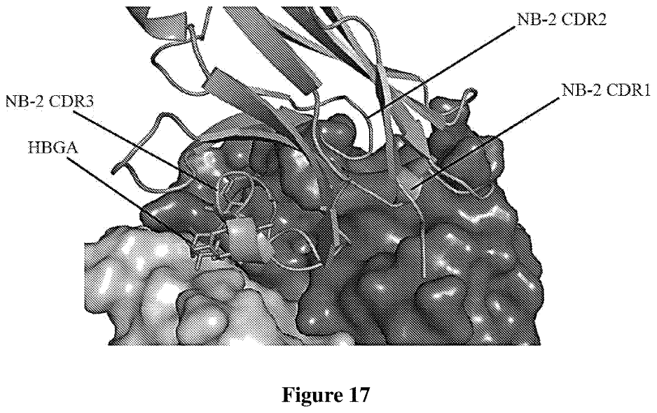

The GII.17 P1 subdomain contains residues 225-275 and 419-540, whereas the P2 subdomain is between residues 276-418 (52). It was found that NB-2 bound on the top of the GII.17 P2 subdomain and interacted with one of the P domain monomers (FIG. 16A). Seven P domain residues (chain A: ASN-295, ARG-297, ARG-299, SER-374, ASP-395, ASP-396, and GLY-443) formed 13 direct hydrogen bonds with NB-2 (FIG. 16B). Superposition of the GII.17 Kawasaki308 P domain A-trisaccharide complex onto the GII.17 Kawasaki308 P domain NB-2 complex showed that part of NB-2 CDR3 (PRO-106, ASP-107, and SER-108) covered the second moiety of the HBGA molecule (FIG. 17). In addition, GII.17 P domain interface loop residues (GLY-443) interacted with NB-2 and this GII.17 P domain residue (TYR-443) that commonly held HBGAs formed a direct hydrogen bond with NB-2 (ASP-107) (FIG. 17).

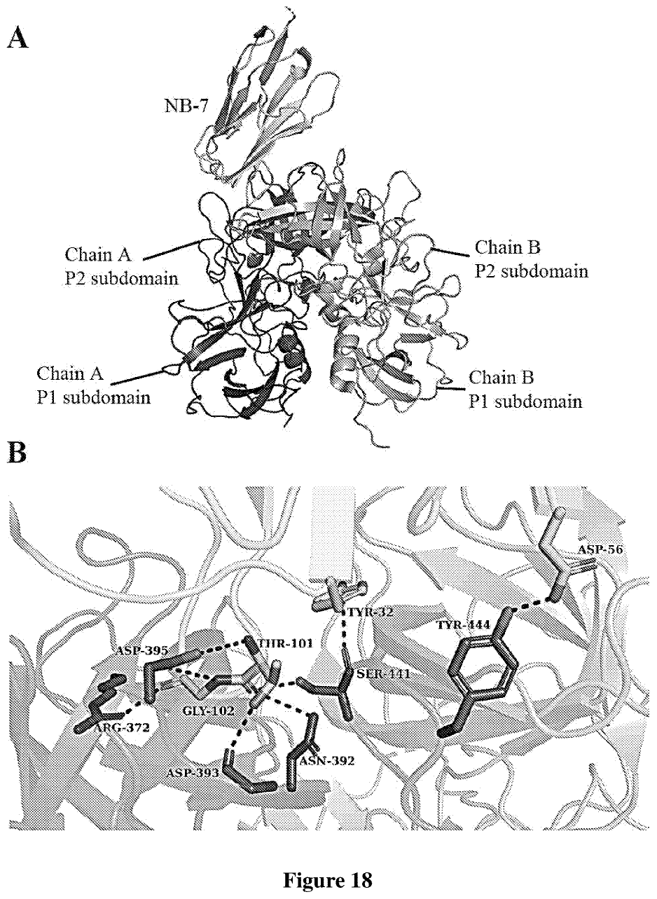

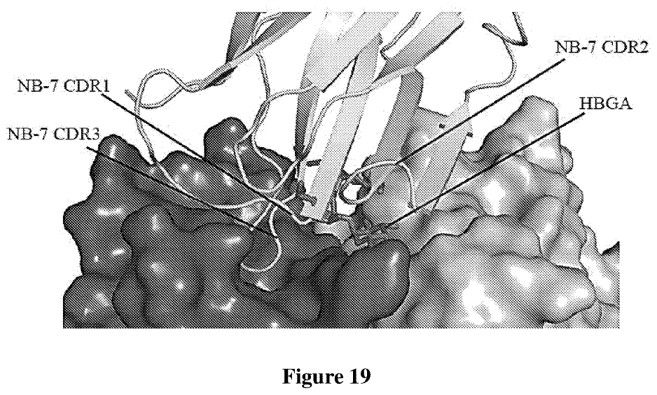

It was discovered that NB-7 also bound on the top of the GII. 17 P2 subdomain (FIG. 18A) and interacted with one P domain monomer. Six P domain residues formed eight direct hydrogen bonds with NB-7 (chain A: ARG-372, ASN-392, ASP-393, ASP-395, SER-441, and TYR-444) (FIG. 18B). It was found that several NB-7 residues (TYR-32 and ARG-33 on CDR2) covered the second moiety of the HBGA molecule, while TYR-99 side chain (CDR3) overlapped the third moiety of the HBGA molecule (FIG. 19). In addition, the GII. 17 P domain interface loop residues (SER-441 and TYR-444) interacted with NB-7 and one GII.17 P domain residue (TYR-444) that commonly held HBGAs formed a direct hydrogen bond with NB-7 (residue GLU-46).

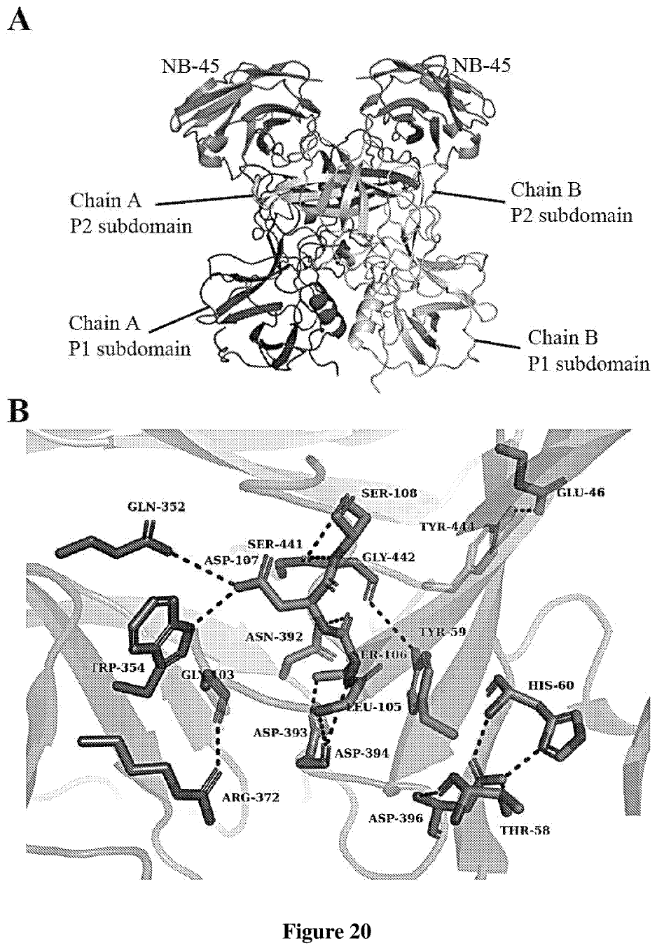

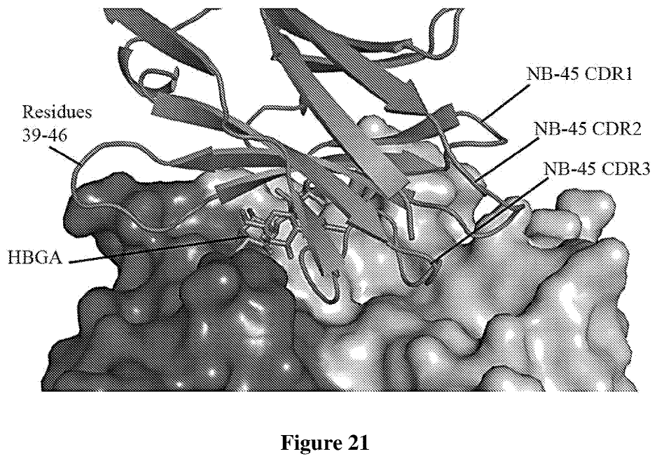

The present data also demonstrate that NB-45 bound on the top of the GII. 17 P2 subdomain and interacted with both P domain monomers (FIG. 20A). Thirteen GII.17 P domain residues formed 20 direct hydrogen bonds with NB-45 (chain A: GLN-352, TRP-354, ARG-372, ASN-392, ASP-393, ASP-394, ASP-396, SER-441, GLY-442, and TYR-444; chain B: ASN-295, GLN-296, and GLN-361) (FIG. 20B). Superposition of the GII.17 P domain A-trisaccharide complex onto the GII.17 Kawasaki308 P domain NB-45 complex revealed that NB-45 CDR2 and CDR3 surrounded the HBGA molecule and likely blocked access to the HBGA site on the P domain (FIG. 21). In addition, GII.17 P domain interface loop residues (SER-441, GLY-442, and TYR-444) interacted with NB-45 and one GII. 17 P domain residue (TYR-444) that commonly held HBGAs formed a direct hydrogen bond with NB-45 (residue GLU-46). Furthermore, two NB-45 residues (VAL-48 and ALA-49) clashed with the second moiety of HBGA, and the side chain of one NB-45 residue (SER-108) was in proximity (~1.7 Å) to the fucose moiety of HBGA.

### Discussion

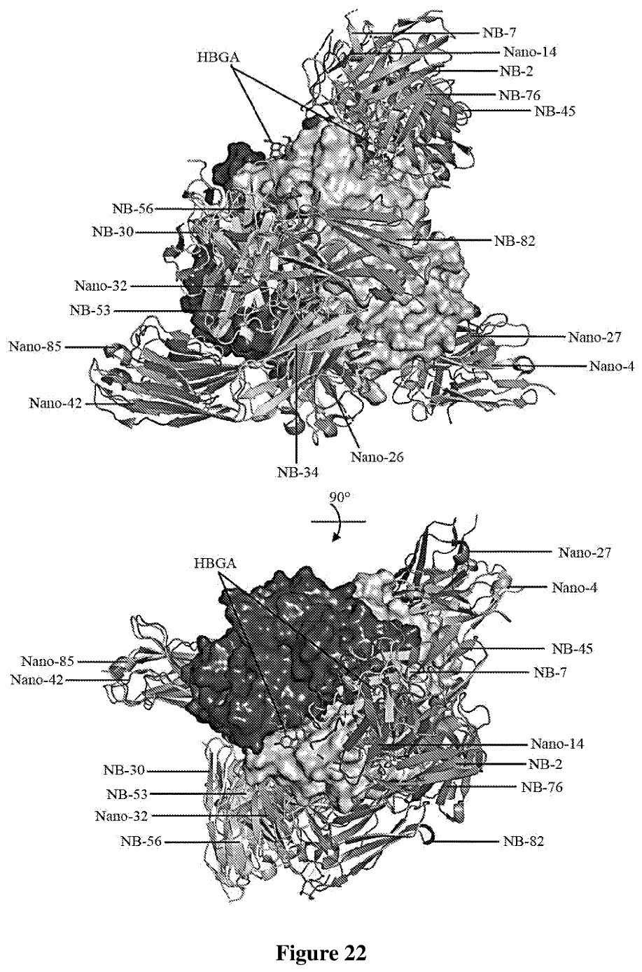

In this Example, a new panel of nanobodies against two major outbreak norovirus genotypes (i.e., GII.4 and GII.17) were developed with the intention of generating nanobodies that block access to the HBGA binding sites. Both genotypes have been extensively analyzed for HBGA binding interactions, inhibition studies, and capsid evolution (8, 13, 35, 53-68) as well clinical trials using GII.4 VLPs as candidate vaccines, reviewed in (15). The new nanobody structures have also been compared with previously generated nanobodies developed against the rarely detected GII.10 norovirus (34, 35), which has been extensively characterized in complex with HBGAs, HMOs, and citrate (9, 25-28). The present inventors have now characterized the X-ray crystallographic structures for 16 different GII P domain nanobody complexes (FIG. 22).

Five nanobodies (Nano-26, Nano-85, NB-34, NB-53, and NB-56) that bound to the bottom or side of the P domain indirectly prevented norovirus VLPs from binding to HBGAs (34). For Nano-85 and Nano-26 (developed against GII.10 VLPs), it was shown that the nanobody binding epitopes were positioned at a cryptic and vulnerable region located between the S and P domains (34, 35). It was found that Nano-85 and Nano-26 engagement disrupted VLP stability and resulted in VLP disassembly and aggregation as observed by electron microscopy (34, 35). Similarly, one recent study isolated a human norovirus IgA (antibody noro-320) from a norovirus-infected patient and showed that IgA engagement also led to virion aggregation observed by electron microscopy and neutralization in cell culture (69). Another monoclonal antibody (termed A1431) bound to the side of the P domain (FIG. 9) and was neutralizing in cell culture (19), presumably A1431 sterically blocked norovirus from attaching to large multivalent HBGAs found in PGM (19).

Interestingly, NB-34 closely mimicked Nano-26 binding site and both nanobodies were broadly reactive and interacted with mainly conserved P domain binding residues. However, the precise HBGA blocking mechanism of these nanobodies that bind to the side of the P domain (i.e., NB-34, NB-53, and NB-56) remains unclear, as VLP disassembly and aggregation after treatment with these nanobodies were not observed (data not shown). On the other hand, ubiquity of these side binding nanobodies and several monoclonal antibodies suggest that substantial flexibility in virus particles is functionally important for antibody- and nanobody-mediated recognition and/or neutralization (19, 34, 69). Indeed, the present inventors have previously identified a broadly reactive norovirus diagnostic IgG monoclonal antibody (termed 5B18) that bound at a highly conserved and occluded epitope at the bottom of the P domain (70). For these nanobodies and antibodies to interact with the virion, the P domains need to be rather flexible on the S domain. This suggests that for some antibody- or nanobody-recognition events the engagement might also result in virus neutralization by interfering with capsid stability that indirectly affects the HBGA binding site and/or sterically blocks HBGA engagement as was recently discovered with human norovirus nanobodies and monoclonal antibodies (19, 34, 35).

Four nanobodies (NB-76, NB-2, NB-7, and NB-45) that bound to the top of the P domain, completely obscured the GII.4 or GII.17 HBGA binding pockets, and blocked VLPs from binding to HBGAs. Compared to nanobodies and antibodies that bind to the side of the P domain and indirectly interfere with the HBGA pocket, these four nanobodies interacted with several P domain residues that commonly bind HBGAs and likely sterically obstructed HBGA engagement. Several other inhibitors that impeded the HBGA binding pocket and interacted with P domain residues that bound HBGAs include HMOs and citrate (25-28, 71). These natural compounds closely mimicked the HBGAs structures and might function as weak binding antivirals with millimolar affinities (25-28, 71). The present inventors have also previously identified a monoclonal antibody (termed 10E9) developed against GII.10 VLPs, that partially blocked the HBGA pocket and interacted with several HBGA binding residues (ARG-345 and TYR-444) (38). This monoclonal antibody also inhibited Sydney-2012 VLPs from binding to HBGAs in the surrogate HBGA neutralization assay and GII.4 norovirus replication in cell culture (38).

In summary, the present inventors have now identified four new nanobodies that directly impede the HBGA binding pockets for two major norovirus genotypes. The correlation between HBGA blocking potential using the surrogate HBGA neutralization assay and the norovirus cell culture has been supported in numerous studies, several of which included structural analysis of inhibitors (12, 19, 38, 69, 72).

TABLE 1

| P domain-Nanobody | K_{d} (nM) | ΔH (kJ/mol) | ΔG (kJ/mol) | +TΔS (kJ/mol) |
|---|---|---|---|---|
| GII.4-NB-30 | 180 ± 50 | 33.5 ± 0.7 | −38.6 ± 0.7 | −72.2 ± 0.3 |
| GII.4-NB-53 | 16 ± 7 | −44.1 ± 1 | −50 ± 5 | −0.7 ± 2 |
| GII.4-NB-56 | 3.8 ± 4 | 10.5 ± 2 | −50.1 ± 5 | −60.6 ± 3 |
| GII.4-NB-76 | 56.7 ± 7 | −50.7 ± 0.4 | −41.4 ± 0.3 | 9.3 ± 0.6 |
| GII.4-NB-82 | 30.1 ± 24 | −16.3 ± 6 | −43.9 ± 2 | −27.7 ± 7 |
| GII.17-NB-2 | 7.5 ± 1 | −72.1 ± 4 | −46.4 ± 5 | 25.7 ± 4 |
| GII.17-NB-7 | 40.9 ± 6 | −85.0 ± 3 | −42.3 ± 0.3 | 42.8 ± 3 |
| GII.17-NB-34 | 4.8 ± 4 | −69.4 ± 4 | −48.1 ± 2 | 21.2 ± 3 |
| GII.17-NB-45 | 101 ± 82 | −22.7 ± 3 | −41.1 ± 3 | −18.4 ± 6 |

TABLE 2
Data collection and refinement statistics for GII.4 Nanobodies

| Complex | GII.4-NB-30 | GII.4-NB-53 |
|---|---|---|
| PBD ID | 8EN1 | 8EN4 |
| Data collection | | |
| Space group | I 1 2 1 | I 2 2 2 |
| Cell dimensions | | |
| a, b, c (Å) | 117.77 62.44 121.40 | 108.41 137.29 140.18 |
| α, β, γ (°) | 90.00 101.24 90.00 | 90 90 90 |
| Resolution range (Å) | 57.75-2.40 (2.49-2.40) | 54.10-2.30 (2.38-2.30) |
| R_{merge}^{a} | 0.153 (0.481) | 0.063 (0.386) |
| I/σI | 5.5 (2.10) | 11.8 (3.1) |
| CC_{1/2} | 0.977 (0.748) | 0.998 (0.931) |
| Completeness (%) | 99.8 (98.7) | 99.7 (98.3) |
| Redundancy | 5.0 (4.6) | 5.1 (5.0) |
| Refinement | | |
| Resolution range (Å) | 46.2-2.4 (2.486-2.4) | 42.91-2.3 (2.382-2.3) |
| No. unique reflections | 34068 (3401) | 46644 (4616) |
| R_{work}^{b}/R_{free}^{c} | 0.2066/0.2635 (0.2788/0.3437) | 0.2145/0.2479 (0.3339/0.3830) |
| No. atoms | 7163 | 6661 |
| Protein | 6890 | 6536 |
| Water | 273 | 113 |
| Ligand | 0 | 12 |
| Average B factors (Å²) | 28.33 | 50.19 |
| Protein | 28.37 | 50.36 |
| Water | 27.28 | 41.01 |
| Ligand | 0 | 45.24 |
| RMSD bond length (Å) | 0.004 | 0.003 |
| RMSD bond angle (°) | 0.61 | 0.53 |
| Ramachandran Plot Statistics^{d} | | |
| Residues | 877 | 841 |
| Most Favored region | 96.78 | 97.84 |
| Allowed Region | 3.11 | 2.04 |
| Outliers | 0.12 | 0.12 |
| Clashscore | 4.02 | 3.81 |

| GII.4-NB-56 | GII.4-NB-76 | GII.4-NB-82 |
|---|---|---|
| 8EN5 | 8EN6 | 8EMY |
| P12₁1 | P2₁2₁2 | P2₁2₁2₁ |
| 61.24 235.38 70.57 | 74.41 119.71 127.82 | 121.02 142.46 207.97 |
| 90.00 104.30 90.00 | 90.00 90.00 90.00 | 90.00 90.00 90.00 |
| 235.38-1.60 (1.63-1.60) | 87.37-1.53 (1.56-1.53) | 117.53-1.7 (1.73-1.70) |
| 0.045 (0.345) | 0.073 (0.661) | 0.035 (0.373) |
| 11.7 (2.5) | 9.3 (1.5) | 19.1 (2.9) |
| 0.997 (0.832) | 0.996 (0.639) | 0.991 (0.712) |
| 98.7 (96.9) | 99.8 (98.4) | 96.9 (77.0) |
| 3.4 (3.5) | 4.4 (4.0) | 4.3 (3.7) |
| 58.85-1.6 (1.657-1.6) | 64.31-1.6 (1.657-1.6) | 61.39-1.7 (1.761-1.7) |
| 249862 (24766) | 150252 (14929) | 379642 (31716) |
| 0.1859/0.2194 (0.2676/0.3054) | 0.1730/0.1944 (0.2237/0.2559) | 0.1772/0.1999 (0.2366/0.2716) |
| 15038 | 7608 | 22388 |
| 13565 | 6666 | 19931 |
| 1317 | 872 | 2317 |
| 192 | 100 | 242 |
| 28.84 | 17.64 | 26.3 |
| 28.25 | 16.57 | 25.33 |
| 34.23 | 25.19 | 34.16 |
| 34.67 | 25.72 | 34.56 |
| 0.014 | 0.015 | 0.005 |
| 1.21 | 1.25 | 0.79 |
| 1736 | 855 | 2563 |
| 97.33 | 98.58 | 97.64 |
| 2.44 | 1.42 | 2.36 |
| 0.23 | 0 | 0 |
| 4.96 | 3.55 | 2.57 |

R_{merge} = [ΣₕΣᵢ|Iₕ − Iₕᵢ|/ΣₕΣᵢIₕᵢ] where Iₕ is the mean of Iₕᵢ observations of reflection h.
^{a}Numbers in parenthesis represent highest resolution shell.
^{b}Rfactor and ^{c}R-free = Σ||Fobs| − |Fcalc|| / Σ|Fobs| × 100 for 95% of recorded data (Rfactor) or 5% data (Rfree)
^{d}Detenmined using MolProbity (10.1002/pro.3330)

TABLE 3
Data collection and refinement statistics for GII.17 Nanobodies

| Complex | GII.17-NB-2 | GII.17-NB-7 |
|---|---|---|
| PDB ID | 8EMZ | 8EN0 |
| Data collection | | |
| Space group | C121 | I222 |
| Cell dimensions | | |
| a, b, c (Å) | 92.96 90.54 59.40 | 74.82 85.46 223.85 |
| α, β, γ (°) | 90.000 116.289 90.000 | 90.0 90.0 90.0 |
| Resolution range (Å) | 61.322-1.401 (1.49-1.40) | 111.926-2.989 (3.17-2.99) |
| R_{merge}^{a} | 0.037 (0.94) | 0.286 (1.859) |
| I/σI | 24.39 (2.19) | 9.10 (1.31) |
| CC_{1/2} | 100 (77.1) | 99.4 (56.6) |
| Completeness (%) | 98.5 (97.7) | 95.5 (97.9) |
| Redundancy | 7.05 (6.97) | 13.4 (13.3) |
| Refinement | | |
| Resolution range (Å) | 34.96-1.401 (1.451-1.401) | 56.29-2.99 (3.097-2.99) |
| No. unique reflections | 85354 (8437) | 14271 (1418) |
| R_{work}^{b}/R_{free}^{c} | 0.1953/0.2196 (0.2944/0.3261) | 0.2489/0.3053 (0.3648/0.3164) |
| No. atoms | 3750 | 3318 |
| Protein | 3400 | 3297 |
| Water | 286 | 21 |
| Ligand | 136 | 0 |
| Average B factors (Å²) | 28.16 | 67.56 |
| Protein | 27.58 | 67.57 |
| Water | 32.99 | 65.15 |
| Ligand | 37.14 | 0 |
| RMSD bond length (Å) | 0.006 | 0.006 |
| RMSD bond angle (°) | 0.84 | 0.89 |
| Ramachandran Plot Statistics^{d} | | |
| Residues | 433 | 425 |
| Most Favored region | 96.97 | 95.25 |
| Allowed Region | 3.03 | 4.04 |
| Outliers | 0 | 0.71 |
| Clashscore | 7.22 | 7.9 |

| GII.10-NB-34 | GII.17-NB-45 |
|---|---|
| 8EN2 | 8EN3 |
| P12₁1 | P12₁1 |
| 66.78 80.53 84.21 | 79.50 71.31 82.62 |
| 90.000 90.025 90.000 | 90.000 113.320 90.000 |
| 80.528-1.850 (1.96-1.85) | 49.082-2.100 (2.23-2.10) |
| 0.074 (1.107) | 0.329 (1.046) |
| 16.48 (1.73) | 5.21 (1.54) |
| 99.9 (70.7) | 97.8 (62.7) |
| 99.1 (98.5) | 99.8 (99.7) |
| 7.10 (7.01) | 6.87 (6.37) |
| 43.88-1.85 (1.917-1.85) | 44.53-2.1 (2.175-2.1) |
| 75469 (7441) | 49637 (4896) |
| 0.2084/0.2344 (0.3323/0.3744) | 0.2126/0.2625 (0.2492/0.3098) |
| 7056 | 7166 |
| 6758 | 6739 |
| 294 | 419 |
| 10 | 8 |
| 38.4 | 21.06 |
| 38.62 | 21.01 |
| 33.68 | 21.65 |
| 20 | 29.38 |
| 0.01 | 0.008 |
| 0.98 | 0.89 |
| 876 | 861 |
| 96.88 | 96.13 |
| 2.78 | 3.87 |
| 0.35 | 0 |
| 3.83 | 4.33 |

R_{merge} = [ΣₕΣᵢ|Iₕ − Iₕᵢ|/ΣₕΣᵢIₕᵢ] where Iₕ is the mean of Iₕᵢ observations of reflection h.
^{a}Numbers in parenthesis represent highest resolution shell.
^{b}Rfactor and ^{c}R-free = Σ||Fobs| − |Fcalc|| / Σ|Fobs| × 100 for 95% of recorded data (Rfactor) or 5% data (Rfree)
^{d}Detenmined using MolProbity (10.1002/pro.3330)

### REFERENCES

1. Kapikian A Z, Wyatt R G, Dolin R, Thornhill T S, Kalica A R, Chanock R M. 1972. Visualization by immune electron microscopy of a 27-nm particle associated with acute infectious nonbacterial gastroenteritis. J Virol 10:1075-81
2. Hall A J, Glass R I, Parashar U D. 2016. New insights into the global burden of noroviruses and opportunities for prevention. Expert Rev Vaccines 15:949-51.10.1080/14760584.2016.1178069
3. Chhabra P, Graaf M, Parra G I, Chan M C, Green K, Martella V, Wang Q, White P A, Katayama K, Vennema H, Koopmans M P G, Vinjé J. 2020. Corrigendum: Updated classification of norovirus genogroups and genotypes. J Gen Virol 101:893.10.1099/jgv.0.001475
4. Chhabra P, de Graaf M, Parra G I, Chan M C, Green K, Martella V, Wang Q, White P A, Katayama K, Vennema H, Koopmans MPG, Vinjé J. 2019. Updated classification of norovirus genogroups and genotypes. J Gen Virol 100:1393-1406.10.1099/jgv.0.001318
5. Prasad B V, Hardy M E, Dokland T, Bella J, Rossmann M G, Estes M K. 1999. X-ray crystallographic structure of the Norwalk virus capsid. Science 286:287-90
6. Harrington P R, Lindesmith L, Yount B, Moe C L, Baric R S. 2002. Binding of Norwalk virus-like particles to ABH histo-blood group antigens is blocked by antisera from infected human volunteers or experimentally vaccinated mice. J Virol 76:12335-43
7. Liu W, Chen Y, Jiang X, Xia M, Yang Y, Tan M, Li X, Rao Z. 2015. A Unique Human Norovirus Lineage with a Distinct HBGA Binding Interface. PLOS Pathog 11: e1005025.10.1371/journal.ppat. 1005025
8. Singh B K, Leuthold M M, Hansman G S. 2015. Human noroviruses' fondness for histo-blood group antigens. J Virol 89:2024-40.10.1128/JVI.02968-14
9. Hansman G S, Biertumpfel C, Georgiev I, Mclellan J S, Chen L, Zhou T, Katayama K, Kwong P D. 2011. Crystal structures of GII.10 and GII.12 norovirus protruding domains in complex with histo-blood group antigens reveal details for a potential site of vulnerability. J Virol 85:6687-701.10.1128/JVI.00246-11
10. Murakami K, Tenge V R, Karandikar U C, Lin S C, Ramani S, Ettayebi K, Crawford S E, Zeng X L, Neill F H, Ayyar B V, Katayama K, Graham D Y, Bieberich E, Atmar R L, Estes M K. 2020. Bile acids and ceramide overcome the entry restriction for GII.3 human norovirus replication in human intestinal enteroids. Proc Natl Acad Sci USA 117:1700-1710.10.1073/pnas.1910138117
11. Estes M K, Ettayebi K, Tenge V R, Murakami K, Karandikar U, Lin S C, Ayyar B V, Cortes-Penfield N W, Haga K, Neill F H, Opekun A R, Broughman J R, Zeng X L, Blutt S E, Crawford S E, Ramani S, Graham D Y, Atmar R L. 2019. Human Norovirus Cultivation in Nontransformed Stem Cell-Derived Human Intestinal Enteroid Cultures: Success and Challenges. Viruses 11.10.3390/v11070638
12. Ettayebi K, Crawford S E, Murakami K, Broughman J R, Karandikar U, Tenge V R, Neill F H, Blutt S E, Zeng X L, Qu L, Kou B, Opekun A R, Burrin D, Graham D Y, Ramani S, Atmar R L, Estes M K. 2016. Replication of human noroviruses in stem cell-derived human enteroids. Science 353:1387-1393.10.1126/science.aaf5211
13. Kilic T, Koromyslova A, Hansman G S. 2019. Structural Basis for Human Norovirus Capsid Binding to Bile Acids. J Virol 93.10.1128/JVI.01581-18
14. Singh B K, Leuthold M M, Hansman G S. 2016. Structural Constraints on Human Norovirus Binding to Histo-Blood Group Antigens. mSphere 1: e00049-16.10.1128/mSphere.00049-16
15. Zhang M, Fu M, Hu Q. 2021. Advances in Human Norovirus Vaccine Research. Vaccines (Basel) 9.10.3390/vaccines9070732
16. Mattison C P, Cardemil C V, Hall A J. 2018. Progress on norovirus vaccine research: public health considerations and future directions. Expert Rev Vaccines doi: 10.1080/14760584.2018.1510327:1-12.10.1080/14760584.2018.1510327
17. Netzler N E, Enosi Tuipulotu D, White P A. 2019. Norovirus antivirals: Where are we now? Med Res Rev 39:860-886.10.1002/med.21545
18. van Loben Sels J M, Green K Y. 2019. The Antigenic Topology of Norovirus as Defined by B and T Cell Epitope Mapping: Implications for Universal Vaccines and Therapeutics. Viruses 11.10.3390/v11050432
19. Lindesmith L C, McDaniel J R, Changela A, Verardi R, Kerr S A, Costantini V, Brewer-Jensen P D, Mallory M L, Voss W N, Boutz D R, Blazeck J J, Ippolito G C, Vinje J, Kwong P D, Georgiou G, Baric R S. 2019. Sera Antibody Repertoire Analyses Reveal Mechanisms of Broad and Pandemic Strain Neutralizing Responses after Human Norovirus Vaccination. Immunity 50:1530-1541.e8.10.1016/j.immuni.2019.05.007
20. Horm K M, D'Souza D H. 2011. Survival of human norovirus surrogates in milk, orange, and pomegranate juice, and juice blends at refrigeration (4 degrees C.). Food Microbiol 28:1054-61.10.1016/j.fm.2011.02.012
21. Su X, Sangster M Y, D'Souza D H. 2010. In Vitro Effects of Pomegranate Juice and Pomegranate Polyphenols on Foodborne Viral Surrogates. Foodborne Pathog Dis doi: 10.1089/fpd.2010.0583.10.1089/fpd.2010.0583
22. Su X, Howell A B, D'Souza D H. 2010. Antiviral effects of cranberry juice and cranberry proanthocyanidins on foodborne viral surrogates—a time dependence study in vitro. Food Microbiol 27:985-91.10.1016/j.fm.2010.05.027
23. Su X, Howell A B, D'Souza D H. 2010. The effect of cranberry juice and cranberry proanthocyanidins on the infectivity of human enteric viral surrogates. Food Microbiol 27:535-40.10.1016/j.fm.2010.01.001
24. Whitehead K, McCue K A. 2010. Virucidal efficacy of disinfectant actives against feline calicivirus, a surrogate for norovirus, in a short contact time. Am J Infect Control 38:26-30.10.1016/j.ajic.2009.03.015
25. Koromyslova A, Tripathi S, Morozov V, Schroten H, Hansman G S. 2017. Human norovirus inhibition by a human milk oligosaccharide. Virology 508:81-89.10.1016/j.virol.2017.04.032
26. Weichert S, Koromyslova A, Singh B K, Hansman S, Jennewein S, Schroten H, Hansman G S. 2016. Structural Basis for Norovirus Inhibition by Human Milk Oligosaccharides. J Virol 90:4843-8.10.1128/JVI.03223-15
27. Koromyslova A D, White P A, Hansman G S. 2015. Treatment of norovirus particles with citrate. Virology 485:199-204.10.1016/j.virol.2015.07.009
28. Hansman G S, Shahzad-Ul-Hussan S, McLellan J S, Chuang G Y, Georgiev I, Shimoike T, Katayama K, Bewley C A, Kwong P D. 2012. Structural basis for norovirus inhibition and fucose mimicry by citrate. J Virol 86:284-92.10.1128/JVI.05909-11
29. Ruoff K, Devant J M, Hansman G. 2022. Natural extracts, honey, and propolis as human norovirus inhibitors. Sci Rep 12:8116.10.1038/s41598-022-11643-5
30. Jovcevska I, Muyldermans S. 2020. The Therapeutic Potential of Nanobodies. BioDrugs 34:11-26.10.1007/s40259-019-00392-z
31. Li T, Cai H, Yao H, Zhou B, Zhang N, van Vlissingen M F, Kuiken T, Han W, GeurtsvanKessel C H, Gong Y, Zhao Y, Shen Q, Qin W, Tian X X, Peng C, Lai Y, Wang Y, Hutter C A J, Kuo S M, Bao J, Liu C, Wang Y, Richard A S, Raoul H, Lan J, Seeger M A, Cong Y, Rockx B, Wong G, Bi Y, Lavillette D, Li D. 2021. A synthetic nanobody targeting RBD protects hamsters from SARS-COV-2 infection. Nat Commun 12:4635.10.1038/s41467-021-24905-z
32. Detalle L, Stohr T, Palomo C, Piedra P A, Gilbert B E, Mas V, Millar A, Power U F, Stortelers C, Allosery K, Melero J A, Depla E. 2016. Generation and Characterization of ALX-0171, a Potent Novel Therapeutic Nanobody for the Treatment of Respiratory Syncytial Virus Infection. Antimicrob Agents Chemother 60:6-13.10.1128/AAC.01802-15
33. Ibanez L I, De Filette M, Hultberg A, Verrips T, Temperton N, Weiss R A, Vandevelde W, Schepens B, Vanlandschoot P, Saelens X. 2011. Nanobodies with in vitro neutralizing activity protect mice against H5N1 influenza virus infection. J Infect Dis 203:1063-72.10.1093/infdis/jiq168
34. Koromyslova A D, Hansman G S. 2017. Nanobodies targeting norovirus capsid reveal functional epitopes and potential mechanisms of neutralization. PLOS Pathog 13: e1006636.10.1371/journal.ppat.1006636
35. Koromyslova A D, Hansman G S. 2015. Nanobody binding to a conserved epitope promotes norovirus particle disassembly. J Virol 89:2718-30.10.1128/JVI.03176-14
36. Parra G I, Bok K, Taylor R, Haynes J R, Sosnovtsev S V, Richardson C, Green K Y. 2012. Immunogenicity and specificity of norovirus Consensus GII.4 virus-like particles in monovalent and bivalent vaccine formulations. Vaccine 30:3580-6.10.1016/j.vaccine.2012.03.050
37. Hansman G S, Doan L T, Kguyen T A, Okitsu S, Katayama K, Ogawa S, Natori K, Takeda N, Kato Y, Nishio O, Noda M, Ushijima H. 2004. Detection of norovirus and sapovirus infection among children with gastroenteritis in Ho Chi Minh City, Vietnam. Arch Virol 149:1673-88
38. Koromyslova A D, Morozov V A, Hefele L, Hansman G S. 2019. Human Norovirus Neutralized by a Monoclonal Antibody Targeting the Histo-Blood Group Antigen Pocket. J Virol 93.10.1128/jvi.02174-18
39. Kabsch W. 1993. Automatic processing of rotation diffraction data from crystals of initially unknown symmetry and cell constants. J Appl Cryst 26:795-800
40. McCoy A J, Grosse-Kunstleve R W, Adams P D, Winn M D, Storoni L C, Read R J. 2007. Phaser crystallographic software. Journal of Applied Crystallography 40:658-674
41. Emsley P, Lohkamp B, Scott W G, Cowtan K. 2010. Features and development of Coot.

### Acta Crystallogr D Biol Crystallogr 66:486-501

42. Adams P D, Afonine P V, Bunkóczi G, Chen V B, Davis I W, Echols N, Headd J J, Hung L-W, Kapral G J, Grosse-Kunstleve R W, McCoy A J, Moriarty N W, Oeffner R, Read R J, Richardson D C, Richardson J S, Terwilliger T C, Zwart P H. 2010. PHENIX: a comprehensive Python-based system for macromolecular structure solution. Acta Crystallogr D Biol Crystallogr 66:213-221
43. Morris A L, Mac Arthur M W, Hutchinson E G, Thornton J M. 1992. Stereochemical quality of protein structure coordinates. Proteins 12:345-64.10.1002/prot.340120407
44. Chen V B, Arendall W B, 3rd, Headd J J, Keedy D A, Immormino R M, Kapral G J, Murray L W, Richardson J S, Richardson D C. 2010. MolProbity: all-atom structure validation for macromolecular crystallography. Acta Crystallogr 66:12-Crystallogr D Biol 21.10.1107/S0907444909042073
45. Krissinel E, Henrick K. 2007. Inference of macromolecular assemblies from crystalline state. J Mol Biol 372:774-97.10.1016/j.jmb.2007.05.022
46. Koromyslova A D, Devant J M, Kilic T, Sabin C D, Malak V, Hansman G S. 2020. Nanobody-Mediated Neutralization Reveals an Achilles Heel for Norovirus. J Virol 94.10.1128/jvi.00660-20
47. Ruoff K, Kilic T, Devant J, Koromyslova A, Ringel A, Hempelmann A, Geiss C, Graf J, Haas M, Roggenbach I, Hansman G. 2019. Structural Basis of Nanobodies Targeting the Prototype Norovirus. J Virol 93: e02005-18.10.1128/JVI.02005-18
48. Hansman G S, Natori K, Shirato-Horikoshi H, Ogawa S, Oka T, Katayama K, Tanaka T, Miyoshi T, Sakae K, Kobayashi S, Shinohara M, Uchida K, Sakurai N, Shinozaki K, Okada M, Seto Y, Kamata K, Nagata N, Tanaka K, Miyamura T, Takeda N. 2006. Genetic and antigenic diversity among noroviruses. J Gen Virol 87:909-19.10.1099/vir.0.81532-0
49. Lindesmith L C, Mallory M L, Debbink K, Donaldson E F, Brewer-Jensen P D, Swann E W, Sheahan T P, Graham R L, Beltramello M, Corti D, Lanzavecchia A, Baric R S. 2018. Conformational Occlusion of Blockade Antibody Epitopes, a Novel Mechanism of GII.4 Human Norovirus Immune Evasion. mSphere 3.10.1128/mSphere.00518-17
50. Lindesmith L C, Ferris M T, Mullan C W, Ferreira J, Debbink K, Swanstrom J, Richardson C, Goodwin R R, Baehner F, Mendelman P M, Bargatze R F, Baric R S. 2015. Broad blockade antibody responses in human volunteers after immunization with a multivalent norovirus VLP candidate vaccine: immunological analyses from a phase I clinical trial. PLOS Med 12: e1001807.10.1371/journal.pmed.1001807
51. Doerflinger S Y, Tabatabai J, Schnitzler P, Farah C, Rameil S, Sander P, Koromyslova A, Hansman G S. 2016. Development of a Nanobody-Based Lateral Flow Immunoassay for Detection of Human Norovirus. mSphere doi: 10.1128/mSphere.00219-16: e00219-16.10.1128/mSphere.00219-16
52. Singh B K, Koromyslova A, Hefele L, Gurth C, Hansman G S. 2015. Structural evolution of the emerging 2014/15 GII.17 noroviruses. J Virol 90:2710-5.10.1128/JVI.03119-15
53. Singh B K, Koromyslova A, Hefele L, Gürth C, Hansman G S. 2015. Structural Evolution of the Emerging 2014-2015 GII.17 Noroviruses. J Virol 90:2710-5.10.1128/jvi.03119-15
54. Dai Y C, Xia M, Huang Q, Tan M, Qin L, Zhuang Y L, Long Y, Li J D, Jiang X, Zhang X F. 2017. Characterization of Antigenic Relatedness between GII.4 and GII.17 Noroviruses by Use of Serum Samples from Norovirus-Infected Patients. J Clin Microbiol 55:3366-3373.10.1128/jcm.00865-17
55. Zhang X F, Huang Q, Long Y, Jiang X, Zhang T, Tan M, Zhang Q L, Huang Z Y, Li Y H, Ding Y Q, Hu G F, Tang S, Dai Y C. 2015. An outbreak caused by GII.17 norovirus with a wide spectrum of HBGA-associated susceptibility. Sci Rep 5:17687.10.1038/srep17687
56. Zhang X F, Tan M, Chhabra M, Dai Y C, Meller J, Jiang X. 2013. Inhibition of histo-blood group antigen binding as a novel strategy to block norovirus infections. PLOS One 8: e69379.10.1371/journal.pone.0069379
57. Chen Y, Tan M, Xia M, Hao N, Zhang X C, Huang P, Jiang X, Li X, Rao Z. 2011. Crystallography of a Lewis-binding norovirus, elucidation of strain-specificity to the polymorphic human histo-blood group antigens. PLOS Pathog 7: e1002152.10.1371/journal.ppat.1002152
58. Koromyslova A D, Morozov V A, Hefele L, Hansman G S. 2018. Human Norovirus Neutralized by a Monoclonal Antibody Targeting the HBGA Pocket. J Virol doi: 10.1128/JVI.02174-18.10.1128/JVI.02174-18
59. Doerflinger S Y, Weichert S, Koromyslova A, Chan M, Schwerk C, Adam R, Jennewein S, Hansman G S, Schroten H. 2017. Human Norovirus Evolution in a Chronically Infected Host. mSphere 2.10.1128/mSphere.00352-16
60. Debbink K, Lindesmith L C, Donaldson E F, Costantini V, Beltramello M, Corti D, Swanstrom J, Lanzavecchia A, Vinje J, Baric R S. 2013. Emergence of New Pandemic GII.4 Sydney Norovirus Strain Correlates With Escape From Herd Immunity. J Infect Dis doi: 10.1093/infdis/jit370.10.1093/infdis/jit370
61. Lindesmith L C, Debbink K, Swanstrom J, Vinje J, Costantini V, Baric R S, Donaldson E F. 2012. Monoclonal antibody-based antigenic mapping of norovirus GII.4-2002. J Virol 86:873-83.10.1128/JVI.06200-11
62. Lindesmith L C, Donaldson E F, Lobue A D, Cannon J L, Zheng D P, Vinje J, Baric R S. 2008. Mechanisms of GII.4 norovirus persistence in human populations. PLOS Med 5: e31.10.1371/journal.pmed.0050031
63. Rockx B H, Vennema H, Hoebe C J, Duizer E, Koopmans M P. 2005. Association of histo-blood group antigens and susceptibility to norovirus infections. J Infect Dis 191:749-54.10.1086/427779
64. Tan M, Hegde R S, Jiang X. 2004. The P domain of norovirus capsid protein forms dimer and binds to histo-blood group antigen receptors. J Virol 78:6233-42.10.1128/JVI.78.12.6233-6242.2004
65. Harrington P R, Vinje J, Moe C L, Baric R S. 2004. Norovirus capture with histo-blood group antigens reveals novel virus-ligand interactions. J Virol 78:3035-45
66. Tan M, Huang P, Meller J, Zhong W, Farkas T, Jiang X. 2003. Mutations within the P2 domain of norovirus capsid affect binding to human histo-blood group antigens: evidence for a binding pocket. J Virol 77:12562-71
67. Lindesmith L, Moe C, Marionneau S, Ruvoen N, Jiang X, Lindblad L, Stewart P, LePendu J, Baric R. 2003. Human susceptibility and resistance to Norwalk virus infection. Nat Med 9:548-53.10.1038/nm860
68. Huang P, Farkas T, Marionneau S, Zhong W, Ruvoen-Clouet N, Morrow A L, Altaye M, Pickering L K, Newburg D S, LePendu J, Jiang X. 2003. Noroviruses bind to human ABO, Lewis, and secretor histo-blood group antigens: identification of 4 distinct strain-specific patterns. J Infect Dis 188:19-31.10.1086/375742
69. Alvarado G, Salmen W, Ettayebi K, Hu L, Sankaran B, Estes M K, Venkataram Prasad B V, Crowe J E, Jr. 2021. Broadly cross-reactive human antibodies that inhibit genogroup I and II noroviruses. Nat Commun 12:4320.10.1038/s41467-021-24649-w
70. Hansman G S, Taylor D W, Mclellan J S, Smith T J, Georgiev I, Tame J R, Park S Y, Yamazaki M, Gondaira F, Miki M, Katayama K, Murata K, Kwong P D. 2012. Structural basis for broad detection of genogroup II noroviruses by a monoclonal antibody that binds to a site occluded in the viral particle. J Virol 86:3635-46.10.1128/JVI.06868-11
71. Schroten H, Hanisch F G, Hansman G S. 2016. Human norovirus interactions with histo-blood group antigens and human milk oligosaccharides. J Virol doi: 10.1128/JVI.00317-16.10.1128/JVI.00317-16
72. Alvarado G, Ettayebi K, Atmar R L, Bombardi R G, Kose N, Estes M K, Crowe J E, Jr. 2018. Human Monoclonal Antibodies That Neutralize Pandemic GII.4 Noroviruses. Gastroenterology 155:1898-1907.10.1053/j.gastro.2018.08.039

1. A single domain antibody that is directed against a P domain of a norovirus, wherein the single domain antibody binds to or interacts with one or more residues of a P2 subdomain thereof.

2. The single domain antibody of claim 1, wherein the P2 subdomain comprises, consists of or consists essentially of residues 275 to 417 or residues 276 to 418 of a VP1 protein of the norovirus.

3. The single domain antibody of claim 1, which binds to or interacts with one or more residues of the P domain selected from the group consisting of LYS-329, THR-344, SER-355, ALA-356, ASP-357, GLU-368, ASP-391, THR-394, ASN-398, GLN-401 and GLY-443 according to the amino acid numbering of a VP1 protein of a GII.4 genotype.

4. The single domain antibody of claim 1, which binds to or interacts with one or more residues of the P domain selected from the group consisting of ASN-295, GLN-296, ARG-297, ARG-299, GLN-352, TRP-354, GLN-361, ARG-372, SER-374, ASN-392, ASP-393, ASP-394, ASP-395, ASP-396, SER-441, GLY-442, GLY-443 and TYR-444 according to the amino acid numbering of a VP1 protein of a GII.17 genotype.

5. The single domain antibody of claim 1, comprising:
(a) a CDR1 that comprises an amino acid sequence of ASGRFFSSYA (SEQ ID NO: 5), ASGRTFSSY (SEQ ID NO: 8), RTDSEST (SEQ ID NO: 11), SGTIFSIDA (SEQ ID NO: 14), or a variant thereof;
(b) a CDR2 that comprises an amino acid sequence of ISWSGGST (SEQ ID NO: 6, TGSGD (SEQ ID NO: 9), WRYA (SEQ ID NO: 12), QAPGKQRE (SEQ ID NO: 15), or a variant thereof; and
(c) a CDR3 that comprises an amino acid sequence of AREGAYYPDSYYRTVRYD (SEQ ID NO: 7), YRTGGPPQW (SEQ ID NO: 10), RYIYGSLSDSGSYDN (SEQ ID NO: 13), AKPPTYYSLEPWGKGT (SEQ ID NO: 16), or a variant thereof.

6. The single domain antibody of claim 5, comprising:
(i) the CDR1 that comprises the amino acid sequence of ASGRFFSSYA (SEQ ID NO: 5) or a variant thereof, the CDR2 that comprises the amino acid sequence of ISWSGGST (SEQ ID NO: 6) or a variant thereof, and the CDR3 that comprises the amino acid sequence of AREGAYYPDSYYRTVRYD (SEQ ID NO: 7) or a variant thereof;
(ii) the CDR1 that comprises the amino acid sequence of ASGRTFSSY (SEQ ID NO: 8) or a variant thereof, the CDR2 that comprises the amino acid sequence of TGSGD (SEQ ID NO: 9) or a variant thereof, and the CDR3 that comprises the amino acid sequence of YRTGGPPQW (SEQ ID NO: 10) or a variant thereof;
(iii) the CDR1 that comprises the amino acid sequence of RTDSEST (SEQ ID NO: 11) or a variant thereof, the CDR2 that comprises the amino acid sequence of WRYA (SEQ ID NO: 12) or a variant thereof, and the CDR3 that comprises the amino acid sequence of RYIYGSLSDSGSYDN (SEQ ID NO: 13) or a variant thereof; or
(iv) the CDR1 that comprises the amino acid sequence of SGTIFSIDA (SEQ ID NO: 14) or a variant thereof, the CDR2 that comprises the amino acid sequence of QAPGKQRE (SEQ ID NO: 15) or a variant thereof, and the CDR3 that comprises the amino acid sequence of AKPPTYYSLEPWGKGT (SEQ ID NO: 16) or a variant thereof.

7. The single domain antibody of claim 5, comprising, consisting of or consisting essentially of an amino acid sequence selected from SEQ ID NOs: 1 to 4, or a fragment, variant or derivative thereof.

8. The single domain antibody of claim 1, wherein the norovirus is of a GII genogroup.

9.-10. (canceled)

11. The single domain antibody of claim 1, wherein the single domain antibody has been at least partly humanized.

12. An antigen binding molecule comprising the single domain antibody of claim 1.

13. The antigen binding molecule of claim 12, wherein the antigen binding molecule is or comprises a monovalent single domain antibody, a multivalent single domain antibody, or a multispecific single domain antibody.

14. The antigen binding molecule of claim 13, wherein the antigen binding molecule is a multispecific single domain antibody comprising a further single domain antibody that is directed to a P1 subdomain of a norovirus.

15. The antigen binding molecule of claim 12, wherein the antigen binding molecule is or comprises an immunoconjugate.

16. The antigen binding molecule of claim 15, wherein the immunoconjugate comprises one or more of a detectable marker, a therapeutic agent, a half-life extender and a nanocarrier.

17. An isolated nucleic acid comprising a nucleotide sequence which encodes, or is complementary to a nucleotide sequence which encodes, the single domain antibody of claim 1.

18. The isolated nucleic acid of claim 17, wherein the isolated nucleic acid is or comprises mRNA.

19.-23. (canceled)

24. A method of diagnosing or monitoring a norovirus infection and/or a disease, disorder or condition associated therewith in a subject, said method including the step of contacting the subject and/or a biological sample from the subject with the single domain antibody of claim 1.

25. The method of claim 24, which further includes the step of detecting and/or measuring a level of antigen binding to the single domain antibody or the antigen binding molecule.

26. (canceled)

27. A method of treating or preventing a norovirus infection and/or a disease, disorder or condition associated therewith in a subject including the step of administering a therapeutically effective amount of the single domain antibody of claim 1 to thereby treat or prevent the norovirus infection and/or disease, disorder or condition associated therewith in the subject.

28.-29. (canceled)

30. A method for inactivating or neutralising a norovirus associated with a substrate or surface, said method including the step of contacting the substrate with an effective amount of the single domain antibody of claim 1 to thereby inactivate or neutralise the norovirus associated with the substrate or surface.
